(19) United States
(12) Patent Application Publication
(10) Pub. No.: US 2025/0232849 A1
(43) Pub. Date: Jul. 17, 2025

(54) SYSTEM AND METHOD OF MANIFOLD KERNELIZATION OF MOLECULAR SURFACE

(71) Applicant: Purdue Research Foundation, West Lafayette, IN (US)

(72) Inventor: Tonglei Li, West Lafayette, IN (US)

(73) Assignee: Purdue Research Foundation, West Lafayette, IN (US)

(21) Appl. No.: 19/170,757

(22) Filed: Apr. 4, 2025

Related U.S. Application Data
(63) Continuation-in-part of application No. PCT/US2024/025941, filed on Apr. 24, 2024.
(60) Provisional application No. 63/546,710, filed on Oct. 31, 2023.

(51) Int. Cl. G16C 20/70; G16C 20/20

(52) U.S. Cl. CPC G16C 20/70; G16C 20/20

(57) ABSTRACT

Methods, systems, devices and/or apparatuses for encoding electronic quantities from a molecular surface to a molecular representation through manifold kernelization for use as inputs in deep learning applications for the prediction of molecular properties.

### CROSS-REFERENCE TO RELATED APPLICATIONS

The present application is a continuation in part of International Patent Application PCT/US2024/025941, filed Apr. 24, 2024, which claims the benefit of U.S. Provisional Application 63/546,710, filed on Oct. 31, 2023, the disclosures of which are incorporated by reference in their entireties.

### TECHNICAL FIELD

The present disclosure relates to computational methods, systems, and non-transitory computer-readable media for encoding electronic quantities from a molecular surface to a molecular representation for the prediction of molecular properties and de novo design in deep learning applications.

### BACKGROUND

Drug discovery and development is often described as finding a needle in a haystack. In fact, searching for a drug in the chemical space (10\hat{ }63 molecules and more) is much more daunting, especially when dozens or more molecular descriptors are used to define the dimensions of the space. Identifying and optimizing a fitting chemical through the drug discovery and development processes is costly and laborious. As a result, less than 1 out of 10\hat{ }4 molecules brought to the drug research pipeline can make it to the clinic. The extreme complexities in evaluating a lead compound's efficacy, toxicity, bioavailability, and developability have long desired in silico approaches to predict the physical, biological, and pharmacological properties of molecules based on their structures. Recent advances in deep learning and the availability of extensive chemical data provide great opportunities to facilitate drug research; however, prediction of molecular functionalities and de novo drug design have not yet made great strides. A root obstacle is the Curse of Dimensionality (COD) when exploring the chemical space by current schemes of molecular representation.

A molecule is classically depicted by a graph of nodes and lines. Various featurization schemes have been empirically evolved from the graphic convention. When predicting molecular interactions and properties, it is a common practice to use as many descriptors (and fingerprints) as possible to embody the chemistry of a molecule. However, the more features are used, the more exponentially the dimensionality of the chemical space expands. The COD leaves the vast space scarcely covered by available experimental data, drastically deteriorating the predictive power of data-fitted structure-property models.

In silico drug research counts on computable features of molecules to exploit available chemical data and explore the underlying quantitative relationships between molecular features and conceivable physicochemical and therapeutic properties. Thanks to the rapid growth in data collection, cloud storage, and online access, various datacentric approaches have become a staple in modern drug discovery and development. At the same time, prediction by first principles remains impractical, especially when dealing with multifaceted phenomena (e.g., dissolution and binding to a flexible protein). Most featurization schemes are empirically evolved from the conventional depiction of a molecule as a graph of atoms, resulting in many forms of descriptors and fingerprints. A descriptor is a value of a molecule's 1-, 2-, or 3-D feature (e.g., number of hydrogen-bonding donors). A fingerprint consists of an alphanumerical string (e.g., Weininger, D., Smiles, “A Chemical Language and Information-System.1. Introduction to Methodology and Encoding Rules” [Journal of Chemical Information and Computer Sciences 1988, 28, 31-36]) or a digital vector (e.g., Rogers, D.; Hahn, M., “Extended-Connectivity Fingerprints” [Journal of Chemical Information and Modeling 2010, 50, 742-754]) to encode the holistic chemical constitution and bonding information. However, no single descriptor or fingerprint can fully capture the chemistry of a molecule's functionality. It is thus not uncommon to see in a study that dozens or even hundreds of features are used to represent a molecule. The practice, unfortunately, leads to COD. Considering the sheer number of potential molecule candidates (>10\hat{ }63), the Curse is additionally exacerbated.

The chemical space formed by conventional descriptors (and fingerprints) becomes too sparse to cover by chemical data, resulting in model overfitting. The predictive power of any data-derived model exponentially deteriorates as the number of descriptor dimensions increases. The COD has primarily impeded the data-driven drug discovery and development, making it critically desired to create low-dimensional molecular features that accurately capture the chemistry of a molecule.

Earlier work has provided some attempts to improve the computation of the chemical space. See, e.g., Li, T. L.; Liu, S. B.; Feng, S. X.; Aubrey, C. E., “Face-Integrated Fukui Function: Understanding Wettability Anisotropy of Molecular Crystals from Density Functional Theory” (Journal of the American Chemical Society 2005, 127, 1364-1365); Zhang, M. T.; Li, T. L., “Intermolecular Interactions in Organic Crystals: Gaining Insight from Electronic Structure Analysis by Density Functional Theory” (Crystengcomm 2014, 16, 7162-7171); and Bhattacharjee, R.; Verma, K.; Zhang, M.; Li, T. L., “Locality and Strength of Intermolecular Interactions in Organic Crystals: Using Conceptual Density Functional Theory (Cdft) to Characterize a Highly Polymorphic System” (Theoretical Chemistry Accounts 2019, 138).

### SUMMARY

The present disclosure involves, in one embodiment, a method for creating a representation of a molecule as a chemically authentic and dimensionally reduced feature for computing molecular interactions and pertinent properties. A plurality of molecules is measured by observation of electronic patterns on a three-dimensional molecular surface. A manifold kernelization of the observed electronic patterns is created, the kernelization having a two-dimensional representation of the observed electronic patterns. The two-dimensional representation is associated with chemical properties. Such chemical properties may include, e.g., solubility (a combination of hydrophobicity and lattice energy), developability, permeability, physiochemical properties such as absorption, distribution, metabolism, excretion, and/or toxicity (ADMET), and the like. Dimensional reduction through manifold kernelization is performed for at least one of the plurality of molecules. A data structure is created based on the molecular surface and the chemical properties.

In one embodiment, a database of molecular representations includes a plurality of data structures. The data structures include manifold kernelization of the observed electronic patterns on a molecular surface, at least one of the observed electronic patterns being dimensionally reduced via manifold kernelization, with an association of the manifold kernelization with chemical properties.

In yet a further embodiment, a method of determining chemical properties of observed molecule electronic patterns using a database having a plurality of data structures representing molecular surfaces and associated chemical properties is provided. A quantum calculation of the observed molecule electronic patterns is created, along with a manifold kernelization of molecular surface (MKMS) representation of the observed molecule using dimensionality reduction. The MKMS representation of the observed molecule is used as an input with a neural network utilizing the database to identify properties of the observed molecule.

Methods designing molecules for particular bioavailable properties can involve several steps. For example, in a process of manifold embedding of molecular surface, a latent space of molecular projections on a functional surface is created. A feature matrix is then kernelized based on the latent space to an adjacency matrix matching molecular projections with functional properties. According to this method, the three-dimensional surface is cut and stretched over a two-dimensional plane prior to kernelized, which can disrupt the underlying geospatial patterns of the molecule.

By contrast, MKMS of the present disclosure directly kernelizes quantum molecular data without the intermediate step of generating a feature matrix, thus leaving the 3-dimensional structure completely intact. In particular, MKMS utilizes kernel learning to capture directly information of electronic and quantum attributes, which may include, without limitation, electrostatic potential (ESP) and/or Fukui function quantities of a target molecule's iso-electronic density surface. In that regard, MKMS offers (1) information completeness, i.e., no information loss; (2) robustness against dimensionality reduction; and (3) mathematically differentiability, ensuring the continuity of chemical space, which can be important for generative AI prediction of molecular structures.

To that end, in one aspect, a one non-transitory computer-readable medium is disclosed that includes instructions that, when executed by at least one processor, cause the at least one processor to: (1) receive a dataset of electronic quantities across a manifold topology of a molecule of interest; (2) perform manifold regression on the electronic quantities to encode quantum information of the molecule in a covariant matrix (kernel), wherein the covariant matrix captures mutual relationships among the electronic quantities and the manifold topology; and (3) export the covariance matrix as a symmetric semi-positive definite matrix for use as an input of molecular representation to a neural network model configured to utilize the matrix for predicting molecular properties. Electronic quantities according to examples can include ESP and/or Fukui function quantities.

A molecular surface according to embodiments herein can be transformed using, for example, reduced-rank Sparse Gaussian Process (SGP) with Spectral Mixture (SM) as the kernel function. According to examples, the SGP utilizes a set of inducing points chosen as the closest vertices to the respective atoms of a molecule, resulting in a kernel matrix being n\hat{ }2, where n is the number of atoms in the molecule. In the same or other examples, the covariance function of the spectral solution of a manifold can be identified through an eigen decomposition of the Graph Laplacian of a molecular surface. In the same or yet other examples, the hyperparameters used in the reduced-rank SGP, particularly those associated with the means and variances of the Gaussian functions can be set to 1, to improve convergence. The kernel matrix can also be modified via eigen value reduction to a number near 0, generally between 0.001 and 0.00001, to allow for matrix or other value optimization. In one example, a combination of resulting kernel matrices, derived from different molecular surfaces from the same molecule, can be used to predict the molecule's chemical properties.

According to an embodiment, dimensionality reduction may be used to project the data from a Euclidean tangent space or other similar space to a Riemannian manifold or other similar space. Projection can also be used to project the data from a Riemannian manifold or other similar space to a Euclidean tangent or other similar space.

Any neural network(s) (or other machine or deep learning models) configured and/or trained to receive a single positive definite (SPD) matrix or derivates thereof as input to predict an embedded molecule's chemical properties can be used consistent with the present disclosure, including, in one example, a trained SPDNet Attention model. Such chemical properties include, e.g., solubility (a combination of hydrophobicity and lattice energy), developability, permeability, and physiochemical properties such as absorption, distribution, metabolism, excretion, and/or toxicity (ADMET). According to examples, the model of embodiments of the invention involves a Deep Sets-based Graph and Self-Attention Network with input from a computational chemistry approach to evaluating electron density and one or more chemical property values as labels. As intermolecular interactions are very important in determining the solubility of a molecule and are represented well by electron density, electron density of the drug molecules may be used computationally to predict solubility. A good dataset is critical to the success of a solubility prediction algorithm. In one embodiment, the algorithm uses molecules found in the first and second Solubility Challenge conducted by Avdeef et al., a solubility prediction challenge which pitted human predictors against machine learning algorithms. There are a total of 90 molecules with an inherently normal distribution of solubility values.

The method of generating unique descriptors of intermolecular interactions in the precursor MEMS method was accomplished by: 1) using quantum mechanical conceptual density functional theory (CDFT) calculations to generate measures of electron density from crystal structures; 2) generating the molecular surface by determining the contribution of electrons in the crystal structure from individual molecules; and 3) embedding the CDFT calculated values onto the three-dimensional (3D) molecular surface, which is then projected into two dimensions (2D) using a stochastic neighborhood embedding approach. From each atom location in the 2D space, radially and angularly distributed electrostatic potential and Fukui function values are taken as input for the neural network.

The present method of generating unique descriptors of intermolecular interactions can be performed using MKMS. In one example, the MKMS method includes the steps of: (1) receiving a dataset of electronic quantities across a manifold topology of a molecule of interest; (2) performing manifold regression on the electronic quantities to encode quantum information of the molecule in a covariant matrix (kernel), wherein the covariant matrix captures mutual relationships among the electronic quantities and the manifold topology; and (3) exporting the covariance matrix as a symmetric semi-positive definite matrix for use as an input to a neural network model configured to utilize the matrix for predicting molecular properties.

In chemical learning, a molecule is described in a digitalized form to develop quantitative structure-activity or structure-property relationships (QSAR or QSPR) by a machine learning model. A molecule is often represented as an assembly or set of individual descriptors, such as molecular weight, dipole moment, and number of single bonds. Moreover, the conventional depiction of a molecule as a graph of nodes and lines signifying atoms and bonds has initiated various description or fingerprinting schemes, such as SMILES, and ECFP. A descriptor is generally of a 1-, 2-, or 3-D feature of a molecule; the elemental composition and chemical connectivity may also be encoded as a fingerprint or alphanumeric string. While benchmarking studies have been conducted to show one representation outperforms another, in principle, as long as it could fully differentiate molecules (in a molecular dataset), a set of descriptors, a graph representation, or a fingerprint would assume a one-to-one connection or function with a molecular property that may be approximated by machine learning. Nonetheless, there remain two interweaved challenges when applying a molecular description in data-driven chemical learning. The first one stems from the so-called Curse of Dimensionality (COD).

A set of descriptors or a fingerprint bears the dimensionality of its features. As the dimensionality increases, the covering of the chemical space by the same amount of data becomes exponentially reduced. For instance, if each descriptor could have 50% of its dimension covered by data, the coverage in 3-D would be 12.5% and in 10-D, it would become merely 0.1%. Consequently, model over-fitting is likely entailed in a hyperspace, resulting in poor predictability. In a high-dimensional space, the distances between any two points become approximately identical, making any distance-based classification or regression prediction ineffective. Considering the enormous number of potential molecules in chemical space, a machine learning effort with a few dozen, or more, dimensions of molecular features would require far more experimental data than what is available. It is commonly assumed, implicitly or explicitly, that the intended molecules in a study reside in a significantly constrained subspace or on an underlying manifold that is defined by far fewer dimensions. Effectively reducing the dimensionality of molecular descriptors or chemical features is thus necessitated when developing data-driven predictions. On the other hand, multiple steps of dimensionality reduction may demote the eventual discerning power and resolution of molecules and impede machine learning architectures to infer the underlying or true function. The quandary might be well reflective of the observation by Hughes in 1968 that the predicting power of a classification model first increases and then declines as the number of descriptors increases.

The empirical nature of selection and utilization of conventional descriptors presents another challenge in chemical learning. The totality of coalescing multiple descriptors in a study may not fully or accurately capture the chemistry of a molecule. Correlations are common among descriptors, requiring careful examination and removal of those that add little chemical intuition. Selection of suitable descriptors is subjected to trials and errors and at the discretion of one's preference and experience. While a set of molecule-discerning descriptors (or a fingerprint) could potentially lead to fitting of a causal function, the complexity to develop a machine learning model and untangle the one-to-one connection between the input representation and output property is directly influenced by how a molecule is featurized. When a representation carries no direct information of the intended property, multiple latent functions with differing dimensionalities are necessarily involved to bridge the “domain distance” between the input and output, requiring complicated machine learning models (and chemical rules), as well as facing the perspective of the COD and in need of a large amount of high-quality data. It is thus desired to represent a molecule by fundamentally derived quantities that orthogonally preserve the chemical information of molecules and directly connect with molecular properties.

As molecular interactions are best described by quantum mechanics, featurization of electronic structures and attributes may overcome the above-mentioned challenges when learning to predict molecular properties that stem from molecular interactions. There have been many efforts to capture electronic quantities for machine learning. One general approach is to augment a molecular graph with electronic attributes. The adjacency matrix of a molecule may be weighted by electronic or chemical properties localized to atoms or atomic pairs. One such development is Coulomb matrix; electron density-weighted connectivity matrix (EDWCM) is another concept, in which the electron density at the bond critical point (BCP) is recorded for each bonded pair. With a similar footing of partitioning the electron density, electron localization-delocalization matrix (LDM) is devised with localized electron values assigned to the diagonal elements (atoms) and de-localized values assigned to the off-diagonal pairs. There are also efforts to integrate electronic quantities that are derived from the second-order perturbation analysis (SOPA) in the context of natural bond orbital (NBO) theory in molecular graph for machine learning. In the recent development of OrbNet-Equi, the molecular representation may be regarded as adjacency matrix where each element is a concatenated vector of respective parameters of single-electron operators, such as Fock and density matrices, on atomic orbitals. Because of the underpinning of molecular topology, graph neural networks (GNNs), including convolutional GNNs (CGNNs) and message passing NNs (MPNNs), are often utilized to handle these representations. Alternatively, there are approaches to discretize the space of a molecule by a finite grid to retain the electron density and pertinent attributes for machine learning. Two noteworthy efforts are PIXEL, where the valence-only electron densities are partitioned to voxels, and CoMFA, where interaction energies against a probe atom traversing at the pre-determined grid points are recorded. These representations are however not invariant to rotation or orientation of a molecule, potentially limiting their usage.

Given the premise of representing molecules for chemical learning, a new concept of lower-dimensional embeddings of electron densities and local electronic attributes on a molecular surface is reported. The concept of manifold embedding of molecular surface (MEMS) is aimed to preserve the quantum chemical information of molecular interactions by translation- and rotation-invariant feature vectors. The conceptualization of the precursor MEMS was rooted in studies of intermolecular interactions. The hard and soft acids and bases principle (HSAB) was exploited within the framework of conceptual density functional theory (CDFT) to characterize intermolecular interactions in organic crystals. These studies unveiled that Fukui functions, electrostatic potential (ESP), and other density functional-derived quantities at the interface between two molecules quantitatively determine the locality and strength of intermolecular interactions. A crucial finding was that the electronic properties of the single molecule—other than those of the explicitly interacted molecule pair-bear the information of both the strength and locality of intermolecular interactions. This provided the motivation explore the intrinsic electronic attributes of a single molecule to study intermolecular interactions, more recently by deep learning.

Treating a molecular surface as a manifold, the precursor MEMS strategy aligned with manifold learning—a manifold assumes a lower-dimensional embedding, which may be uncovered by computation. A molecular surface is not a physical quantity but a chemical perception to partition the electron density of a molecule. It marks the boundary where intermolecular interactions—attraction and repulsion—mostly converge. To generate manifold embeddings, MEMS utilized a non-linear method of stochastic neighbor embedding (SNE), NeRV (neighbor retrieval visualizer). The process preserved the local neighborhood of surface points between the manifold and embedding. The neighborhood was defined by pairwise geodesic distances among surface points of manifold (e.g., Hirshfeld surface or solvent-exclusion surface). The local electronic attributes on a molecular surface were then mapped to the manifold embedding and further featurized as numerical matrices to represent the quantum information. The attempt in solubility prediction demonstrated great potentials of utilizing MEMS of electronic attributes in chemical deep learning.

MKMS is a further enhancement to the MEMS process in which quantum molecular data is directly kernelized without the intermediate step of generating a feature matrix, thus preserving 100% of the data of the 3-dimensional molecular structure.

### BRIEF DESCRIPTION OF THE FIGURES

The above mentioned and other features and objects of this invention, and the manner of attaining them, will become more apparent and the invention itself will be better understood by reference to the following description of an embodiment of the invention taken in conjunction with the accompanying drawings, wherein:

FIGS. 1A-C show example equations for performing MKMS, where in FIG. 1A, Equation 1 provides mathematical basis for kernelization of 3-dimensional surface using a Gaussian Process, in FIG. 1B, Equation 2 yields a Spectral Mixture kernel expressed as a mixture of Gaussians, and, in FIG. 1C, Equation 3 defines an SM kernel based on a reduced-rank Sparse Gaussian Process on a surface.

FIG. 2 is a schematic diagrammatic flow diagram of the MKMS system according to one embodiment of the present invention.

FIG. 3 is an illustration of the differences between the precursor MEMS system and the MKMS system.

FIG. 4 demonstrates optimization of SGP regression kernels on ibuprofen ESP and f 2 surface, in which the originally calculated surfaces, or ground truth, are shown on the right.

FIG. 5A presents representative MKMS kernels calculated of (a) carbamazepine (C₁₅H₁₂N₂O), (b) flufenamic acid (C₁₄H₁₀F₃NO₂), and (c) sulfisoxazole (C₁₁H₁₃N₃O₃S), along with the respective ESP and ƒ² molecular surfaces viewed from two different angles.

FIG. 5B illustrates three schemes of selecting inducing points on SGP regression and kernel learning of ESP (a) and ƒ² (b) of danazol, including 52 vertices chosen to be closest to the respective atoms, 28 and 54 vertices determined by the landmarking method, and derived kernel matrices (c). The inducing points are marked by dark squares; the top three landmarks are indicated for the latter approach (#2 slightly blocked). The original values of ESP and f² are of ground truth; the same iso-surface is shown from two opposite views.

FIGS. 6A and 6D are histograms of the root mean squared error between MKMS predicted solubility values and true values from the Solubility Challenges and ESOL database, respectively. FIGS. 6B and 6E are histograms of log of MKMS predicted solubility values and the experimental results from the Solubility and ESOL database, respectively. FIGS. 6C and 6F are scatterplots of the MKMS predicted solubility values and the experimental results from the Solubility and ESOL database.

FIG. 7 is a schematic diagrammatic flow diagram of the precursor MEMS process.

FIG. 8 is high level process flow diagram of a precursor MEMS process.

FIG. 9 shows molecular mappings using various mapping methods in a precursor MEMS process.

FIG. 10 shows a progression of dimensionality reduction in several images of tolfenamic acid in precursor MEMS process.

FIG. 11 shows MEMS illustrations of polymorphs of tolfenamic acid.

FIG. 12 shows the Shape Context transformation in a precursor MEMS process.

FIG. 13 present are graphical results of training accuracy in a precursor MEMS process.

FIG. 14 shows MEMS representations generated by RBF interpolation (a) and SGP (b), respectively.

FIG. 15 is a series of graphs showing MEMS (shape context) of 162 latent molecules reduced to 3-D latent spaces by GPLVM.

FIG. 16 is a picture illustrating surface points on a MEMS representation.

FIG. 17 are pictures showing an RBF interpolation filter.

FIG. 18 are illustrations of dimensionality reduction of manifolds.

FIG. 19 provides both an illustration of tolfenamic acid conformers and MEMS representations.

FIG. 20 is a schematic process diagram one embodiment of the computational prediction system and example MEMS features input therein.

FIG. 21 is a schematic process diagram one embodiment of the computational de novo development system and example MEMS features input therein.

FIG. 22 is a schematic diagrammatic view of a network system in which embodiments of the present invention may be utilized.

FIG. 23 is a block diagram of a computing system (either a server or client, or both, as appropriate), with optional input devices (e.g., keyboard, mouse, touch screen, etc.) and output devices, hardware, network connections, one or more processors, and memory/storage for data and modules, etc. which may be utilized in conjunction with embodiments of the present invention.

FIG. 24 is a flow chart diagram showing and generation of example MEMS representation for input in a neural network.

FIG. 25 is a flow chart diagram showing generation of a data structure based on manifold embedding.

FIG. 26 is a dimensionality reduction MEMS representation of the Hirshfield surface of tolfenamic acid.

FIG. 27 is a MEMS representation of FIG. 20.

FIG. 28 is an illustration of two embeddings (ƒ2) of the same surface manifold being cut in an MEMS process, with the middle embedding being the uncut manifold.

FIG. 29 is an illustration of Shape-context matrices of MEMS (ƒ−) derived from the same surface manifold as FIG. 28.

FIG. 30 is an illustration of Heatmap and cluster analysis of EMD values.

FIG. 31 is an illustration of ESP and ƒ2 MEMS of four selected molecules used in the deep-learning prediction.

FIG. 32A-G is an illustration of prediction results of three separated sets of deep learning training data.

FIG. 33A-I is a series of graph charts showing prediction results from three separated sets of training data in an MEMS process.

FIG. 34A-F is a series of graph charts showing prediction versus experimental values by a closed MEMS process.

FIG. 35A-B provides the MEMS graphic representations of several compounds.

FIG. 36 illustrates predicted versus experimental solubility values in one data set of training according to an MEMS process.

FIGS. 37 and 38 show graphic projections of predictive versus experimental data relating to several molecules.

FIG. 39 is a graph chart of data distributions according to a MEMS process.

FIGS. 40-43 have bar and graph charts of prediction results of in an MEMS process.

FIGS. 44-49 show heat maps according to an MEMS process.

Corresponding reference characters indicate corresponding parts throughout the several views. Although the drawings represent embodiments of the present invention, the drawings are not necessarily to scale and certain features may be exaggerated to illustrate better and explain the present disclosure. The flow charts and screen shots are also representative in nature, and actual embodiments of the disclosure may include further features or steps not shown in the drawings. The exemplification set out herein illustrates embodiments of the present disclosure, in one form, and such exemplifications are not to be construed as limiting the scope of the invention in any manner.

### DETAILED DESCRIPTION

The following discussion is presented to enable any person skilled in the art to make and use the technology disclosed and is provided in the context of a particular application and its requirements as well as a particular system environment and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The recent advances in machine and deep learning (ML/DL) are poised to transform the “datarich and algorithm-poor” status quo of drug research to a new era of data-driven quests. The digital transformation is, however, deterred partially because most chemical information, manifested through molecular interactions, resides on high-dimensional manifolds rather than in a low-dimensional Euclidean space. In mathematics, a manifold signifies a space with unique topology in which the local neighborhood of a manifold point is approximated Euclidean; importantly, the local Euclidean metrics are collectively associated and defined globally by the topological metrics. For instance, a molecular surface mathematically is a 2-D manifold in 3-D space, and a surface point bears a 2-D tangent plane that characterizes its neighborhood metrics.

A molecular structure-property relationship may be regarded as molecules distributed on a manifold connected to the property of interest. Such properties may include solubility (a combination of hydrophobicity and lattice energy), developability, permeability, and physiochemical properties such as absorption, distribution, metabolism, excretion, and/or toxicity (ADMET). Moreover, most of the current molecular representations for ML/DL stem from the ball-and-stick notion of molecular structure, but they carry little information about molecular interactions, making data-driven drug research highly challenging. It is thus critical for developing ML/DL models to directly utilize chemical information, in particular, a molecule's quantum information (QI) that governs intermolecular interactions, to fend off the “curse of dimensionality” (COD), and to ensure manifold topography is correctly and fully observed by ML methods.

The precursor work to the present disclosure explored projecting electronic quantities on molecular surface to a lower dimensional embedding—Manifold Embedding of Molecular Surface (MEMS). The MEMS concept aligns with manifold learning and implements a non-linear method of stochastic neighborhood embedding (SNE). The process preserves the local neighborhood information of surface points between the manifold and embedding. To mitigate information loss, MEMS was derived from a cut manifold and could use multiple cut MEMS of the same molecule in DL. MEMS was featurized by Shape Context and utilized MEMS SC matrices as molecular representation to predict several molecular properties, including solubility. The solubility prediction performed significantly better than literature-reported efforts, which mostly utilized structural descriptors.

The present disclosure is drawing to Manifold Kernelization of Molecular Surface (MKMS), which provides further improvement to the manifold embedding concept. In contrast to MEMS, the MKMS utilizes kernel learning to directly capture the information of electronic attributes, such as electrostatic potential (ESP) or Fukui functions, on a molecular surface without resorting to the manifold embedding process. The essence of the present approach is to conduct Gaussian Process (GP) regression on a manifold. Because of its non-parametric nature, GP utilizes the covariances among training or existing data points to predict the distribution function at a new point (both mean and variance). The covariance matrix, or kernel, signifies the mutual relationships among the (training) data points. Various kernel functions are devised to define how two data points are related typically based on their distance and (trainable) hyperparameters. Sparse GP (SGP) utilizes a fraction of training or inducing points to fit all data points by optimizing the hyperparameters of the covariance matrix of the inducing points. The resultant covariance or kernel matrix thus encodes data relationships and their mutual influences among the inducing points, as well as the connections with all the training points.

Testing disclosed herein demonstrated that using dozens of spectral mixture (SM) kernel in SGP resulted in highly expressive kernels for a molecular surface. Notably, if applying SGP directly on a molecular surface, it would render the covariance matrix not semi-positive definite even if geodesic distances are used in kernel calculation. In that regard, a reduced-rank GP approach was adopted along with calculated covariances with eigen solutions of the graph Laplacian, resulting in kernels representing both electronic quantities and the topology of the molecular surface.

An artificial neural network (ANN) model was developed to predict molecular properties and utilize MKMS as a molecular representation in DL. As MKMS kernels are symmetric positive definite (SPD), SPDNet was adopted to maintain the underlying Riemannian topology by SPD matrices in data training. Self-attention in the ANN architecture was further utilized to ensure permutation-invariance in processing the kernel input. The supervised manifold learning model, dubbed SPDNET Attention, outperformed a previous model using MEMS as molecular input for predicting solubilities.

Examples of programming languages, code, and/or data libraries, and/or operating environments for use with the present technology include Python, Numpy, R, Java, Javasript, C#, C++, Julia, Shell, Go, TypeScript, and Scala.

### Methods—Manifold Kernelization

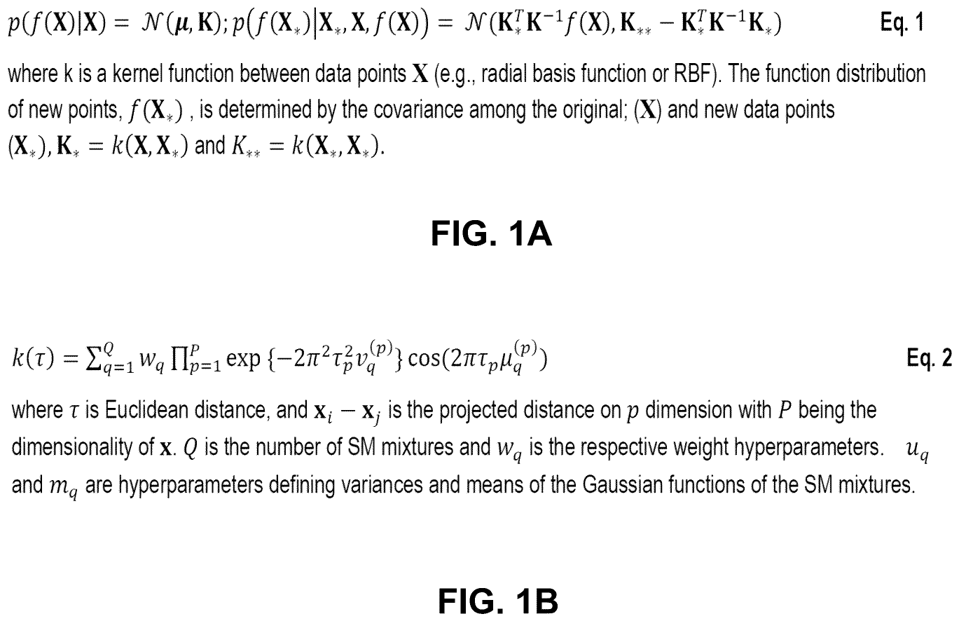

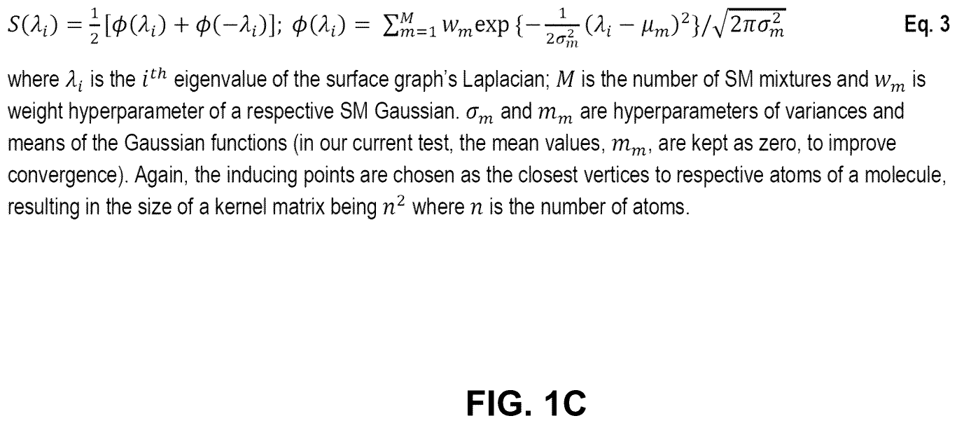

Extending the MEMS concept and its applications in predicting molecular properties, the manifold learning methodology as described herein is advanced by directly running kernel learning on a molecular surface. According to this approach, Sparse Gaussian Process (SGP) is performed with proper kernel functions on the electronic attributes of a MEMS or a surface manifold. SGP is an approximate GP approach given by Equation 1 as set forth in FIG. 1A and reproduced as follow:

$$p ( f ( X ) \vert X ) = 𝒩 ( μ , K ; p ( f ( {X}_{*} ) \vert {X}_{*} , {X}_{*} f ( X ) ) = 𝒩 ( {K}_{*}^{T} {K}^{- 1} f ( X ) , {K}_{**} - {K}_{*}^{T} {K}^{- 1} {K}_{*} ) \qquad Eq . 1$$

where k is a kernel function between data points X (e.g., radial basis function or RBF).

In Eq. 1, the complex 3-dimensional surface can be mathematically defined as a joint distribution (p(f(X)|X)), of a Gaussian function f(X) and X, where X are data points obtained from the surface. This covariance function can also be modeled as a Gaussian function (N(μ, K)) of the mean function of the surface (μ), and a covariance function of the surface (K). With sufficiently complex surfaces these functions cannot be exactly determined but can be estimated with a SGP, involving X*, a set of new data points derived from the surface. The mean function can be approximated as a product of K*TK−1f(X), where K* is K(X,X*). The covariance function can be approximated as the difference of K** and K*T K−1K*, where K** is K(X*, X*).

The hyperparameters used in the kernel functions are optimized iteratively as the SGP regression is performed. As in MEMS, Spectral Mixture (SM) is used as the kernel function, and is run directly on the embedding points in Euclidean space. The SM kernel is critical to this process, as it models the spectral density of the covariance function as a mixture of Gaussians and contains more trainable hyperparameters than alternative kernel functions like RBF or Matérn, as shown in Eq. 2.as set forth in Equation 2 of FIG. 1B, reproduced as follows:

$$k ( τ ) = {\sum }_{q = 1}^{Q} {w}_{q} {\prod }_{p = 1}^{P} exp ( \{ - 2 {π}^{2} {τ}_{p}^{2} {v}_{q}^{( p )} \} cos ( 2 {πτ}_{p} {μ}_{q}^{( p )} ) \qquad Eq . 2$$

where τ is the Euclidean distance between the true points and the selected points (xi−xj). τp is the projected distance on dimension p of with P, maintaining the dimensionality of x·q is an index of Q, the number of SM mixtures used in the analysis. Wq is the weight of the qth SM. Uq and mq are hyperparameters defining the variances and means of the Gaussian function of the SM mixtures.

In precursor MEMS a molecule's quantum information was modeled by embedding information from its molecular manifold to a 2-dimensional surface. This was necessary because a GP cannot be directly applied in a manifold by merely replacing point Euclidean distances with their geodetic distances. However, utilizing a recent development of reduced-rank GP, the covariance function of a spectral solution manifold can be solved using SM as the spectral density to generate GP kernels without going through the embedding process. The Graph Laplacian is calculated from a triangular mesh of the molecular surface and then is eigen-decomposed. By selecting the eigenvectors of 4 of the 5 smallest eigenvalues (excluding the smallest), an SM kernel can be created, as shown in Equation 3 of FIG. 1C, reproduced as follows:

$$S ( {λ}_{i} ) = \frac{1}{2} [ ϕ ( {λ}_{i} ) + ϕ ( - {λ}_{i} ) ] ; \qquad Eq . 3$$

$$ϕ ( {λ}_{i} ) = {\sum }_{m = 1}^{M} {w}_{m} exp \{ - \frac{1}{2 {σ}_{m}^{2}} {( {λ}_{i} - {μ}_{m} )}^{2} \} / \sqrt{2 {πσ}_{m}^{2}}$$

Where λi is the ith eigen value of the surface graph's Laplacian, M is the number of SM mixtures, and wm is the weight hyperparameter of a respective SM Gaussian. Σm and mm are hyperparameters of variances and means of the Gaussian functions. These values may be kept to 0 to improve convergence to singular values.

### Methods—Manifold Learning

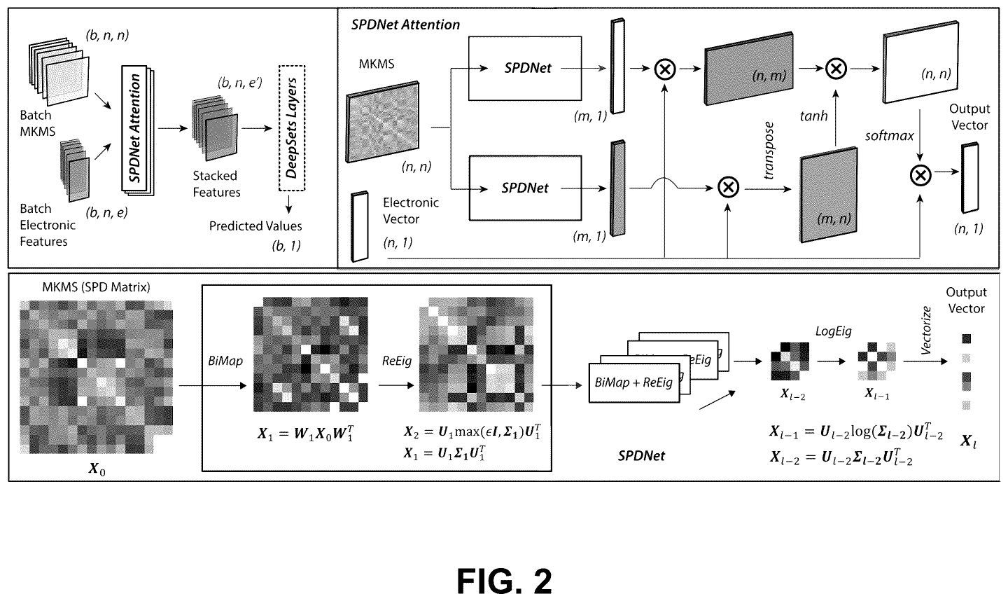

To utilize the MKMS kernel directly as a molecular representation for machine learning, an ANN model was developed to predict molecular properties. A kernel or covariance matrix is symmetric semi-positive definite; in practice, such a matrix is regularized by adding a small number (e.g., 1.0\hat{ }−7) to its eigenvalues, becoming SPD. Importantly, SPD matrices reside in a Riemannian manifold, and the topology of the Riemannian manifold needs to be respected by ML/DL models. SPDNet was therefore adopted, where three neural network operators, BiMap, ReEig, and Log Eig, aim to preserve the Riemannian manifold during training. In particular, BiMap layer achieves dimensionality reduction of an input SPD, ReEig regulates the learning (by replacing the smallest eigenvalues of an SPD with a predetermined cutoff parameter, generally, 0.00001 to 0.001), and log Eig projects an SPD from the Riemannian manifold to its Euclidean tangent space. The mathematics of these operators are shown in FIG. 2, which depicts an example artificial neural network model to predict molecular properties based on MKMS kernels of electronic quantities, including ESP and Fukui functions.

SPDNet Attention as described herein was developed to ensure permutation invariance of electronic feature vectors. In other words, exchanging i and j rows (and corresponding columns) of an MKMS kernel should not affect the prediction outcome. The attention algorithm follows the essence of self-attention or Transformer. Two SPDNets may be utilized to generate the “query” and “key” of the input electronic feature vector (i.e., ESP, ƒₚ, or ƒₙ of inducing points on a molecular surface). The query and key then multiply to form the weighting matrix and then mask the input vector by matrix multiplication. The weighted electronic vectors are stacked together, forming a feature matrix further processed by DeepSets layers. In solubility prediction tests, three electronic feature vectors were used for each molecule: (1) electrostatic potential (ESP), (2) nucleophilic Fukui function (ƒ⁺), electrophilic Fukui function (ƒ⁻), along with the respective SPD kernels, as input for the ANN model. By running through four layers of SPDNets Attention and stacking with the original electronic vectors, 15 feature vectors (3+3×4) were generated for each molecule, which were then fed into DeepSets layers.

Solubility prediction was conducted using the MKMS SPDNet Attention model. Two databases were utilized, including 200 molecules collected out of the First and Second Solubility Challenges, 42, 43 and 1128 molecules from ESOL, 44 in which 20 were excluded due to quantum mechanical computation difficulties by the basis set used in molecule optimization (out of the 20 molecules, 16 bear iodine and 4 sulfur). Each molecule was fully optimized with electronic quantities, including ESP, ƒ⁺, ƒ⁻, and dual descriptor of Fukui function (ƒ²), calculated by Gaussian (Gaussian, Inc., Wallingford CT) at the level of B3LYP/6-31G(d′,p′).

### Example 1—Manifold Kernelization

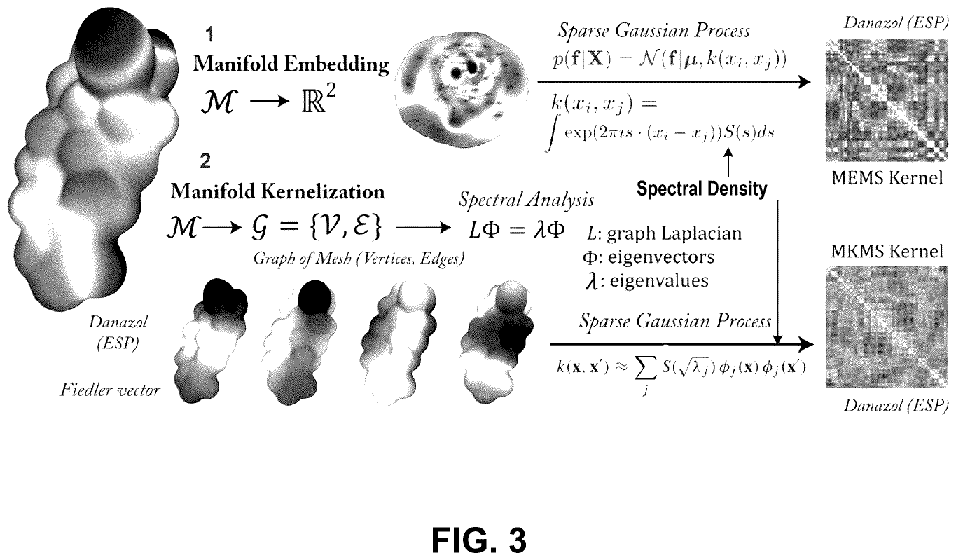

Kernelization was performed on the original MEMS maps and directly on molecular surfaces. FIG. 3 highlights the manifold kernelization principles. As illustrated, on an embedding map, kernels are derived by SGP regression using spectral mixture (SM) kernel functions. The illustrated ESP kernel of danazol was calculated with 96 SM functions. When directly conducted on a molecular surface, manifold kernelization was initiated by spectrum analysis of the manifold graph Laplacian followed by SGP regression with SM kernel functions. The exemplified danazol kernel was calculated with the top 640 spectral eigenvectors and 64 SM functions; the top 4 non-constant eigenvectors are shown (the first surface is the Fiedler vector). The two danazol kernels resemble each other, but the MKMS seems more detailed and refined. The improvement results from the completeness of considering the surface manifold in the kernel learning in the MKMS process, whereas MEMS suffers from information loss due to the embedding process that results in false positives and false negatives. Nonetheless, the similarity between the two still indicates that MEMS is capable of retaining dominant electronic features on a surface, enabling deep learning of molecular interactions and properties.

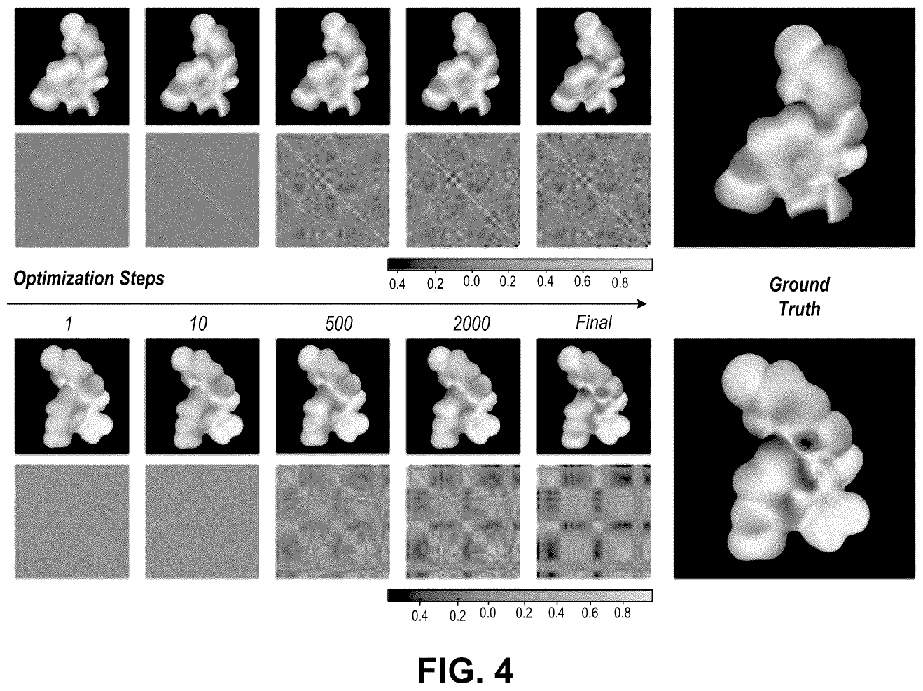

The manifold kernelization process is demonstrated in FIG. 4, where indomethacin surfaces of ESP and ƒ² are fitted by SGP regression. The reiterative steps optimized the global hyperparameters of kernel functions (Eq. 3, FIG. 1C), which constitute the covariance or kernel matrices. While the electronic quantities on the molecular surface already started to resemble the original values (ground truth) just after the first step, it took a few thousand steps for the kernels to finally converge. The electronic values at the inducing points dominated the initial regression steps, indicated by the largest values along the diagonal of the covariance matrix. As the optimization progressed, fitting errors declined with more off-diagonal values becoming dominant. Still, there are minor features of the ground truth or QM-calculated electronic values that the fitted surfaces could not fully reproduce. The minor blemishes stemmed from only 41 inducing points utilized in the SGP regression to process 5,497 vertices, which also suggested that electronic features on a molecular surface are smoothly distributed and governed by the underlying atoms.

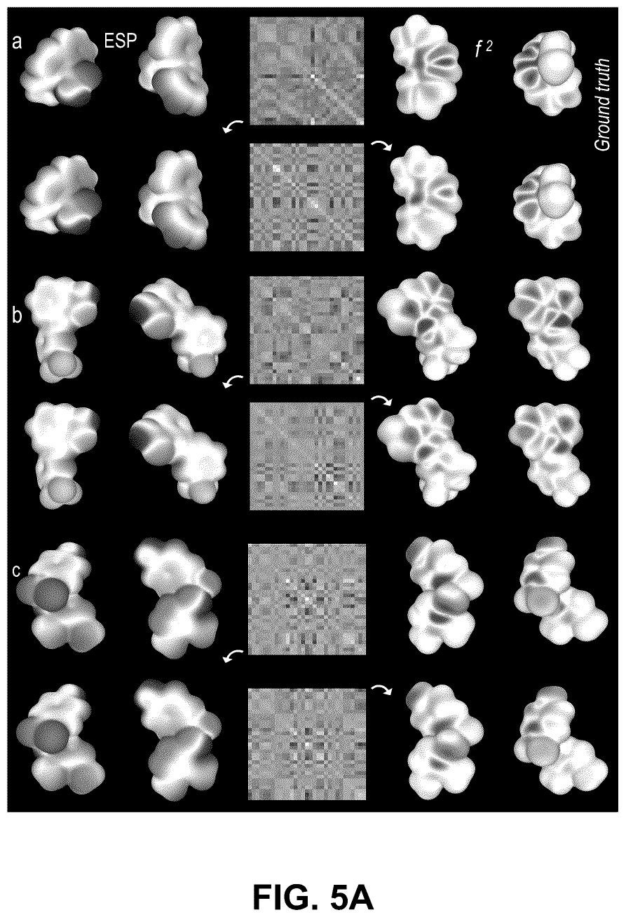

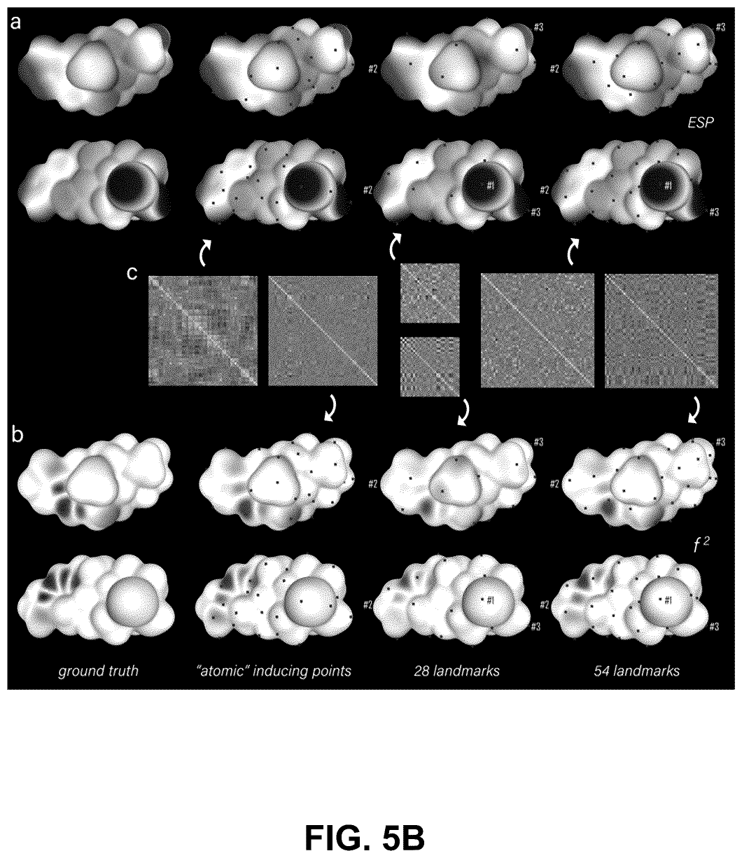

Several MKMS calculated of selected molecules from the Solubility Challenges are illustrated in FIG. 5A. In particular, representative MKMS kernels calculated of carbamazepine (C₁₅H₁₂N₂O; a), flufenamic acid (C₁₄H₁₀F₃NO₂; b), and sulfisoxazole (C₁₁H₁₃N₃O₃S; c) along with the respective ESP and ƒ² molecular surfaces viewed from two different angles. Of each molecule, the first row is of ground truth and the second row is of the finally regressed electronic values on the surfaces. The size of each kernel is n×n, where n is the number of atoms of the molecule. The kernels are highly distinctive among the molecules because have different surface topology and geometry; the electronic attributes also significantly vary, determined by the underlying molecular structure. As a covariance matrix, a kernel captures the interrelationships among the inducing points on the molecular surface, decided by electronic values and surface topological features. ESP and Fukui functions of each molecule share some similarities but are distinguishable. The resemblance apparently stems from the same inducing points on the molecular surface used to conduct SGP. From the machine learning perspective, MKMS differentiates molecules and, more importantly, encodes the quantum information of a molecule that is directly related to intermolecular interactions, both strength and locality. MKMS kernels are anticipated to ease the development of ML/DL models, making learning architecture design less complicated.

As shown in FIG. 5B, two different numbers of inducing points, 28 and 54, were selected for the kernel learning of danazol surfaces, in addition to the atomic scheme in which the molecule has 52 atoms and thus the same number of inducing points. Compared with the ground truth values of electronic properties (ESP and ƒ²), each scheme led to reasonably well-fitted surfaces. For ESP, electronic values vary smoothly and globally. They are closely fitted, especially for the dominant features at the —N—O— bond of the 5-membered isoxazole ring, the ethynyl, and adjacent hydroxyl groups. Comparably, Fukui functions (ƒ²) are more localized, notably around the shared bond between the isoxazole and its neighboring rings and can apparently benefit from more inducing points in such areas. Either method of using 52 atomic or 54 landmarks performs similarly. While it is overall satisfactory, using 28 points shows noticeable deficiencies.

The landmarking approach determines an inducing point by its variance value in the covariance matrix utilized in SGP. The covariance functions are calculated by eigenvalues and eigenvectors of the graph Laplacian of a surface mesh. In the present study, a molecular surface is triangulated, and its spectral properties are thereby determined by surface curvatures. As visually suggested in FIG. 5B, the inducing points are strategically positioned at curved spots. Positioning landmark points has little to do with the electronic values at these points or the rest of the (training) points because the Laplacian does not consider electronic properties and the full GP covariance matrix is evaluated with default hyperparameters. The 28 landmarks are mostly shared by the 54 landmarking exercise, as corroborated by the same top three ones marked in the figures. Subsequently, while the inducing points are identically selected between the two surfaces of ESP and ƒ², the covariance values of the points are further governed by the electronic values of these points, as well as the rest of the points. The hyperparameters of the kernel functions (Eq. 2, FIG. 1B) are learned by minimizing the fitting error in the electronic property between a regressed surface and the ground truth. Even for the pre-determined atomic inducing points, calculations of their kernel functions are eventually determined by their respective spectral properties and underlying electronic values. As such, MKMS essentially encodes the topology of a molecular surface and the data distribution of electronic values on the surface.

The MKMS kernels of the same electronic properties in FIG. 5B show similarities between the three selection approaches. Interestingly, the kernels remain expressive and globally comparable even for the case with 28 inducing points. This is supported by the fitted surfaces that highly resemble the ground truth. The finding is significant, as the landmarking approach facilitates effective, unsupervised dimensionality reduction of electronic features of a molecule. The significance justifies the prowess of supervised learning of the present disclosure, which essentially achieves guided dimensionality reduction by training data. Although only two landmarking schemes are shown, the results imply the continuous nature of MKMS with regard to dimensionality and, more importantly, robustness to preserve global features of local electronic distributions as a kernel dimension is reduced. In the predictions of solubility discussed herein, the original approach to choose “atomic” inducing points was utilized because of its simplicity and ostensible resemblance to a molecular structure, as well as its satisfactory performance in approximating the ground truth.

### Example 2—Manifold Learning

The electronic structures of the molecules were calculated from the Solubility Challenges and ESOL datasets. MKMS kernels were further derived from ESP and Fukui function quantities on each molecule's iso-electronic density surface (at 0.002 a.u.). In addition to solubility, SPDNet Attention may be trained to predict one or more of developability, efficacy, permeability, and ADMET properties using appropriate datasets, including the properties and data sources set forth in Table 1.

TABLE 1
Common Drug Developabilities and Available Databases

| Property | Data Source |
|---|---|
| water solubility | Solubility Challenges, ESOL |
| partition coefficient, logP | Martel, Eur J Pharm Sci 2013 |
| melting point | Salahinejad, J Chem Inf Model 2013 |
| sublimation energy | Chickos, Cryst Growth Des 2019 |
| membrane permeability | ChEMBL (Caco-2 cell lines) |
| BBB permeability | Meng, Sci Data 2021 |
| CYP 450 binding | PubChem (A1851) |
| microsomal clearance | ChEMBL (human microsomal) |
| microbiome activity | Javdan, Cell 2020 |
| HEK293 cell toxicity | NCATS OpenData |
| hPXR binding | PubChem (A1347033) |
| hERG inhibition | ChEMBL (ID#240) |
| P-glycoprotein binding | ChEMBL (ID#4302) |
| Drug-induced liver toxicity | FDA (DILIrank) |
| Tox21 screening data | tox21.gov |

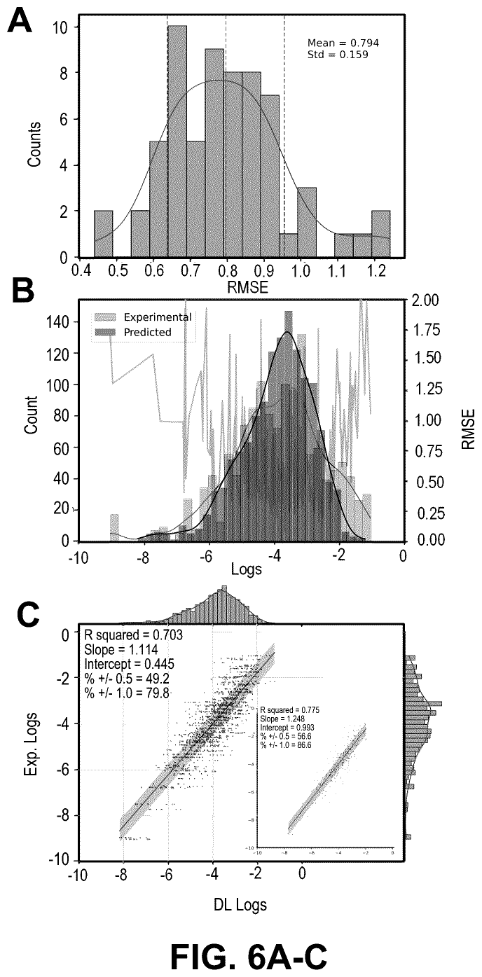

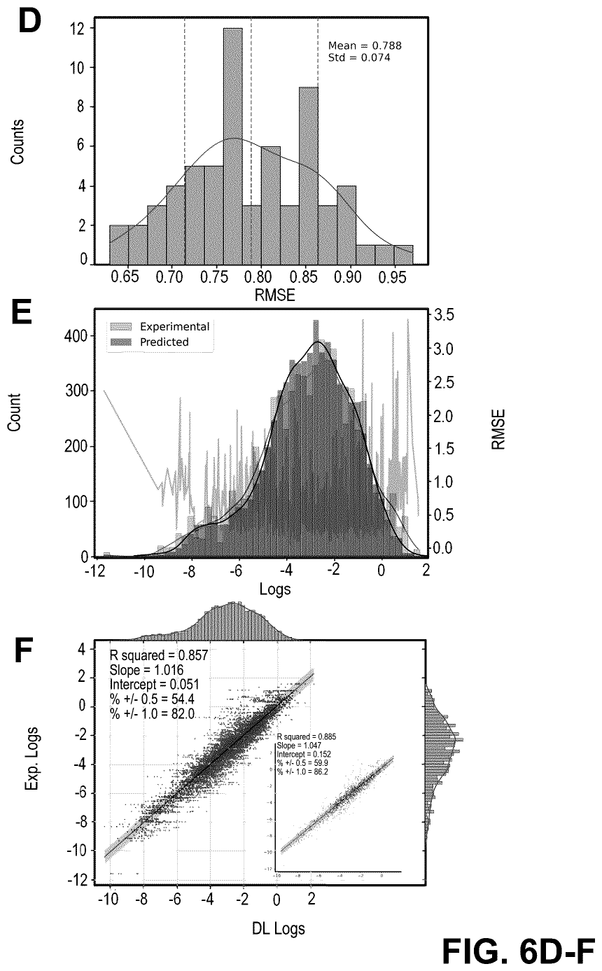

SPDNet Attention was used and trained using the solubility values from the datasets. Prediction metrics from 64 cross-validations of each dataset are shown in FIG. 6. In particular, FIG. 6 shows solubility prediction results by SPDNet Attention using the datasets from Solubility Challenges (a, b, and c) and ESOL (d, e, and f), including RMSE (a and d), distributions of the 14 predicted and experimental solubility values superimposed with RMSE values of individual molecules (b and e), and linear correlations between the predicted and experimental values with the averaged predicted values of each molecule shown in the insets (c and f). Each dataset was randomly split into training and testing by 90:10 to conduct 64 cross-validations.

The results highly resembled but outperformed previous results of using MEMS of the same solubility datasets. Because the number of training data or molecules was limited, especially when using the Solubility Challenges dataset, the prediction accuracy was greatly affected by how the dataset was split. A better prediction was typically obtained in the middle of the data distribution (b and e), where more training data points were available. Poor predictions appear at the two tails of the data distribution where few training data points are present. Importantly, the difference in prediction accuracy between the two datasets (e.g., shown by b and e) suggests that experimental errors of the Solubility Challenges dataset are significantly smaller than those in ESOL.

TABLE 2
Prediction Metrics by MKMS and MEMS.

| | MEMS SC Matrix | | | MKMS Kernels | | |
|---|---|---|---|---|---|---|
| | MAE | RMSE | R² | MAE | RMSE | R² |
| Solubility | 0.714 ± 0.145 | 0.892 ± 0.184 | 0.620 | 0.636 ± 0.128 | 0.794 ± 0.159 | 0.703 |
| Challenges | (0.546 ± 0.188) | (0.676 ± 0.243) | (0.767) | (0.512 ± 0.125) | (0.636 ± 0.157) | (0.813) |
| ESOL | 0.615 ± 0.049 | 0.815 ± 0.077 | 0.846 | 0.589 ± 0.054 | 0.788 ± 0.074 | 0.857 |
| | (0.552 ± 0.073) | (0.721 ± 0.107) | (0.879) | (0.536 ± 0.071) | (0.703 ± 0.097) | (0.884) |

As summarized in Table 2, the predictions by MKMS outperformed the best ones using MEMS. For example, prediction of the Solubility Challenges dataset with MEMS yielded the best RMSE of 0.892 (at 90:10 splitting of the dataset for training and testing); with MKMS, the best prediction resulted in the RMSE of 0.794. When the dataset was split at 95:5, the RMSE was significantly improved from 0.676 to 0.636. Significant improvements can also be seen in predicting ESOL. When the dataset splitting was 90:10, the average RMSE was 0.788, compared to 0.815 obtained in the previous effort of using MEMS. The RMSE was improved from 0.721 to 0.703 when the dataset was split at 95:5. The improvement by MKMS and associated ANN framework suggests that MKMS is highly expressive and more authentic than MEMS. There are at least two contributions that benefit from MKMS. First, MKMS captures the fullness of quantum information on a molecular surface (manifold) without loss of information due to the embedding process by MEMS. An MKMS kernel describes the distribution of electronic quantities and mutual relations among their scales and encodes the inherent manifold topology. In addition, an MKMS kernel is an SPD matrix, and the deep learning framework, SPDNet Attention, correctly maintains the topological features of the Riemannian manifold on which MKMS kernels of the molecules are distributed. The architecture further enables the permutation invariance of using SPD matrices as input and also implements the self-attention mechanism in learning. Test results using DeepSets to directly treat MKMS kernels led to much worse prediction results. DeepSets was utilized in the earlier work to predict solubility by MEMS shape-context matrices, but the ANN model described herein applies Euclidian metrics to process input matrices.

Accordingly, using MKMS kernels and SPDNet Attention is capable of achieving reliable outcomes to predict molecular properties. Prediction uncertainty would most likely stem from the quality of training data, not the model or molecular representation. It is further believed that MKMS kernel is robust against dimensionality reduction steps used in deep learning. In principle, reducing the dimension of a covariance matrix of SGP regression means using fewer inducing points. Under supervised learning, a smaller covariance matrix may retain the salient quantum information governing the property of interest.

A one non-transitory computer-readable medium (CRM) is disclosed that includes instructions that, when executed by at least one processor, cause the at least one processor perform an MKMS process. In one example the CRM may cause the processor to: (1) receive a dataset of electronic quantities across a manifold topology of a molecule of interest; (2) perform manifold regression on the electronic quantities to encode quantum information of the molecule in a covariant matrix (kernel), wherein the covariant matrix captures mutual relationships among the electronic quantities and the manifold topology; and (3) export the covariance matrix as a symmetric semi-positive definite matrix for use as an input to a neural network model configured to utilize the matrix for predicting molecular properties. Electronic quantities according to examples can include ESP and/or Fukui function quantities.

A molecular surface according to embodiments herein can be transformed using, for example, reduced-rank Sparse Gaussian Process (SGP) with Spectral Mixture (SM) as the kernel function. According to examples, the SGP utilizes a set of inducing points chosen as the closest vertices to the respective atoms of a molecule, resulting in a kernel matrix being n\hat{ }2, where n is the number of atoms in the molecule. In the same or other examples, the covariance function of the spectral solution of a manifold can be identified through an eigen decomposition of the Graph Laplacian of a molecular surface. In the same or yet other examples, the hyperparameters used in the reduced-rank SGP, particularly those associated with the means and variances of the Gaussian functions can be set to 0, to improve convergence. The kernel matrix can also be modified via eigen value reduction to a number near 0, generally between 0.001 and 0.00001, to allow for matrix or other value optimization. In one example, a combination of resulting kernel matrices, derived from different molecular surface from the same molecule, can be used to predict the molecule's chemical properties.

According to embodiment, dimensionality reduction may be used to project the data from a Euclidean tangent space or other similar space to a Riemannian manifold or other similar space. Projection can also be used to project the data from a Riemannian manifold or other similar space to a Euclidean tangent or other similar space.

Any neural network(s) (or other machine or deep learning models) configured and/or trained to receive a single positive definite (SPD) matrix or derivates thereof as input to predict an embedded molecule's chemical properties can be used consistent with the present disclosure, including, in one example, a trained SPDNet Attention model. According to examples, the model of embodiments of the invention involves a Deep Sets-based Graph and Self-Attention Network with input from a computational chemistry approach to evaluating electron density and solubility values as labels. As intermolecular interactions are very important in determining the solubility of a molecule and are represented well by electron density, electron density of the drug molecules may be used computationally to predict solubility.

The method of generating unique descriptors of intermolecular interactions in the precursor MEMS method was accomplished by: 1) using quantum mechanical conceptual density functional theory (CDFT) calculations to generate measures of electron density from crystal structures; 2) generating the molecular surface by determining the contribution of electron in the crystal structure from individual molecules; and 3) embedding the CDFT calculated values onto the three-dimensional (3D) molecular surface which is then projected into two dimensions (2D) using a stochastic neighborhood embedding approach. From each atom location in the 2D space, radially and angularly distributed electrostatic potential and Fukui function values are taken as input for the neural network.

The present method of generating unique descriptors of intermolecular interactions can be performed using MKMS. In one example, MKMS method includes the steps of: (1) receiving a dataset of electronic quantities across a manifold topology of a molecule of interest; (2) performing manifold regression on the electronic quantities to encode quantum information of the molecule in a covariant matrix (kernel), wherein the covariant matrix captures mutual relationships among the electronic quantities and the manifold topology; and (3) exporting the covariance matrix as a symmetric semi-positive definite matrix for use as an input to a neural network model configured to utilize the matrix for predicting molecular properties.

In the field of molecular modeling, the precursor MEMS process was aimed to represent a molecule as a chemically authentic and dimensionally reduced feature for computing molecular interactions and pertinent properties. It aligned with Manifold Learning by treating a molecular surface as a manifold and seeking its lower-dimensional embedding through dimensionality reduction. A molecular surface marks the boundary where intermolecular interactions-attraction and repulsion-mostly converge. It is well established that electronic attributes on a molecular surface, including electrostatic potential (ESP) and Fukui functions, determine both the strength and locality of intermolecular interactions. By preserving the spatial distribution of electronic quantities on a molecular surface to its 2-D embedding(s), MEMS represented a molecule with respect to its inherent chemistry of molecular interactions. Importantly, the underlying dimensionality of the electronic patterns on a molecular surface and its embedding is much smaller than that of MEMS. As the electronic structure and properties of a molecule are determined by its atoms and their relative positions, the true dimensionality of MEMS is similarly defined and may be uncovered by Shape Context (SC) or Gaussian Process (GP). In some embodiments, the derived GP parameters may be used to represent MEMS for deep learning. The dimensionality of MEMS features (by SC or GP) may be further reduced (e.g., Lawrence, N., “Probabilistic Non-Linear Principal Component Analysis with Gaussian Process Latent Variable Models” [Journal of Machine Learning Research 2005, 6, 1783-1816] or Kingma, D. P.; Welling, M., “Auto-Encoding Variational Bayes” [arXiv Preprint 2013, arXiv: 1312.6114]) to defeat the COD.

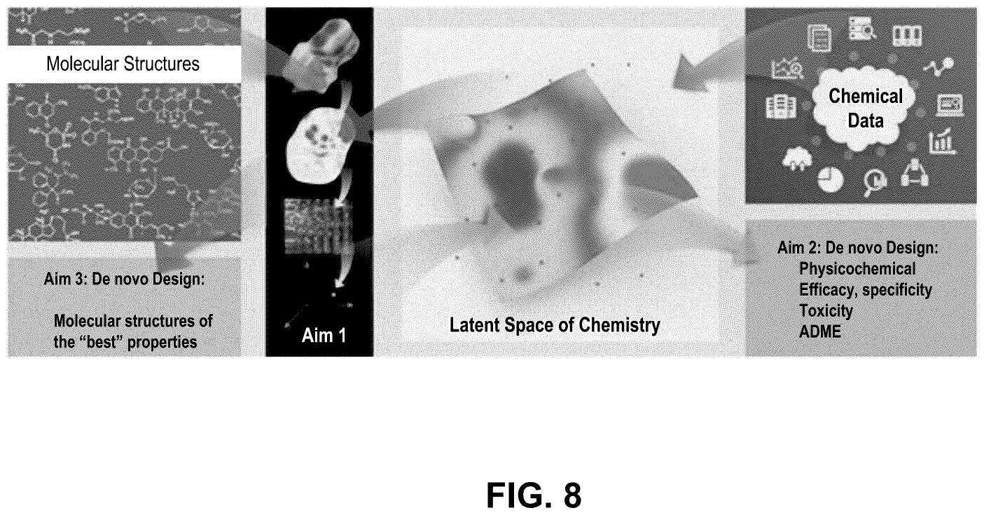

The significance of MEMS and its potential impact arises from the originality to featurize a molecule. By quantum mechanically “digesting” molecular structures, refining, and unifying the obtained information as manifold embeddings, MEMS consistently represented every molecule by preserving both the numerical values and spatial relations of the local electronic attributes on its surface. As it is mathematically derived from the local electronic properties of a molecule, MEMS may solely institute a unified quantum chemical space to potentially differentiate all molecules. As shown in FIG. 8, which shows a flow process which systemically unifying molecules in a low-dimensional, discriminative space of quantum chemistry by MEMS for the deep learning of molecular properties and de novo design of molecules and drug products the low-dimensional latent space, compared to that formed by conventional descriptors (and fingerprints), which may effectively and mutually bridge molecular structures and chemical properties. Sampling the space by respective datasets could uncover the underlying relationships of efficacy, toxicity, ADME (absorption, distribution, metabolism, and excretion) functionality, and product developability (solubility, physicochemical stability, mechanical strength, etc.). Importantly, having a structure-property relationship smoothly established in the latent space enables the optimization and discovery of the “best” MEMS of the property of interest. By reserving the manifold embedding process through generative deep learning, molecular structures of any given MEMS may be subsequently “recovered.” A huge amount of chemical data has been generated to date and is expected to accumulate at an accelerated pace, all of which may be converted to MEMS. Concurrently, deep learning has rapidly grown in recent years thanks to the exponential advance in computing hardware software and the demonstrated capabilities in potentially approximating any latent functions. By reducing electronic attributes on a molecular surface to 2-D embeddings as matrices, the power of tensor and GPU computing may be unleashed for chemical deep learning. The 2-D format also makes it straightforward to stack additional layers of information (such as MEMS of the conformers of a molecule). Embodiments of MEMS significantly advance datacentric drug research and fully utilize various data collections in drug discovery and development. By breaking COD, researchers in many areas may be unbridled to freely explore the chemical space and search for cures for diseases.

A molecular surface with electronic density and pertinent attributes is widely adopted in chemical studies to gain insights into molecular interactions, reaction mechanisms, and various physicochemical phenomena. However, it has never been attempted before to treat a molecular surface as a manifold, compute its lower-dimensional embedding(s), and preserve the electronic properties of the manifold embedding. MEMS provides a data structure and model of molecular representation for chemical learning to predict molecular interactions and physicochemical, biological, and pharmacological properties. The inspiration was borne out of the earlier studies of developing electron density-based quantities to characterize the locality and strength of intermolecular interactions. As the inventor attempted to match the local electronic quantities between two molecular surfaces and calculate the interaction strength, dimensionality reduction of molecular surfaces thus became a viable direction of pursuit with manifold learning and implemented a method of manifold embedding. Preliminary results reassure the groundwork that makes the MEMS concept truly distinctive: preserving the quantum chemical attributes on a molecular surface by a lower-dimensional embedding.

Because a molecular surface is enclosed, some surface points would fall in a “wrong” neighborhood when the manifold is reduced to a 2-D embedding. Cutting open a line on a surface can minimize the falsehood, except for points along the cutting line. The totality of information may be perceived by linearly combining the embeddings of several cuts as a final representation of the molecule. Similarly, linearly stacking the MEMS of a molecule's conformers (weighed by the respective conformational energies) carries additional information on the interaction specificity of the molecule when binding with flexible (or unknown) targets. By using manifold cutting and integrating the MEMS of conformers, the originality and broaden the applicability of the MEMS concept could be strengthened. Eventually, calculated MEMS libraries of molecules under various chemical environments may be provided to end users to utilize in their drug research.

More importantly, the low-dimensional features extracted from MEMS by SC and GP are further mapped to a lower-dimensional latent space by Gaussian Process Latent Variable Models (GPLVM) and Variation AutoEncoders (VAE). The latent space is essentially formed by quantum chemical dimensions that could serve as a singular universe to host every molecule discriminatively. As the mapping of a molecule to the latent space is achieved by considering probability distributions of MEMS features (via Bayes' Theorem), a functional surface could be truthfully (and smoothly) estimated or learned by using chemical data of the function (or property) of interest. The smooth function with the variance information could facilitate generative modeling of MEMS based on a molecular property, e.g., by Born-Oppenheimer approximation (BO). To reversely project a given MEMS to its causal molecular structures will be achieved by deep learning, in which the surface electronic features are connected to the covalent information of a molecule. The efforts create an entirely new avenue for the de novo design of molecules from the root of intermolecular interactions. Moreover, it is intriguing and potentially rewarding to investigate deterministic linkages between the GP (of a particular electronic attribute) on a molecular surface and the Gaussian-based atomic orbitals of the molecule. Such connection may advance chemical deep learning to a higher level, e.g., by directly approximating latent functions between molecular properties and the structure of a molecule without resorting to MEMS. Additionally, MEMS may become a holistic molecule representation widely adopted by in silico drug research. Because of its tensor format, MEMS is readily processed by computers. MEMS mitigates the loss of quantum chemical information of manifold embedding, integrates the information of conformational space of a molecule, further reduces the dimensionality of MEMS, and may develop further generative models of MEMS and molecular structures.

Drug discovery and development essentially revolves around assessing intermolecular interactions manifested as efficacy, toxicity, bioavailability, and developability. Molecular surfaces bearing electronic attributes, such as electrostatic potential (ESP), are often used to understand the strength of molecular interactions and, more importantly, the specificity or regioselectivity that are determined by local electronic structures and attributes, as well as by the spacing and alignment between the interacting molecules. Over the last several decades, the electronic attributes developed out of the Conceptual Density Functional Theory (CDFT) have proved insightful and predictive of reaction mechanisms and molecular interactions. Several essential attributes, including the Fukui function, are intimately connected with the Hard and Soft Acids and Bases Principle (HSAB). Being a local electronic perturbation-response quantity, Fukui function is directly proportional to the local softness or polarizability of a molecular system. It is defined as the partial derivative of the local electron density with respect to the number of electrons (N). Because of the discontinuity of N, Fukui function is further defined as nucleophilic (f+, due to increase in N) and electrophilic (f−, due to decrease in N) functions; the difference (f+−f−) is dual descriptor (f2). An outstanding region of Fukui function contributes considerably to the local and overall non-covalent interactions. Similarly, while an unambiguous solution is lacking for local hardness, ESP has been used for examining hard-hard interactions because it is capable of probing the local hardness. The inventor has exploited the local HSAB and developed several CDFT concepts to characterize the locality and strength of intermolecular interactions in organic crystals. The findings unveil that Fukui functions and electrostatic potential (ESP) quantitatively determine the locality and strength of intermolecular interactions, when examined at the interface between two molecules. In an organic crystal, such an interacting interface may be epitomized by Hirshfeld surface. One finding was that the electronic properties of the single molecule of interest—other than those of the explicitly interacted molecule pair—determine both the strength and locality of intermolecular interactions to be formed. That finding implies that the intrinsic electronic structure and local electronic attributes of an isolated molecule carry the inherent information about how the molecule interacts. Therefore, embodiments of the invention develop and apply CDFT concepts in drug research, especially the prediction of supramolecular packing and assembly and binding of small molecules with proteins.

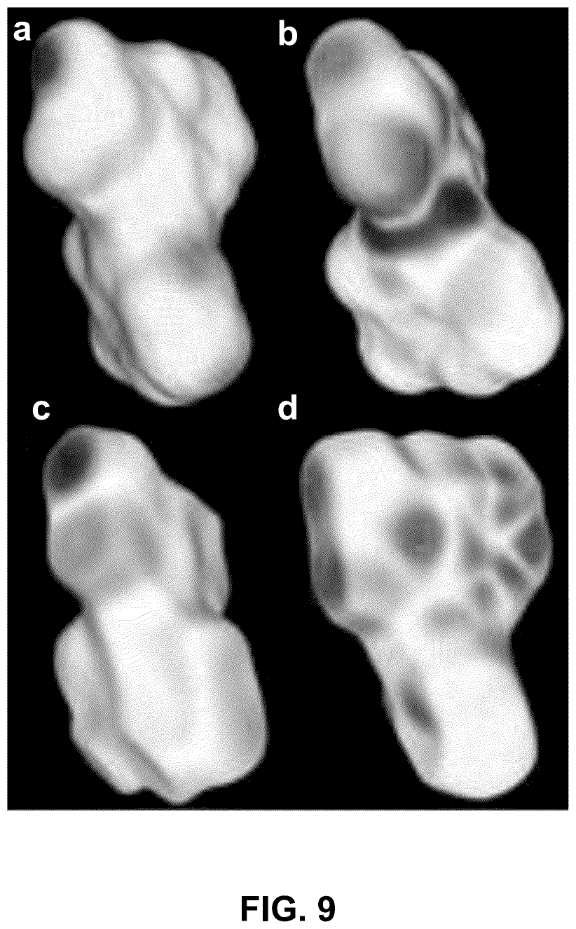

Several molecular surfaces are mapped by ESP and Fukui functions (ƒ²) from the studies are shown in FIG. 9, where solvent-exclusion surface (a & b) and Hirshfeld surface (c & d) of tolfenamic acid mapped with ESP (a & c) and ƒ² (b & d) are shown. The electronic patterns visibly indicate local electronic attributes of hardness and softness. A particular intermolecular interaction (e.g., hydrogen bonding or π-π stacking) is governed by the innate attributes at the contact area between the two molecules. An attempt was thus made to match the local hardness and softness of molecules to predict crystal packing and related physical properties. An attempt was also made to understand how a ligand molecule would fit into a protein pocket based on matching local electronic attributes between the ligand and protein surface. Nonetheless, the COD for either case was an issue as there are at least six degrees of freedom to arrange two molecules in space. This frustration has led to MEMS.

In addition, as the local electronic properties decide the interaction strength between two molecules, calculating the interaction energy directly from the local electronic values on the molecular surface(s) becomes advantageous. The challenge to identify feasible theories and mathematical functions led to a full embrace of neural networks. According to the Universal Approximation Theorem, any function could be approximated by neural networks. By developing a suitable network architecture and training it with data, the unknown function may be uncovered by approximation.

Treating a molecular surface as a manifold (specifically, Riemannian manifold), the MEMS concept roots in Manifold Learning. To generate manifold embeddings, a non-linear method of Stochastic Neighbor Embedding (SNE), Neighbor Retrieval Visualizer (NeRV), was implemented. The process preserves the local neighborhood of surface points between the manifold and embedding. The neighborhood is defined by pairwise geodesic distances among surface vertices of the manifold mesh (e.g., Hirshfeld surface or solvent-exclusion surface). The neighborhood is evaluated as the probability of vertex j in the neighborhood of vertex i:

$${p}_{j \vert i} = \frac{exp ( - {d}_{ij}^{2} / {σ}_{i}^{2} )}{{\sum }_{k \neq i} exp ( - {d}_{ik}^{2} / {σ}_{i}^{2} )} \qquad ( Eq . 4 )$$

where dᵢⱼ is the geodesic distance and σᵢ is a predefined hyperparameter of neighborhood coverage. A similar probability is defined by the Euclidean distance between the points i and j on the lower-dimensional embedding. Kullback-Leibler (KL) divergence is used as the cost function to optimize the latter probability distribution. Electronic properties on the molecule surface are pointwisely mapped to the MEMS.

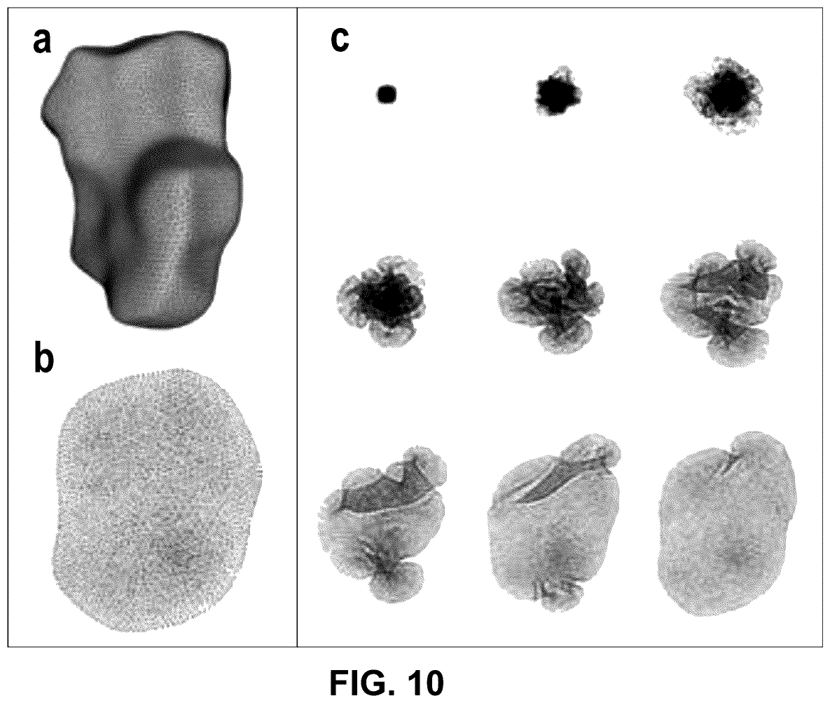

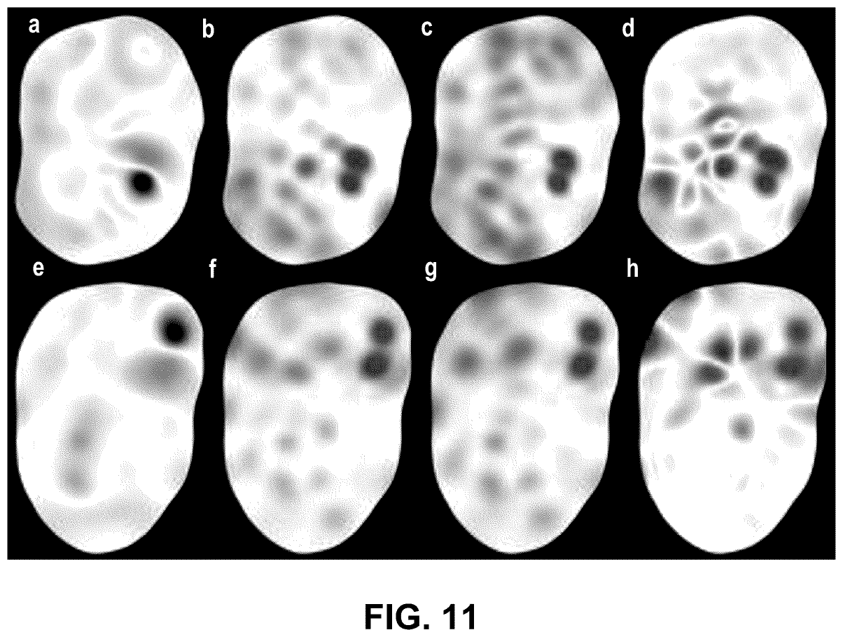

The dimensionality reduction process of a Hirshfeld surface of tolfenamic acid (metastable or Form II) is illustrated in FIG. 10, where dimensionality reduction of the Hirshfeld surface of tolfenamic acid (a) to its 2-D embedding (b) are shown as well as selected immediate steps of the KL optimization in (c). The surface was generated by Tonto, and the vertices were further optimized by isotropic remeshing in MeshLab56 (4a). Finally, the mesh vertices were input to the C++ program of KL optimization, producing 2-D points of MEMS (b). The optimization process is demonstrated in c, where the initially randomized points were progressively repositioned, finally reaching a local minimum of the cost function. FIG. 11 shows the interpolated MEMS coded with electronic properties on the corresponding manifolds. The electronic properties were calculated by Gaussian 09 of the respective single molecule, whose conformation was extracted from the crystal structure of Form I or II. The interpolation of electronic values was conducted by Gaussian-based radial basis functions (RBFs). Because RBF is a smooth function, it preserves dominant electronic attributes and smooths out minor features (false positives and negatives) on MEMS. The MEMS in FIG. 11 are of the same molecule but of different conformers, where dominant electronic properties and their spatial patterns seem to be preserved, more particularly where MEMS of two polymorphs of tolfenamic acid, Form II (a-d) and Form I (e-h) are shown having the electronic properties from left to right of ESP, ƒ⁺, ƒ⁻, and ƒ². The conformational difference is also truthfully reflected in the MEMS. Note that the scale in the MEMS in FIG. 11 is relative to the respective electronic attributes. Each image has its largest absolute value scaled to the full byte with positive numbers assigned to the red and negative to the blue channel, except for ESP where the red and blue are switched.

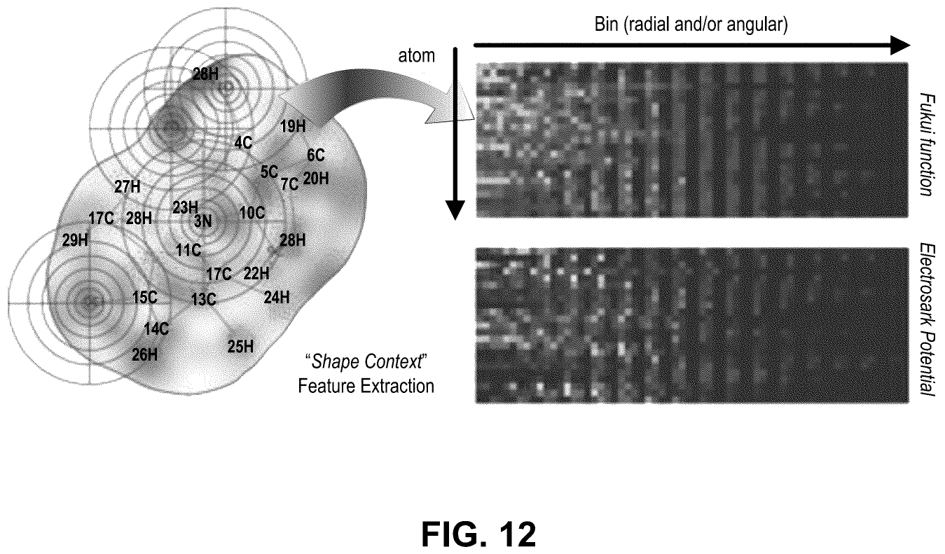

The intricacy of electronic attributes on a MEMS provides advantages to predict molecular interactions is by deep learning. It is possible to directly feed MEMS into the computer as an image and utilize CNN (convolutional neural network) for learning. Yet, the electronic pattern on a MEMS is relatively simple compared with real-life images typically used in CNN and seemingly comprised of overlapping 2-D bell-shaped functions centering around a few surface points. Embodiments of the invention provide a featurization method based on Shape Context in computer vision. Show in FIG. 12, where the shape contexts on four atoms are illustrated with the feature matrices are of the same molecule but different electronic properties, an SC feature matrix consists of rows of key points, which are the closest surface vertices to the respective atoms of the molecule in 3-D (denoted by atom indices on the figure). The intensities surrounding a key point on a MEMS image are spatially separated in predetermined bins along the radial direction. Each radial bin may be further divided into angular bins, where the angular direction is calculated against the geometric center to allow the rotational invariance of the feature matrix. Each row in the feature matrices (FIG. 12) comprises 16 radial bins, each of which has 4 angular bins. The SC images show the relative intensities of the bins, with the largest value scaled to the full byte. When a matrix is used in deep learning, it is the initially calculated values of the electronic properties that are processed.

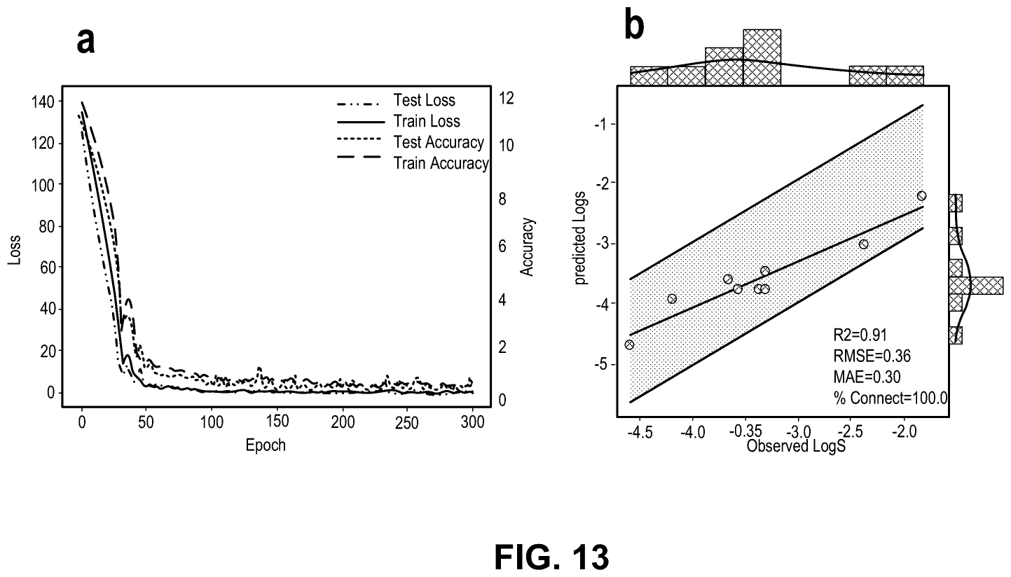

MEMS has been implemented for predicting water solubility of organic molecules by deep learning. The deep-learning effort utilized a curated dataset of about 160 molecules, which was split into 9:1 as the training and testing sets. Hirshfeld surfaces of the crystal structures of these molecules were calculated and reduced to manifold embeddings. Respective electronic properties (electron density, ESP, and Fukui functions) were evaluated of the single molecules with the conformations extracted from the individual crystals. Feature matrices were then derived by SC and used as the input for deep learning. The input of each molecule consisted of several feature matrices, including electronic density, ESP, ƒ⁺, ƒ⁻, and ƒ². DeepSets was adapted as the architecture of deep learning; self-attention was used as the learning mechanism in the deep neural network. PyTorch was used to implement the deep learning. The solubility prediction achieved a much-improved prediction accuracy compared with most of the reported literature studies. FIG. 13 shows one testing set's deep learning cost and prediction results where loss and accuracy of one test set of molecules during the deep learning (a) and predicted solubility (in log unit) vs. experimental values of the testing molecules (b). The mean absolute error (MAE) was 0.3 log unit, much smaller than the current prediction benchmark of 1 log unit. MEMS has also been used for predicting binding affinities of small molecules to cytochrome P450 enzymes. Computing the MEMS and SC features of more than 14,000 single molecules taken from PubChem (A1851), similar and better prediction results of classification (active vs. inactive) may be achieved compared to other reported efforts. Further analysis by regression prediction is also considered. Preliminary results support the feasibility of using MEMS of single molecules to predict intermolecular interactions.

The detailed descriptions which follow are presented in part in terms of algorithms and symbolic representations of operations on data bits within a computer memory representing genetic profiling information derived from patient sample data and populated into network models. A computer generally includes a processor for executing instructions and memory for storing instructions and data. When a general-purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self-consistent sequence of steps leading to a desired result. These steps are those requiring physical manipulations of physical quantities. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems and are not accessible except through sophisticated software systems. Data structures are not the information content of a memory, rather they represent specific electronic structural elements which impart or manifest a physical organization on the information stored in memory. More than mere abstraction, the data structures are specific electrical or magnetic structural elements in memory which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein which form part of the present invention; the operations are machine operations. Useful machines for performing the operations of the present invention include general purpose digital computers or other similar devices. In all cases the distinction between the method operations in operating a computer and the method of computation itself should be recognized. The present invention relates to a method and apparatus for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions or alternatively may be a higher level coding of the instructions that is interpreted to obtain the actual computer code. The software module may also include a hardware component, wherein some aspects of the algorithm are performed by the circuitry itself rather as a result of an instruction.

The present disclosure also relates to an apparatus for performing these operations. This apparatus may be specifically constructed for the required purposes or it may comprise a general purpose computer as selectively activated or reconfigured by a computer program stored in the computer. The algorithms presented herein are not inherently related to any particular computer or other apparatus unless explicitly indicated as requiring particular hardware. In some cases, the computer programs may communicate or relate to other programs or equipment through signals configured to particular protocols which may or may not require specific hardware or programming to interact. In particular, various general-purpose machines may be used with programs written in accordance with the teachings herein, or it may prove more convenient to construct more specialized apparatus to perform the required method steps. The required structure for a variety of these machines will appear from the description below.

The present invention may deal with “object-oriented” software, and particularly with an “object-oriented” operating system. The “object-oriented” software is organized into “objects”, each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects.

Messages are sent and received between objects having certain functions and knowledge to carry out processes. Messages are generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access. One feature of the object-oriented system is inheritance. For example, an object for drawing a “circle” on a display may inherit functions and knowledge from another object for drawing a “shape” on a display.

A programmer “programs” in an object-oriented programming language by writing individual blocks of code each of which creates an object by defining its methods. A collection of such objects adapted to communicate with one another by means of messages comprises an object-oriented program. Object-oriented computer programming facilitates the modeling of interactive systems in that each component of the system may be modeled with an object, the behavior of each component being simulated by the methods of its corresponding object, and the interactions between components being simulated by messages transmitted between objects.

An operator may stimulate a collection of interrelated objects comprising an object-oriented program by sending a message to one of the objects. The receipt of the message may cause the object to respond by carrying out predetermined functions which may include sending additional messages to one or more other objects. The other objects may in turn carry out additional functions in response to the messages they receive, including sending still more messages. In this manner, sequences of message and response may continue indefinitely or may come to an end when all messages have been responded to and no new messages are being sent. When modeling systems utilizing an object-oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object-oriented programming makes simulation of systems of interrelated components more intuitive, the operation of an object-oriented program is often difficult to understand because the sequence of operations carried out by an object-oriented program is usually not immediately apparent from a software listing as in the case for sequentially organized programs. Nor is it easy to determine how an object-oriented program works through observation of the readily apparent manifestations of its operation. Most of the operations carried out by a computer in response to a program are “invisible” to an observer since only a relatively few steps in a program typically produce an observable computer output.

In the following description, several terms which are used frequently have specialized meanings in the present context. The term “object” relates to a set of computer instructions and associated data which may be activated directly or indirectly by the user. The terms “windowing environment”, “running in windows”, and “object-oriented operating system” are used to denote a computer user interface in which information is manipulated and displayed on a video display such as within bounded regions on a raster scanned video display. The terms “network”, “local area network”, “LAN”, “wide area network”, or “WAN” mean two or more computers which are connected in such a manner that messages may be transmitted between the computers. In such computer networks, typically one or more computers operate as a “server”, a computer with large storage devices such as hard disk drives and communication hardware to operate peripheral devices such as printers or modems. Other computers, termed “workstations”, provide a user interface so that users of computer networks may access the network resources, such as shared data files, common peripheral devices, and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis. A “module” refers to a portion of a computer system and/or software program that carries out one or more specific functions and may be used alone or combined with other modules of the same system or program.

The term “desktop” means a specific user interface which presents a menu or display of objects with associated settings for the user associated with the desktop. When the desktop accesses a network resource, which typically requires an application program to execute on the remote server, the desktop calls an Application Program Interface, or “API”, to allow the user to provide commands to the network resource and observe any output. The term “Browser” refers to a program which is not necessarily apparent to the user, but which is responsible for transmitting messages between the desktop and the network server and for displaying and interacting with the network user. Browsers are designed to utilize a communications protocol for transmission of text and graphic information over a worldwide network of computers, namely the “World Wide Web” or simply the “Web”. Examples of Browsers compatible with one or more embodiments of the present invention include the Chrome browser program developed by Google Inc. of Mountain View, California (Chrome is a trademark of Google Inc.), the Safari browser program developed by Apple Inc. of Cupertino, California (Safari is a registered trademark of Apple Inc.), Internet Explorer program sold by Microsoft Corporation (Internet Explorer is a trademark of Microsoft Corporation), the Opera Browser program created by Opera Software ASA, or the Firefox browser program distributed by the Mozilla Foundation (Firefox is a registered trademark of the Mozilla Foundation). Although the following description details such operations in terms of a graphic user interface of a Browser, the present invention may be practiced with text-based interfaces, or even with voice or visually activated interfaces, that have many of the functions of a graphic based Browser.

Browsers display information which is formatted in a Standard Generalized Markup Language (“SGML”) or a HyperText Markup Language (“HTML”), both being scripting languages which embed non-visual codes in a text document through the use of special ASCII text codes. Files in these formats may be easily transmitted across computer networks, including global information networks like the Internet, and allow the Browsers to display text, images, and play audio and video recordings. The Web utilizes these data file formats to conjunction with its communication protocol to transmit such information between servers and workstations. Browsers may also be programmed to display information provided in an extensible Markup Language (“XML”) file, with XML files being capable of use with several Document Type Definitions (“DTD”) and thus more general in nature than SGML or HTML. The XML file may be analogized to an object, as the data and the stylesheet formatting are separately contained (formatting may be thought of as methods of displaying information, thus an XML file has data and an associated method). Similarly, JavaScript Object Notation (JSON) may be used to convert between data file formats.

The terms “personal digital assistant” or “PDA”, as defined above, means any handheld, mobile device that combines computing, telephone, fax, e-mail and networking features. The terms “wireless wide area network” or “WWAN” mean a wireless network that serves as the medium for the transmission of data between a handheld device and a computer. The term “synchronization” means the exchanging of information between a first device, e.g. a handheld device, and a second device, e.g. a desktop computer, either via wires or wirelessly. Synchronization ensures that the data on both devices are identical (at least at the time of synchronization).

Data may also be synchronized between computer systems and telephony systems. Such systems are known and include keypad-based data entry over a telephone line, voice recognition over a telephone line, and voice over internet protocol (“VoIP”). In this way, computer systems may recognize callers by associating particular numbers with known identities. More sophisticated call center software systems integrate computer information processing and telephony exchanges. Such systems initially were based on fixed wired telephony connections, but such systems have migrated to wireless technology.

In wireless wide area networks, communication primarily occurs through the transmission of radio signals over analog, digital cellular or personal communications service (“PCS”) networks. Signals may also be transmitted through microwaves and other electromagnetic waves. At the present time, most wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”), personal digital cellular (“PDC”), or through packet-data technology over analog systems such as cellular digital packet data (“CDPD”) used on the Advance Mobile Phone Service (“AMPS”).

The terms “wireless application protocol” or “WAP” mean a universal specification to facilitate the delivery and presentation of web-based data on handheld and mobile devices with small user interfaces. “Mobile Software” refers to the software operating system which allows for application programs to be implemented on a mobile device such as a mobile telephone or PDA. Examples of Mobile Software are Java and Java ME (Java and JavaME are trademarks of Sun Microsystems, Inc. of Santa Clara, California), BREW (BREW is a registered trademark of Qualcomm Incorporated of San Diego, California), Windows Mobile (Windows is a registered trademark of Microsoft Corporation of Redmond, Washington), Palm OS (Palm is a registered trademark of Palm, Inc. of Sunnyvale, California), Symbian OS (Symbian is a registered trademark of Symbian Software Limited Corporation of London, United Kingdom), ANDROID OS (ANDROID is a registered trademark of Google, Inc. of Mountain View, California), and iPhone OS (iPhone is a registered trademark of Apple, Inc. of Cupertino, California), and Windows Phone 7. “Mobile Apps” refers to software programs written for execution with Mobile Software.

“Machine Learning,” “Artificial Intelligence,” and related terms which relate to “Deep Learning” involve using data sets in a convolutional neural network/machine learning environment, wherein various quantum chemistry features of molecules are classified by using training and validation sets. Convolutional neural network architectures, for example without limitation, DenseNet, Inception V3, VGGNet, ResNet and Xception, may be configured for use with MEMS data structures and models. Typically, detection and identification systems are implemented in 3 phases consisting of data collection, development of the neural network, and assessment/re-assessment of the network. Development of the neural network involves selecting the optimal network design and subsequent training the “final” model, which is then assessed using the unseen test and validation sets. Training of the neural network is an automatic process, which is continued until validation loss plateaus. Training may be augmented with additional data sets and model manipulations. Coding may be implemented, for example without limitation, using the Python programming language with the Tensorflow and Keras machine learning frameworks. Training may be performed on GPUs such as made by nVIDIA Inc. of Santa Clara, California.

FIG. 12 illustrates the featurization scheme of Shape Context on MEMS. The process is aimed to reduce the dimensions of MEMS from the number of pixels of the 2-D embedding to the number of bins per keypoint on the embedding. The scheme produced advantages in the preliminary studies, corroborating that electronic density and properties on a molecular surface do not typically bear complex patterns but center on a few surface points near the closest atoms of the molecule. Additionally, transitions between major electronic spots on a molecular surface are smooth. The molecular surfaces in FIG. 9 exemplify the global electronic patterns.

Accordingly, analytical functions are presented that express MEMS and utilize the parameters of the functions to represent MEMS for deep learning. The radial basis function (RBF) interpolation method used in generating MEMS figures of electronic properties allows for recovering major electronic patterns on a 3-D molecular surface, with Gaussian Process as Gaussian kernels being used in both approaches. While RBF interpolation relies on finding the mixing weights of several Gaussian kernels (and often supplemented by polynomials) on scattered data points, GP is much more flexible and powerful to sample an infinite number of points as random functions that share a joint, underlying probability (Gaussian) distribution. Moreover, because the electronic properties of a molecule are collectively defined by the atoms and their chemical bonds, GP is highly appealing to capture the chemical intuition by describing the distribution of the electronic properties. To curb COD, GPLVM is further used to collectively reduce the dimensionality of a set of MEMS in a low-dimensional latent space.

GP of MEMS: Being a functional, GP regulates the mean and covariance functions as the normal distribution. Interpolation of the embedding points of MEMS may be treated as GP regression through the Bayes' Theorem. Briefly, given N training data points (X, Y) (i.e., the likelihood)—where X is the position vector, and Y is the mean value vector—the values at N_{*} testing position X_{*} (the posterior) are estimated by Y_{*}=K_{*}^{T}K⁻¹Y, where K_{*} is the N×N_{*} covariance matrix among the testing and training data points, and K is the N×N covariance matrix among the training data points. As the value at a data point is treated as a Gaussian, the variance at each testing point is estimated by the covariance matrices. Moreover, each element in the covariance matrices is a kernel function, and the Gaussian kernel (also known as squared exponential kernel or RBF kernel) is commonly used:

$$κ ( {x}_{i} , {x}_{j} ) = {σ}^{2} exp ( - \frac{1}{2 {l}^{2}} {( {x}_{i} - {x}_{j} )}^{T} ( {x}_{i} - {x}_{j} ) ) \qquad ( Eq . 5 )$$

where σ and l are predetermined hyperparameters controlling the vertical variance and smoothness of the GP at Y_{*}. Each kernel is determined by the distance between two data points. The mean at a testing position is a weighted regression by means of the training data; the kernel functions determine the weights (and thus the transition smoothness among the data points). Note that in MEMS, xᵢ is a two-dimensional position vector, but GP can handle multi-dimensional data.

Thus, the electronic properties of manifold embedding points can be interpolated (e.g., FIG. 10) with GP and produce full MEMS figures (e.g., FIG. 11). In this case, the embedding points of surface vertices are the training data (X, Y) and interpolating positions are the testing X_{*}. Nevertheless, there are typically more than 6000 surface points used in the manifold embedding process, making the GP calculation computationally demanding (mainly due to the inversion operation of K, which becomes intractable for larger N). Moreover, using a significantly large number of GP parameters (of the training data) to express the generally simple electronic patterns on a molecular surface is unnecessary. It deviates from the purpose of using GP parameters to represent MEMS. For this, sparse GP (SGP) was used instead.

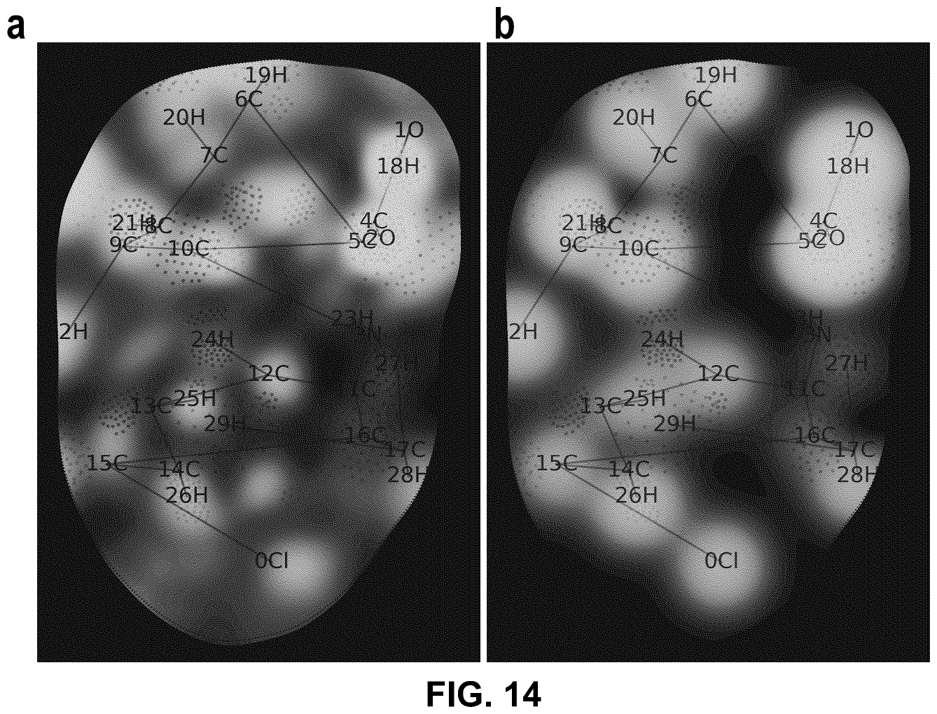

In SGP, rather than using the full training data points, a limited number of “inducing” points are selected, Xₘ. Then, the optimal Yₘ and Kₘₘ (covariance matrix of the inducing points) can be found by minimizing the KL divergence between the approximate and true Y of the training data. Mean values of the testing data are given as follows: Y_{*}=K^{T}*ₘ K⁻¹ₘₘYₘ, similarly to the regular GP. In this case, the key points were used as with SC (FIG. 12), i.e., for each atom, the closest vertex on the molecular surface was selected as an inducing point. The number of Xₘ is then equal to the number of atoms. KL divergence optimization was conducted to derive Yₘ and Kₘₘ with the embedding points as Y. FIG. 14, showing MEMS generated by RBF interpolation of all embedding points (a) and approximated MEMS by SGP (b), illustrates a preliminary attempt at using SGP to interpolate a MEMS. The result supports the feasibility and suggests several areas to explore.

In FIG. 14, the isotropic Gaussian kernel was used with the same length parameter, l, to calculate the distance along the x and y axes between two points (Eq. 5). The same parameter was used for all inducing points. In setting up this molecular model, the parameters in different directions and identify optimized l (and σ) by variational inference. The hyperparameters may be eventually linked to the underlying atomic type and bonding for each including point.

There are “spots” between inducing points (including the spots near the boundary) that were not picked up. These spots are likely due to unique chemical bonding, such as aromatic conjugation. To accommodate, different types of kernel functions and their combinations may be used.

As discussed above, MEMS of a closed molecular surface results in false negatives and positives. A substantial spot on a surface may lead to more than one spot on the MEMS. For dealing with a closed surface, it is worth exploring at least two points for each atom on the 2-D embedding. Rotational invariance is implicitly ensured because of the GP kernels are distance-based.

Thus, the parameters used to express MEMS are used as input for deep learning. At least three parameters representing each inducing point (two position numbers and mean value) of one electronic property. As discussed above, a few more parameters may be included to consider the anisotropic and in between patterns. Compared to the 64 bins used in FIG. 13, using the GP parameters considerably reduces the dimensionality of MEMS. Hyperparameters, including l and σ that are established according to the atomic and bonding information, could further reduce dimensionality (by using the atomic number to replace kernel parameters). Interpreting MEMS with SGP may potentially establish the connection between MEMS and the underlying chemistry (and molecule), facilitating the reserve mapping from MEMS to molecular structure.

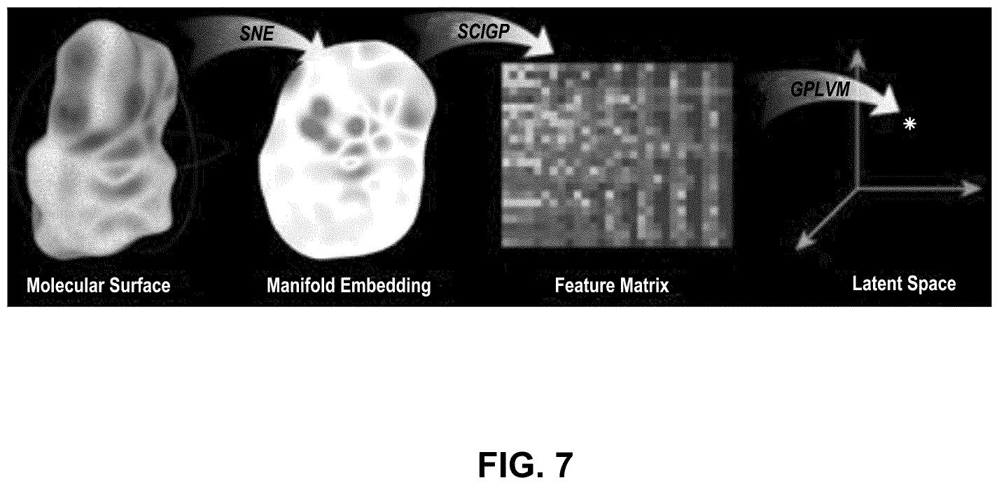

GPLVM of MEMS FEATURE: Even with the featurization of SC and GP, the dimensionality of MEMS remains significant. As indicated by FIG. 7, the aim was to reduce MEMS lastly-whether it is signified as an image (e.g., FIG. 11), SC matrix (e.g., FIG. 12), or GP parameter matrix-into a low-dimensional latent space. The dimensionality reduction may be implemented by GP Latent Variable Model. The method is not targeted to one MEMS, but a collection of MEMS calculated from a dataset of molecules.

GPLVM was developed out of the probabilistic principal component analysis (PCA). Each data dimension is treated as a GP of the (unknown) latent variables, and all the independent GPs are collected and optimized to derive the latent variables (and hyperparameters). Let Y∈ [symbol] be n MEMS feature matrices with p dimensions and X∈ [symbol] n latent variables with d<<p that are mapped to Y by GPs. Specifically, the latent variables are derived by maximizing the marginal likelihood defined below:

$$P ( Y \vert X , Φ ) = {\prod }_{j = 1}^{p} \frac{1}{{( 2 π )}^{\frac{n}{2}} {\vert K \vert}^{\frac{1}{2}}} exp ( - \frac{1}{2} {y}_{j}^{T} {K}^{- 1} {y}_{j} ) \qquad ( Eq . 6 )$$

where yⱼ is the j^{th} dimension of the data, K is the covariance matrix by X, and Φ are hyperparameters used by kernel functions.

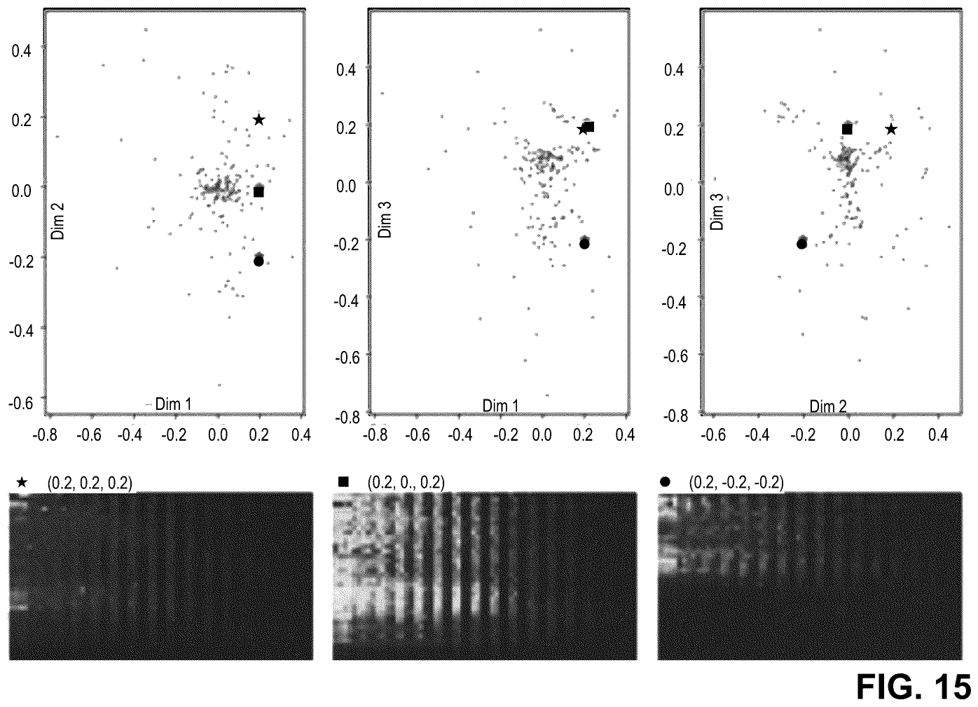

FIG. 15 illustrates the latent variables with three dimensions (d=3) of 162 MEMS feature matrices of negative ESP and electrophilic Fukui function (ƒ⁻), respectively, from the solubility prediction study (FIG. 13). In FIG. 15, MEMS (shape context) of 162 molecules are reduced to 3-D latent spaces by GPLVM, wherein the top row is based on ESP; the bottom on ƒ⁻ (three 2-D projections of the latent spaces are shown respectively). Each SC matrix has a dimension of 2304 (36 atoms times 64 bins; matrices of molecules with fewer atoms are padded with zeros) and is reduced by GPLVM to a single point in the latent space. It is evident that even with d=3, the high-dimensional MEMS are individually differentiated and uniquely signified in one chemical space. In this case, each molecule may be represented by only six dimensions (or 12 when positive ESP and ƒ⁺ are included). The feat is remarkable in chemical learning, as this allows for systemically projecting molecules in a low-dimensional space defined by quantum chemical dimensions. Given that SGP can be used in GPLVM, it is practically feasible to reduce millions of molecules (as MEMS) to a discriminative latent space. Moreover, because GPLVM is a generative model, any point in the latent space may be derived back to MEMS, opening a door for de novo design.

In one embodiment, a variational inference approach is used to optimize kernel parameters (Φ) and latent dimension before obtaining the latent variables (X). In addition, a Bayesian scheme to predict the latent variable for a new data (MEMS matrix) after the GPLVM latent space is established. This procedure improves property prediction when a (deep learning) model is trained with available data, and maximizes the extent to which the chemistry is captured by manifold embedding.

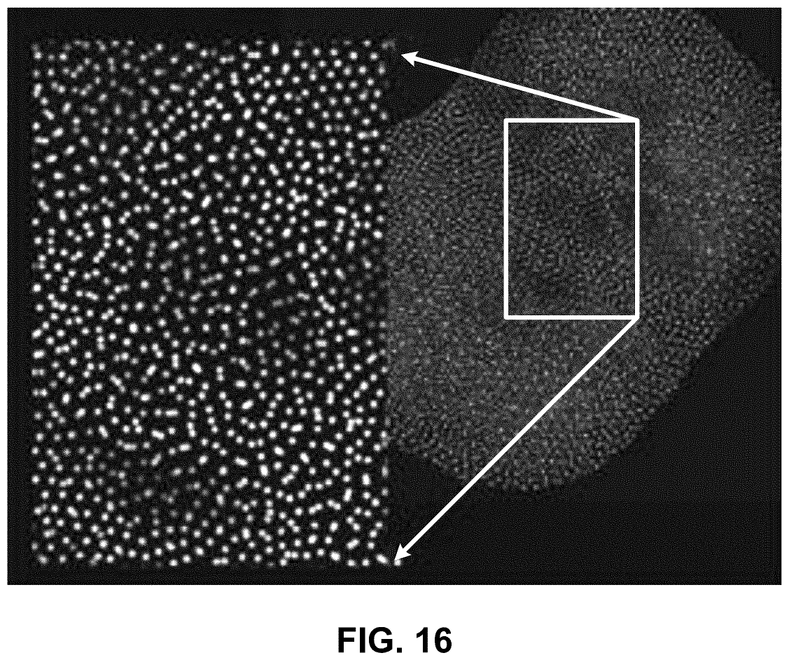

In FIG. 16 a molecular surface is enclosed, showing surface points on MEMS with the electronic property being ƒ². When calculating the geodesic distance between two surface points (or vertices of surface mesh), the shortest one is used in defining the neighborhood of a point (Eq. 4). However, the KL optimization process generates some false positives (distant neighbors in 3-D put in the same neighborhood on MEMS) and false negatives (near neighbors in 3-D separated on MEMS) on a final manifold embedding. This is illustrated in FIG. 16, where several blue, red, and white points are projected near each other in the highlighted area. On the original molecular surface, these points belong to different regions of ƒ² values. The ambiguity raises several questions in general for deep learning. First, how much information is lost by the dimensionality reduction process? Second, how to eliminate the generated false information and still be able to utilize the remaining data for prediction? Last, how to minimize the generation of false positives and negatives?

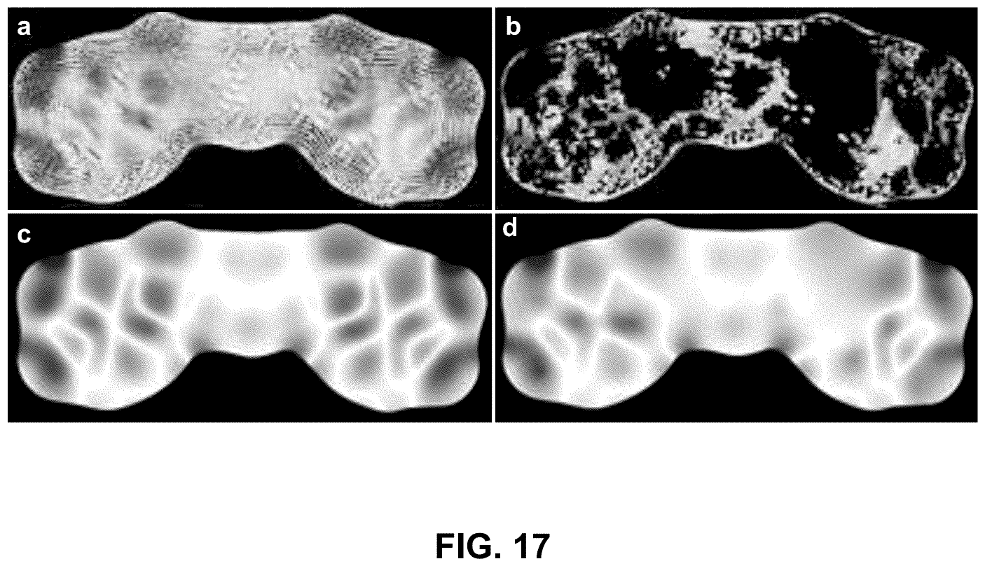

To address the first question, a basic scheme is used to estimate the percent of surface points in a “wrong” neighborhood. By defining the neighborhood size twice larger than the shortest inter-point distance on the surface or MEME, a point may be assigned as “outsider” if none of its MEMS neighbors comes from its neighborhood on the 3-D surface. Typically, the percent of outsiders is in the range of 20-40%, depending on the geometry of the molecular surface (and initial positions for KL optimization). For the second question, by using RBF to interpolate the points on MEMS and generate final MEMS images, the major or dominant electronic patterns and their spatial relationships from the original molecular surface are preserved on MEMS. FIG. 17 highlights one extreme case where 3247 points were kept out of 9011 embedding points but still led to the recovery of major electronic patterns by RBF interpolation. Ostensibly, these results suggest that the Gaussian kernels used by RBF smooth out data “noises” of false positives and negatives. Deeply, the recovery of dominant electronic features on MEMS implies a rationale in the inventive process: the underlying dimensionality of electronic attributes on MEMS is much smaller than that of MEMS itself (as a matrix or image format). The rationale is not intuitively surprising, as electronic features on a molecular surface smoothly spread over domains that are commensurate with the size of an atom.

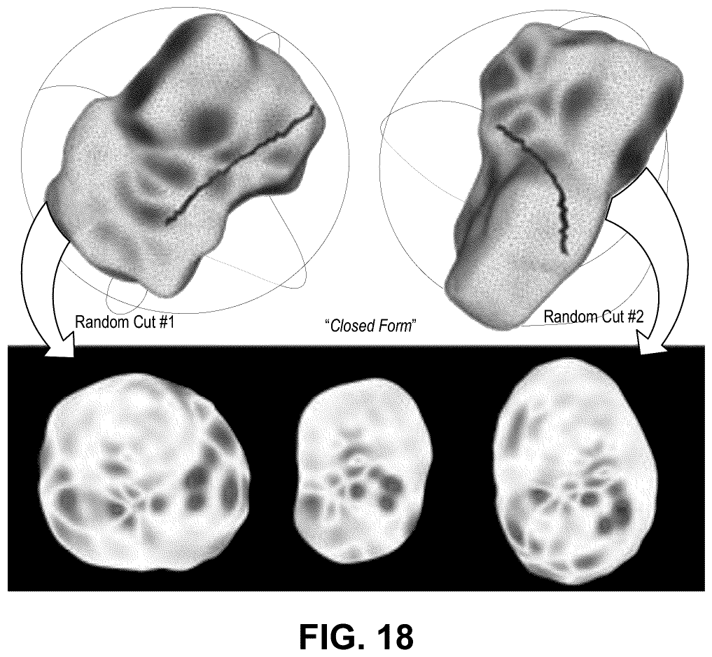

The ambiguity due to false positives and negatives may still result in uncertainties in deep learning of chemical data. This may be exemplified when the Earth globe is projected onto a World map—the “Far East” on one map becomes the “Central Kingdom” on another. To minimize the false information in MEMS, one method involves cutting a molecular surface by removing the connectivity between surface vertices along a randomly chosen surface line. The cutting forces the vertices to be the boundary points of MEMS while keeping other surface points in the right neighborhood. For example, FIG. 18 shows MEMS of the same surface (ƒ²) randomly cut, where both open-cut MEMS have no point in a wrong neighborhood, showing dimensionality reduction of the same manifold being cut with the middle embedding being of the closed manifold. In comparison, the closed MEMS has about 40% false points. The results are exciting and significant, supporting that the manifold embedding truthfully preserves the neighborhood information (of the cut surface). The closed MEMS still retains a good portion of major electronic distributions. Nonetheless, the cutting was done randomly and may not lead to the maximal preservation of the quantum chemical information on a surface. Another method that may be used is Geodesic Principal Analysis (PGA) to choose a surface direction for cutting. In brief, PGA firstly identifies an “intrinsic mean” on the surface of interest that has the minimal total geodesic distances to all surface points. Then, the surface points are projected to the tangential plane at the mean, and PCA is done on the projected points. Lastly, the identified principal vectors on the tangential space are projected back to the surface (manifold) as the principal geodesics. In this case, two orthonormal principal vectors will be yielded from the Euclidean space (on the tangential plane) and, subsequently, two principal geodesics on a molecular surface; by surface cutting along the principal geodesics and generate respective MEMS. The first principal geodesics may be a better choice of cutting as surface points collectively have larger variances along the direction. A molecule may be represented using both open-cut MEMS in deep learning.

Furthermore open-cut MEMS may be used for solubility prediction of organic molecules. To enhance the reliability of solubility prediction, cutting is done not randomly but between two surface points intercepted by principal axes of mesh points. As revealed in FIG. 13, the prediction accuracy was well under 1 log unit, a state-of-the-art standard currently observed by the community. Further improvement by using MEMS of cut surfaces may be achieved. In addition, the same deep learning framework with MEMS may be used to predict the membrane permeability of molecules. In addition to solubility, permeability is another key quantity determining the bioavailability of a drug molecule. In initial work, more than 3,000 Caco-2 permeability measurements from ChEMBL have been collected. Then curating the dataset, calculating electronic properties on these molecules' surface, and obtaining MEMS of both cut and closed surfaces may be used, featurized by SC and GP, as the input for deep learning. Moreover, low-dimensional latent representations of these MEMS may be derived by GPLVM and further exploited for deep learning as well. Lastly, MEMS of major conformers of each molecule (see below) may be tested for permeability prediction.

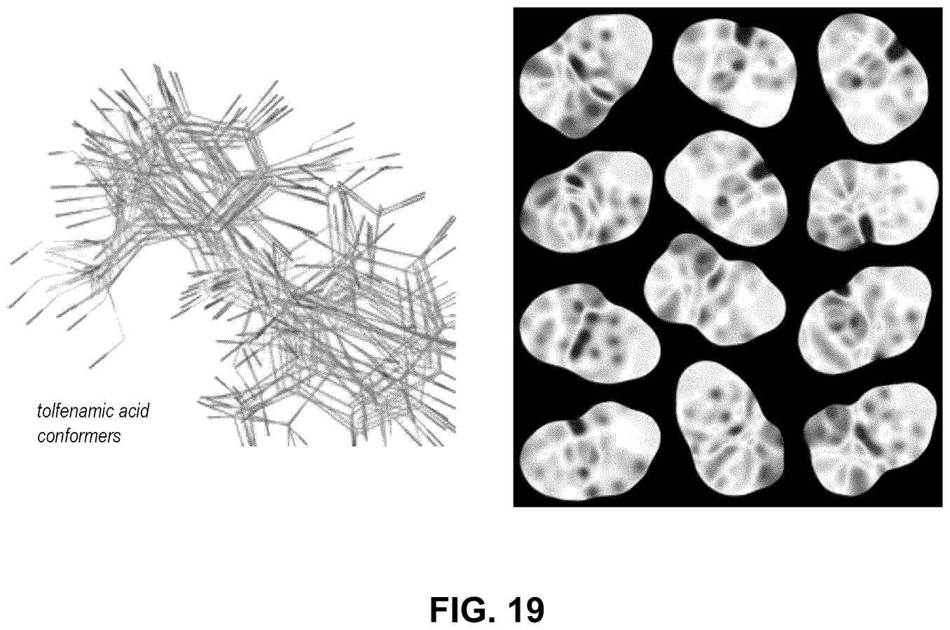

In preliminary studies, only either the most stable conformer (optimized by Gaussian 09 in vacuum or implicit solvent) or the conformer taken directly from the crystal structure of a molecule have been considered. This approach works well for molecular properties where such conformers are most relevant (e.g., solubility being a crystal property). However, the conformational flexibility of a molecule may also be considered for predicting molecular interactions such as protein-ligand binding, where the conformational energy is a co-factor. Because MEMS is already in 2D and readily featurized to a mathematic matrix (by SC or GP), integrating MEMS of the major conformers of a molecule is straightforward and improves the predictive power of a deep learning model significantly. FIG. 19 shows MEMS of the major MEMS of the molecular surfaces (ƒ²) of major conformers of tolfenamic acid, generated by ConfGen (Schrödinger Co.). Thus, stacking the conformers' feature matrices as the molecular representation is made in several embodiments of the invention. Conformational energies may be further used to weigh or normalize the respective matrices. This enables much-improved prediction results for studying molecular interactions and pertinent properties.

In one embodiment, prediction utilizes the conformer's MEMS for predicting binding activities to cytochrome P450 enzymes (CYP450). Preliminary studies were conducted with a publicly available dataset on PubChem (A1851), which reports binding assay results of molecules with six isoforms (1A2, 2B6, 2C9, 2C19, 2D6, and 3A4). The reported activity score is in the range of [0, 100], with the cutoff of 40 indicating a compound being active or inactive against a CYP450 enzyme. By selecting drug-like molecules, 14,567 unique molecules were identified from the database and trained the present deep-learning models. The most stable conformer of each molecule was identified and calculated by Gaussian 09; electronic properties on the surface were mapped to its MEMS and featurized by SC. The classification prediction (activity vs. inactive) showed comparable and better results compared with the literature-reported data. For example, the F-score was 0.87, and the accuracy was 0.79 for 1A2. On the other hand, the regression prediction resulted in mediocre MAE (mean absolute error) between 10 and 20 of activity scores. Much-improved prediction, especially the regression to have the MAE less than 10, may be achieved by considering the conformational flexibility of the molecules. By generating and selecting a predetermined number of major conformers of a molecule and calculating their MEMS, predictive quality improves. In addition, predictions of human microsomal clearance may be accomplished by using a curated dataset (>5,300 data points).

Chemical information determining both the strength and specificity of molecular interactions are carried by the molecular representation for predicting ligand binding with a protein. In the classification prediction of CYP450 binding, the MEMS used (of closed surfaces and without conformers) performed satisfactory, likely due to the binding strength being decided by the dominant electronic attributes that are retained in MEMS. However, for the regression prediction, complete information is helpful, especially about the spatial distribution of the electronic properties, along with the conformational dimension.

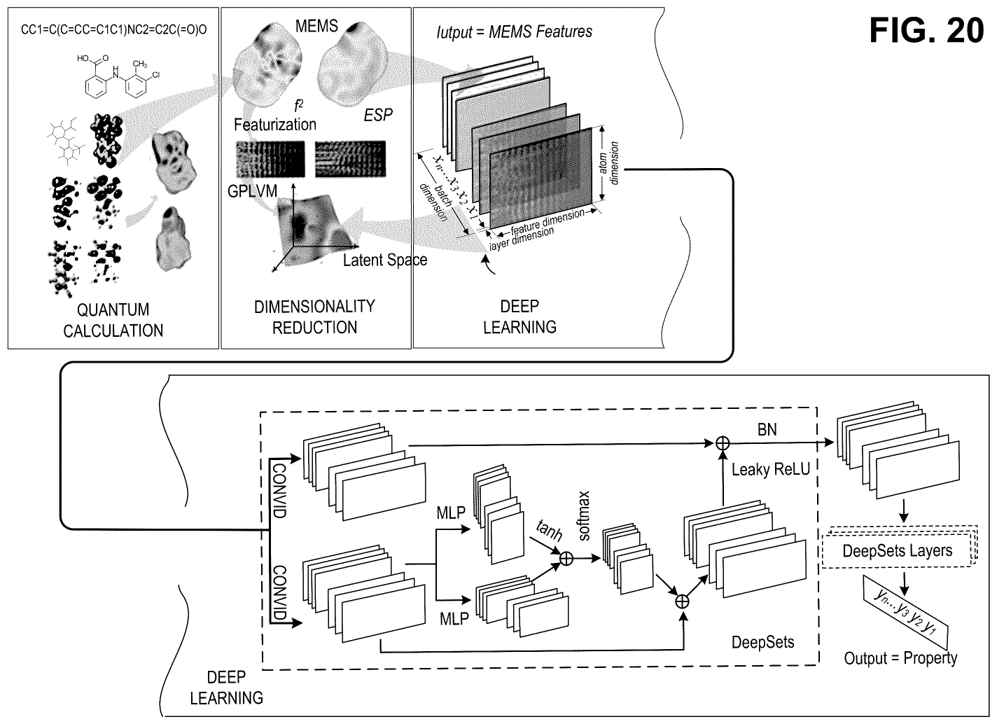

FIG. 20 shows one embodiment of a prediction framework of chemical properties (including affinity with protein target) integrating three parts: quantum calculation, dimensionality reduction, and deep learning. The process begins with obtaining a given molecule's conformer(s), followed by electronic calculation and generation of the molecular surface(s) of local electronic attributes. MEMS is then derived and featurized by SC or GP. Feature matrices of training molecules are projected to a low-dimensional space by GPLVM. MEMS features or latent variables of the training molecules are input to the deep learning module, in which DeepSets is adopted as the architecture. The input consists of a batch of molecules, each of which has several MEMS feature matrices of electronic properties. Multiple DeepSets layers are utilized, leading to the output of the network. With the training data of the property to be predicted, backpropagation is conducted by stochastic gradient descent leading to the optimization of network parameters.

Virtual screening applications of small molecules may be developed using the MEMS data structure and model. The general workflow of deep learning for the applications is highlighted in FIG. 20. These applications are summarized in Table 3. While the Universal Approximation Theorem suggests that artificial neural networks may approximate or learn any function, it does not reveal how a neural network is implemented. The performance of a deep learning model is governed by the network architecture, definition of the loss function, and quality of the training data. Equally important is whether the input contains sufficient information but with low-dimensional features to capture the underlying function. In a typical chemical learning of molecular property or function, the input is of a batch of molecules, in which each variable consists of feature vectors of the molecule's constituent atoms. Such a variable is a set where the ordering of atomic elements should be irrelevant for its functions to be learned. In this exemplary embodiment, DeepSets has been adopted as the deep-learning architecture. It is permutation invariant and potentially may learn any set function by a sum-decomposition scheme. In brief, the input is first transformed to a latent space—in this exemplary case, it is done by CONV1D along the feature dimension—and before transforming back to the output, the latent variables (or tensors) are processed by sum (or mean or max) operations to remove (or retain) the ordering information of the input set. Specifically, the self-attention approach was utilized as the sum-decomposition that is first proposed in Set2Graph. The attention architecture (FIG. 20) may be summed as:

$$\sum ( X ) = softmax ( \frac{tanh ( {f}_{1} ( X ) ) \cdot {{f}_{2} ( X )}^{T}}{{d}^{1 / 2}} ) \cdot X \qquad ( Eq . 7 )$$

where X is the input set, d is the feature dimension of X divided by a predetermined number (typically 10), and ƒ₁ and ƒ₂ are the query and key functions of self-attention, which are implemented by MLP or multilayer perceptron in the present models. Notably, the self-attention mechanism can be easily shown to be permutation invariant and is widely used to capture the intra-correlations of the input features, making DeepSets suitable choice for the present tasks. Additionally, regularization of each DeepSets may be done by batch normalization (BN) and Leaky ReLU; weight decay is also considered in the PyTorch optimizer to further mitigate model overfitting.

Table 3 of the Appendix lists the deep-learning applications made possible by embodiments of the present invention. The selection of the applications is not meant to be comprehensive but aimed to test the MEMS concept thoroughly. The properties include a single physical process (e.g., dissolution), binding to a single protein target (e.g., CYP450), permeating through the cell membrane, or undergoing much more convoluted processes such as cell-based target-binding. The DILI database is curated from the drug labeling and clinical observations, presenting a challenging case for deep learning due to data noises and multiple in vivo events leading to DILI. A specific implementation involves carefully going through each database and focusing on drug-like molecules (neutral and with molecular weight <600 Da).

In one embodiment, completing the deep-learning exercise may take up to one year of computational time with conventional research computing resources. On a typical computer node with 20 cores, it takes less than 10 minutes to do the quantum calculation and dimensionality reduction (i.e., the first two modules shown in FIG. 20) of a single molecule. Both Gaussian 09 and the C++ programs of manifold embedding are fully parallelized; the process of launching and processing the input and output of various programs is fully streamlined by shell scripts. With 24 nodes, one may create the MEMS features of more than 10,000 molecules per week. Implementing deep learning by PyTorch with CUDA; for example, in the classification prediction of CYP450 binding, it typically takes less than 4 hours on an NVIDIA A100 GPU to process >12,000 molecules with about 11 million neural network training parameters of 9 DeepSets layers. Thus, the deep-learning projects listed in Table 1 may take two years by typical research group.

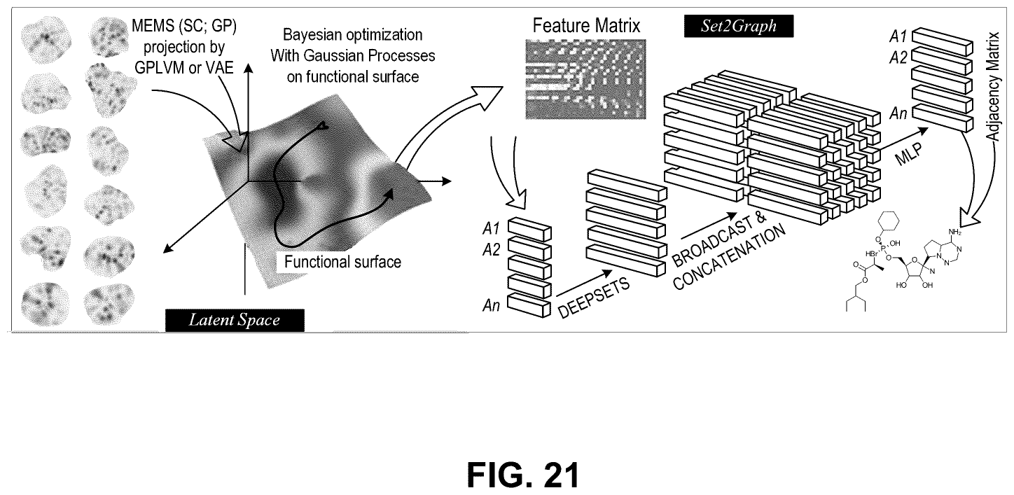

FIG. 21 illustrates the process and system of de novo development. Two components of the de novo design framework are shown: MEMS as feature matrices are firstly projected to a latent space by GPLVM or VAE; Bayesian optimization of the latent variables regarding a particular property is conducted to identify the best MEMS features; and a deep learning model based on Set2Graph is established to predict the adjacency matrix of a molecule from a given MEMS feature matrix.

GPLVM may be used to project MEMS feature matrices of molecules, which are generated by SC or GP, to a latent space. MEMS as an image (e.g., in FIG. 18) may also be projected directly, but its true dimensionality is much smaller than that of the image itself. Much fewer GP parameters of a MEMS may be capable of fully representing the electronic properties on a molecular surface.

In addition, VAE may be used to project MEMS into a latent space. Unlike GPLVM, which is a nonparametric method, VAE relies on neutral networks (and associated trainable parameters) to reduce dimensionality. VAE utilizes a multivariate Gaussian function to regularize the latent data structure to approximate the latent space (via Variation Interference). The encoder may be regarded as projecting the input data onto a latent Gaussian manifold, whose mean and covariance functions are trained by adjusting the neural network parameters via the decoder. In practice, each dimension of the Gaussian distribution is considered independent (i.e., the off-diagonal elements of the covariance matrix are treated as zero).

Both methods offer their own advantages and disadvantages. GPLVM provides more expression because of various choices of kernel functions and consideration of the full covariance matrix. It is, however, computationally expensive, especially for processing a large amount of data (mainly due to the inversion of the covariance matrix). Sparse GP may help but relies on the posterior approximation by variational inference. Importantly, GPLVM works directly on the (unknown) latent variables and has little or no capability to extract features from “raw” data. Conversely, VAE utilizes neutral networks and various architectures (e.g., CNN) to extract and project features to a latent space. On the other hand, with no correlation typically considered between latent variables, VAE may lead to ambiguities in recovering an input; it also suffers from the latent variables not being able to encode the input information (so-called Posterior Collapse75). Comparing using VAE to encode MEMS directly as the image format, the recovered images were fuzzy—a typical observation in deep learning, especially when the number of training data is small or moderate. Nonetheless, given the prowess of neural networks in extracting features from MEMS, VAE is a worthy alternative to construct the latent space of molecules.

Projecting MEMS of molecules into a low-dimensional latent space may potentially overcome COD. Because the smooth Gaussian functions are used to approximate the posterior, using GPLVM or VAE to conduct the dimensionality reduction can further make it effective to sample the latent space. As illustrated in FIG. 21, a functional surface may be explored in the latent space between the latent variables (X) and the molecular property of interest (Y) that is “tagged” to each data point (i.e., molecule). The (unknown) functional surface may be approximated by a GP (as surrogation function), and finding an optimal value by BO follows the Bayes' theorem. Briefly, BO is an iterative optimization method, and at the initial step, the GP posterior is established by the available latent variables projected from MEMS feature matrices. Then reiteratively, at each step, GP regression is conducted over the whole sample space (within bounds), and the best sampling point is identified and further used to update the current posterior. Finding the best sampling point is often done by Expected Improvement (as acquisition function), which compares the predicted means of all sampling points to the current best mean (of the molecular property of interest) and pick the best new point. Because GP regression is done analytically, finding the maximal point may be achieved by optimizing the acquisition function, augmented as probability distribution functions, to enable an “exploration” space around each sampling point. The reiteration stops after the best value is converged. Therefore, it is likely that BO may lead to the finding of the global maximum.

With a given MEMS matrix, the deep learning model shown in FIG. 21 is aimed to generate a weighted adjacency matrix of the atoms in a molecule. The adjacency matrix represents the graph of chemical bonds with the weights signifying the types of the bonds. Additionally, the weight of a bond to denote the two atoms that form the bond may be used. For example, 11 may indicate a single bond between C—H and 12 is O—H single bond. Similar definitions may be well developed in a popular force field. Alternatively, two concurrent deep learning models may be trained to respectively output a regular adjacency matrix and a vector of the elements of the molecule. The deep learning architecture will be implemented by Set2Graph. In one embodiment involved in predicting the packing motifs in a supramolecular structure by training a Set2Graph model with a few thousand known crystal structures, ƒ-scores greater than 0.6 were obtained, which may be further improved by using opencut MEMS and more training data. In some embodiments, however, close matching between the molecule that generates its MEMS and the predicted molecule(s) by the MEMS may not occur. A different molecule may yield similar surface electronic properties as those by another molecule. Additionally, because the molecular representation as adjacency matrix is zero-inflated, the stochastic gradient descent used to collectively optimize the training parameters of neural network layers usually fails to lead to high accuracy in predicting the adjacency matrix. Using Graph Embedding (e.g., via SkipGram) and Focal Loss has resulted in obtaining moderate improvements. Fortunately, experimental data is not required as a computed MEMS may be used to train the model (by comparing it with the molecular structure that produces the MEMS). Still, such “imperfection” may be highly beneficial for molecular design, as the model may allow some degree of hallucination. A final step of valence check may be included to validate a generated structure.

While MEMS may be transformative in chemical learning, surface embedding is just one of many ways to utilize a molecule's quantum chemical constitution to dimensionally reduce the representation of the molecules. Alternative embeddings such as an iso-surface of electron density, the Fiedler vector, projecting surface manifold on the Fiedler vector (1D), or GP directly applied to electronic quantities on a molecular surface may be used in such alternative embodiments. To complement deep learning of ligands, MEMS of the binding pocket of a protein target may be used to screen and find appropriate molecules. Further alternatives involve establishing connections by deep learning between MEMS and the Gaussian atomic orbitals of the underlying molecule. Also, there is the opposite side of COD-Blessing of Dimensionality—where “generic high-dimensional datasets exhibit fairly simple geometric properties,” when MEMS is applied in chemical learning, wherein after an initial processing of MEMS for discovery, prediction and de novo development, further dimensionality may be added back into a particular data set for optimization.

Further embodiments involve MEMS kernels which are more expressive than shape-context matrices in recovering the chemical information of the “underlying” molecule, particularly relevant for solubility prediction. Moreover, by directly kernelize the electronic attributes on a molecular surface without going through the embedding process one may avoids information loss due to the embedding. These exemplary approaches may be referred to as Manifold Kernelization of Molecular Surface (MKMS). Manifold kernels in many situations improve the accurate and salient knowledge in retaining the electronic information of a molecule, especially in generative deep learning for de novo design of molecules. While other types of molecular surfaces are processed similarly, Hirshfeld surfaces are illustrated in this disclosure. A Hirshfeld surface generated by methods found in the Tonto reference (Jayatilaka, D.; Grimwood, D. J., Tonto: A Fortran based object-oriented system for quantum chemistry and crystallography. In Computational Science-ICCS 2003, Pt Iv, Proceedings, Sloot, P. M. A.; Abramson, D.; Bogdanov, A. V.; Dongarra, J. J.; Zomaya, A. Y.; Gorbachev, Y. E., Eds. 2003; Vol. 2660, pp 142-151) in combination with vertices being further optimized by isotropic meshing using the methods of the Cignoni reference (Cignoni, P.; Callieri, M.; Corsini, M.; Dellepiane, M.; Ganovelli, F.; Ranzuglia, G. In MeshLab: an Open-Source Mesh Processing Tool, Sixth Eurographics Italian Chapter Conference, 2008; pp 129-136) are used in one embodiment of the invention. With mesh vertices being input, in this exemplary embodiment, to a C++ program to produce 2-D points of MEMS. To generate an embedding a method of Neighbor Retrieval Visualizer was used (for example, that disclosed in Venna, J.; Peltonen, J.; Nybo, K.; Aidos, H.; Kaski, S., Information retrieval perspective to nonlinear dimensionality reduction for data visualization. Journal of Machine Learning Research 2010, 11, 451-490). This specific process optimizes the distances among embedding points to preserve the local neighborhood of surface vertices. Specifically, it is evaluated as the probability of vertex j in the neighborhood of vertex i:

$${p}_{j \vert i} = \frac{exp ( - {g}_{i j}^{2} / {σ}_{i}^{2} )}{{\sum }_{k \neq i} exp ( - {g}_{i k}^{2} / {σ}_{i}^{2} )} \qquad ( eq . 8 )$$

where gᵢⱼ is the geodesic distance and σ is a parameter to determine the neighborhood coverage for i. A similar probability is defined by the Euclidean distance between the points i and j on the lower-dimensional embedding:

$${q}_{j \vert i} = \frac{exp ( - {d}_{i j}^{2} / {σ}_{i}^{2} )}{{\sum }_{k \neq i} exp ( - {d}_{i k}^{2} / {σ}_{i}^{2} )} \qquad ( eq . 9 )$$

The cost functions consists of two weighted Kullback-Leibler (KL) divergences between the two probability distributions in order to balance false positive and negatives:

$$λ {\sum }_{i = 1}^{N} {\sum }_{{j = 1}_{j \neq i}}^{N} {p}_{j \vert i} log \frac{{p}_{j \vert i}}{{q}_{j \vert i}} + ( 1 - λ ) {\sum }_{i = 1}^{N} {\sum }_{{j = 1}_{j \neq i}}^{N} {q}_{j \vert i} log \frac{{q}_{j \vert i}}{{p}_{j \vert i}} \qquad ( eq . 10 )$$

The parameter, λ, is to weight the two KL divergences; the inventors of the present invention found a value of 0.95 works well in embodiments of the invention. In addition, σᵢ is dynamically adjusted based on the input data (i.e., surface vertices) and the data density around each point, and compared to a “perplexity” hyperparameter, which was identified to be 30 in a particular embodiment.

Electronic properties on the molecule surface are then pointwisely transformed to the MEMS. The properties of single molecules, including electrostatic potential (ESP), nucleophilic Fukui function (ƒ⁺), electrophilic Fukui function (ƒ⁻), and dual descriptor of Fukui function (ƒ²), are calculated by the known Gaussian 09 method (Gaussian, Inc., Wallingford CT). For several exemplary embodiments disclosed herein, including those molecules in the solubility prediction, their conformations were respectively extracted from their crystal structures and partially optimized only for the hydrogen atoms.

To featurize MEMS for deep learning, including Shape-context Featurization of MEMS, a numerical method enhancing a method disclosed in the Belongie reference was developed (Belongie, S.; Mori, G.; Malik, J., Matching with shape contexts. Statistics and Analysis of Shapes 2006, 81-105). Show in FIG. 12, a feature matrix consists of rows of key points, which are the closest surface vertices to the respective atoms of the molecule in 3-D (denoted by atom indices on the figure). The intensities surrounding a key point on a MEMS image are spatially separated in predetermined bins along the radial direction. Each radial bin may be further divided into angular bins, where the angular direction is calculated against the geometric center to allow the rotational invariance of the feature matrix. When used in deep learning, it is the original values of the respective electronic properties that are processed. In one exemplary embodiment, each row in the feature matrices comprises 16 radial bins, each of which has 4 angular bins; on the other hand, there are 32 radial bins in FIG. 12.

In another exemplary embodiment, a deep-learning effort utilized selected 123 molecules from a curated dataset from the first and second solubility challenge tests similar to predictive models in two Llinas references (Llinas, A.; Glen, R. C.; Goodman, J. M., Solubility challenge: Can you predict solubilities of 32 molecules using a database of 100 reliable measurements? Journal of Chemical Information and Modeling 2008, 48 (7), 1289-1303; and Llinas, A.; Avdeef, A., Solubility Challenge Revisited after Ten Years, with Multilab Shake-Flask Data, Using Tight (SD similar to 0.17 log) and Loose (SD similar to 0.62 log) Test Sets. Journal of Chemical Information and Modeling 2019, 59 (6), 3036-3040), being randomly split into 9:1 as the training and testing sets. Selection of the molecules was limited to those with one molecule in the asymmetric unit (i.e., Z′=1). This exemplary embodiment involves Hirshfeld surfaces of the crystal structures of these molecules being calculated, and further dimensionality-reduced to manifold embeddings. Respective electronic properties (electron density, ESP, and Fukui functions) are then evaluated of the single molecules with the conformations extracted from respective crystals. Feature matrices are then derived by the shape context approach and used as the input for deep learning. The input of each molecule consisted of several feature matrices. DeepSets was adapted as the architecture of deep learning as proposed by the Zaheer reference (Zaheer, M.; Kottur, S.; Ravanbhakhsh, S.; Poczos, B.; Salakhutdinov, R.; Smola, A. J. In Deep Sets, 31st Annual Conference on Neural Information Processing Systems (NIPS), Long Beach, CA, December 4-9; Long Beach, CA, 2017); self-attention then used as the sum-decomposition as demonstrated in Set2Graph environment as disclosed in the Vaswani reference (Vaswani, A.; Shazeer, N.; Parmar, N.; Uszkoreit, J.; Jones, L.; Gomez, A. N.; Kaiser, L.; Polosukhin, I. In Attention Is All You Need, 31st Annual Conference on Neural Information Processing Systems (NIPS), Long Beach, CA, December 4-9; Long Beach, CA, 2017) then modified to consider intermolecular direct contacts.

In an alternative embodiment, the attention architecture may be described as follows:

$$\sum ( X ) = softmax ( \frac{tanh ( {f}_{1} ( X ) ) \cdot {{f}_{2} ( X )}^{T}}{{d}^{1 / 2}} \cdot A ) \cdot X \qquad ( eq . 11 )$$

where X is the input set of MEMS features, d is the feature dimension of X divided by a predetermined number (typically 10), and ƒ₁ and ƒ₂ are the query and key functions of self-attention, which are implemented by MLP or multilayer perceptron. A consists of adjacency matrices of the molecules, which donate the close contacts between the atoms of adjacency molecules in crystal. Notably, the self-attention mechanism is permutation invariant and is widely used to capture the intra-correlations of the input features. Additionally, regularization of each DeepSets is done by batch normalization (BN) and Leaky ReLU; weight decay and dropout (typically set at 50%) are also considered in the PyTorch optimizer to further mitigate model overfitting. When five 4×16 shape-context matrices were used for each molecule (including electron density, positive ESP, negative ESP, ƒ⁺, and ƒ⁻), there were 320 dimensions of each key point (or atom). In one exemplary embodiment of the invention, 12 DeepSets layers of (512, 512, 256, 256, 64, 64, 32, 32, 16, 16, 4, 4) feature dimensions are used, with Learning rate being set at 0.0001, L1 loss chosen as the cost function, all optimized by the Adam algorithm in PyTorch.

Embodiments of the invention provide modeling for molecules that more accurately predict chemical behavior. When molecules (in a dataset) may be differentiated, any descriptor may work even in a ML/DL model. Yet, if such a descriptor carries little chemical intuition or information, training with the descriptors is most likely to be difficult, requiring sophisticated models (and chemical rules), as well as a large amount of data to approximate the underlying one-to-one relationship between the descriptor and the property of interest. Conversely, when a molecular representation not only differentiates molecules but also bears rich chemical information such as those used in various embodiments of the invention, the training is straightforward even with a small set of data. In addition, the inventors are considerably expanded their studies by utilizing electron-density iso-surfaces of single molecules in the Solubility Challenges, fully optimized or kept of the same conformers when generating Hirshfeld surfaces. In further embodiments, molecules in a much larger dataset, ESOL (1128 molecules), are evaluated which is widely used by benchmarking efforts of machine and deep learning.

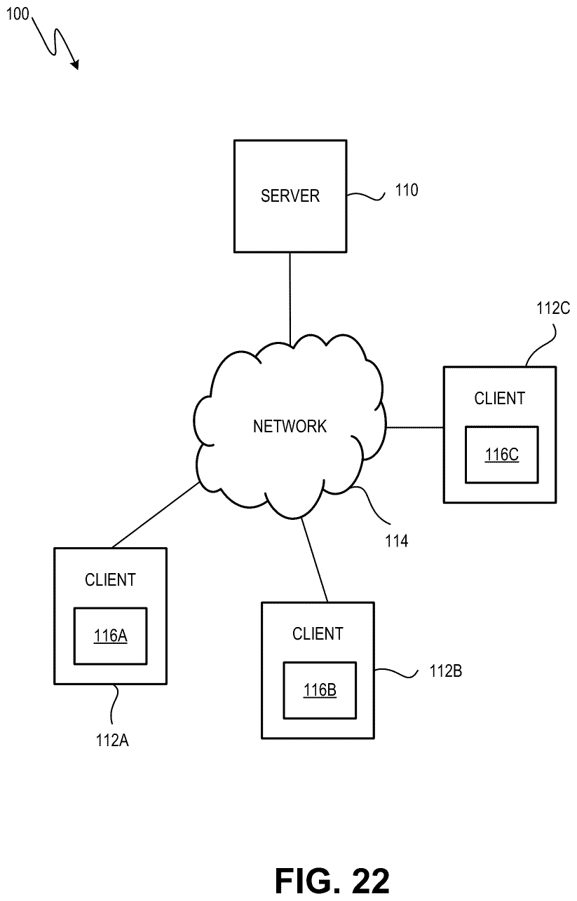

FIG. 22 is a high-level block diagram of a computing environment 100 according to one embodiment, although those skilled in the art of computing recognize that the components of the processing described herein may be conducted on a single computing machine or distributed over wired or wireless networks. FIG. 22 illustrates server 110 and three clients 112 connected by network 114. Only three clients 112 are shown in FIG. 22 in order to simplify and clarify the description. Embodiments of the computing environment 100 may have thousands or millions of clients 112 connected to network 114, for example the Internet. Users (not shown) may operate software 116 on one of clients 112 to both send and receive messages network 114 via server 110 and its associated communications equipment and software (not shown).

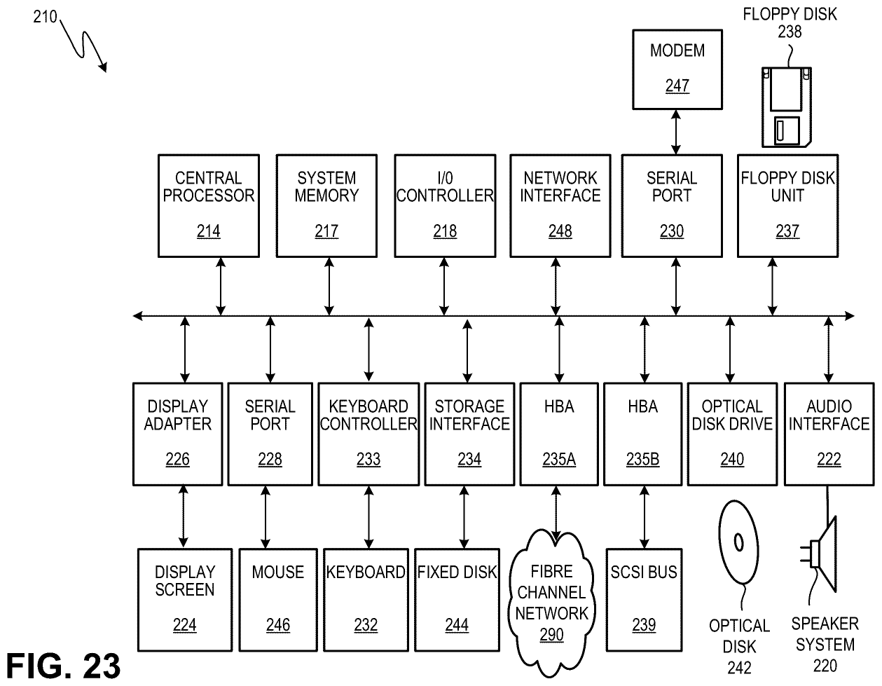

FIG. 23 depicts a block diagram of computer system 210 suitable for implementing server 110 or client 112. Computer system 210 includes bus 212 which interconnects major subsystems of computer system 210, such as central processor 214, system memory 217 (typically RAM, but which may also include ROM, flash RAM, or the like), input/output controller 218, external audio device, such as speaker system 220 via audio output interface 222, external device, such as display screen 224 via display adapter 226, serial ports 228 and 230, keyboard 232 (interfaced with keyboard controller 233), storage interface 234, disk drive 237 operative to receive floppy disk 238, host bus adapter (HBA) interface card 235A operative to connect with Fibre Channel network 290, host bus adapter (HBA) interface card 235B operative to connect to SCSI bus 239, and optical disk drive 240 operative to receive optical disk 242. Also included are mouse 246 (or other point-and-click device, coupled to bus 212 via serial port 228), modem 247 (coupled to bus 212 via serial port 230), and network interface 248 (coupled directly to bus 212).

Bus 212 allows data communication between central processor 214 and system memory 217, which may include read-only memory (ROM) or flash memory (neither shown), and random-access memory (RAM) (not shown), as previously noted. RAM is generally the main memory into which operating system and application programs are loaded. ROM or flash memory may contain, among other software code, Basic Input-Output system (BIOS) which controls basic hardware operation such as interaction with peripheral components. Applications resident with computer system 210 are generally stored on and accessed via computer readable media, such as hard disk drives (e.g., fixed disk 244), optical drives (e.g., optical drive 240), floppy disk unit 237, or other storage medium (disk drive 237 is used to represent various type of removable memory such as flash drives, memory sticks and the like). Additionally, applications may be in the form of electronic signals modulated in accordance with the application and data communication technology when accessed via network modem 247 or interface 248 or other telecommunications equipment (not shown).

Storage interface 234, as with other storage interfaces of computer system 210, may connect to standard computer readable media for storage and/or retrieval of information, such as fixed disk drive 244. Fixed disk drive 244 may be part of computer system 210 or may be separate and accessed through other interface systems. Modem 247 may provide direct connection to remote servers via telephone link or the Internet via an internet service provider (ISP) (not shown). Network interface 248 may provide direct connection to remote servers via direct network link to the Internet via a POP (point of presence). Network interface 248 may provide such connection using wireless techniques, including digital cellular telephone connection, Cellular Digital Packet Data (CDPD) connection, digital satellite data connection or the like.

Many other devices or subsystems (not shown) may be connected in a similar manner (e.g., document scanners, digital cameras and so on). Conversely, all the devices shown in FIG. 8 need not be present to practice the present disclosure. Devices and subsystems may be interconnected in different ways from that shown in FIG. 8. Operation of a computer system such as that shown in FIG. 8 is readily known in the art and is not discussed in detail in this application. Software source and/or object codes to implement the present disclosure may be stored in computer-readable storage media such as one or more of system memory 217, fixed disk 244, optical disk 242, or floppy disk 238. The operating system provided on computer system 210 may be a variety or version of either MS-DOS® (MS-DOS is a registered trademark of Microsoft Corporation of Redmond, Washington), WINDOWS® (WINDOWS is a registered trademark of Microsoft Corporation of Redmond, Washington), OS/2® (OS/2 is a registered trademark of International Business Machines Corporation of Armonk, New York), UNIX® (UNIX is a registered trademark of X/Open Company Limited of Reading, United Kingdom), Linux® (Linux is a registered trademark of Linus Torvalds of Portland, Oregon), or other known or developed operating system. In some embodiments, computer system 210 may take the form of a tablet computer, typically in the form of a large display screen operated by touching the screen. In tablet computer alternative embodiments, the operating system may be iOS® (iOS is a registered trademark of Cisco Systems, Inc. of San Jose, California, used under license by Apple Corporation of Cupertino, California), Android® (Android is a trademark of Google Inc. of Mountain View, California), Blackberry® Tablet OS (Blackberry is a registered trademark of Research In Motion of Waterloo, Ontario, Canada), webOS (webOS is a trademark of Hewlett-Packard Development Company, L.P. of Texas), and/or other suitable tablet operating systems.

Moreover, regarding the signals described herein, those skilled in the art recognize that a signal may be directly transmitted from a first block to a second block, or a signal may be modified (e.g., amplified, attenuated, delayed, latched, buffered, inverted, filtered, or otherwise modified) between blocks. Although the signals of the above-described embodiments are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

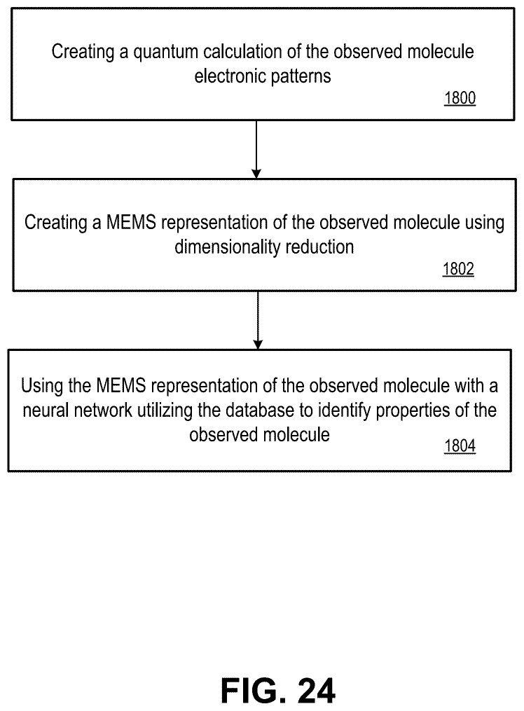

In one embodiment, the present invention relates to the process depicted in the flow chart of FIG. 24.

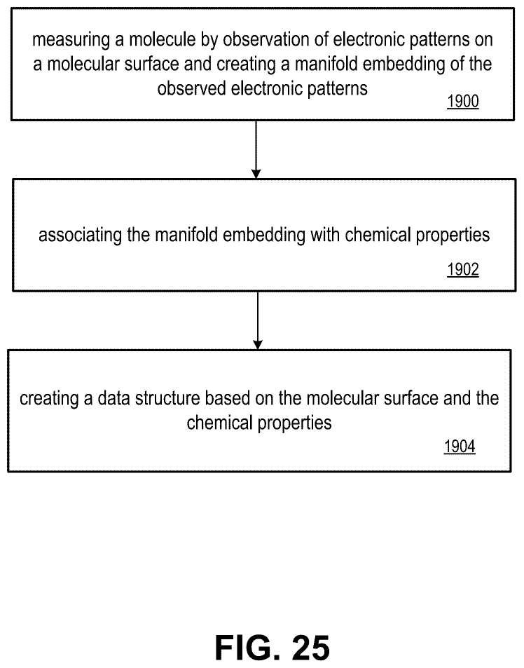

In a second embodiment, the present invention relates to the process depicted in the flow chart of FIG. 25.

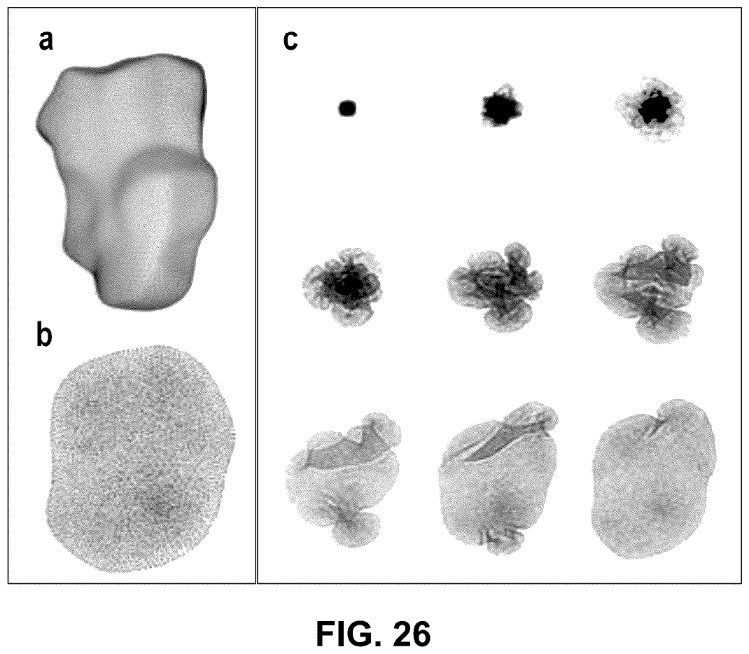

The further development of the present invention involves the dimensionality reduction process of a Hirshfeld surface of tolfenamic acid (metastable or Form II) as is illustrated in FIG. 26. The optimization process is demonstrated in FIG. 20's box c, where each of the series of representational points start with the initially randomized points which are then progressively re-positioned, reaching a local minimum of the cost function by the resultant MEMS. Because a molecular surface is mathematically enclosed, some surface points fall in a wrong neighborhood on the embedding as false positives (distant neighbors in 3-D put in the same neighborhood on MEMS) or false negatives (near neighbors in 3-D separated on MEMS). By developing a basic scheme to estimate the percent of false points, and by defining the neighborhood radius twice of the shortest inter-vertex distance on the 3-D surface, a point is assigned as “outsider” if none of its MEMS neighbors originates from its 3-D neighborhood. From various cases analyzed, the percentage of outsiders was typically 20-40%, depending on the geometry of a molecular surface. Thus, MEMS generated by NeRV seems to retain most of the chemical information on a molecular surface.

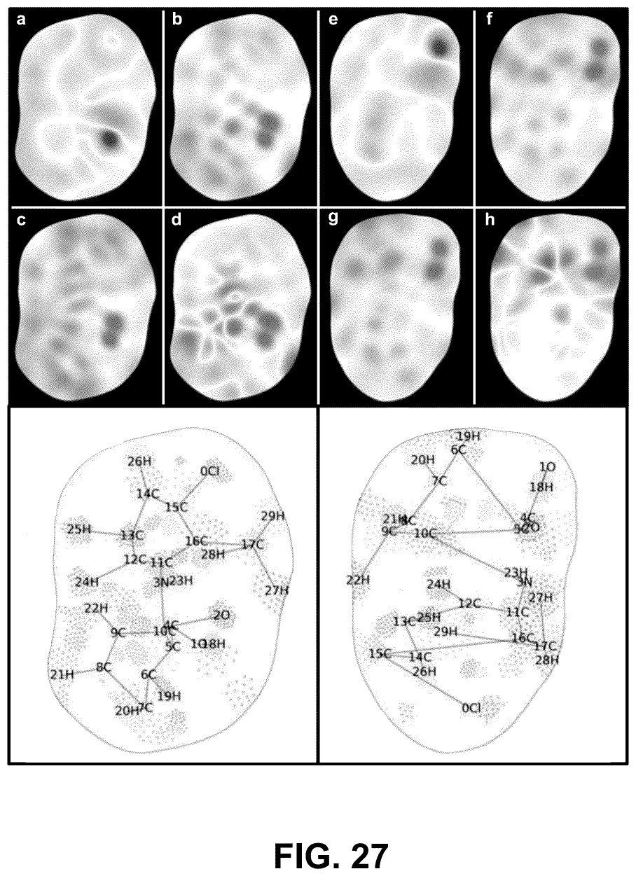

FIG. 27 showcases a few interpolated MEMS that are color-coded with electronic properties on the corresponding manifolds (a-h). The electronic properties (electrostatic potential or ESP, nucleophilic Fukui function or ƒ⁺, electrophilic Fukui function or ƒ⁻, and dual descriptor of Fukui function or ƒ²) were calculated of the single molecules, whose conformations were extracted from the crystal structures. The two MEMS in FIG. 27 are of the same molecule but different conformations, revealing that major electronic properties and spatial patterns are preserved. The subtle and yet significant differences between the two crystal forms are captured by the 2-D embeddings. Note that the color scale in FIG. 27 is relative to the respective electronic attributes. Each image has its largest value scaled to a full byte with positive numbers assigned to the red channel and negative to the blue of the image (opposite for ESP). The most outstanding region of the electronic properties is of the carboxyl group; its adjacent aromatic ring seems more polarized than the other ring.

Interpolation of electronic values on the MEMS in FIG. 8 was conducted by Gaussian-based radial basis functions (RBFs). Because of Gaussian kernels, the interpolation preserves dominant electronic attributes but smooths out minor features on the embeddings. In the present case, most false positives and negatives were averaged out by the interpolation process. FIG. 9 demonstrates one extreme case where 3247 points were retained out of 9011 total embedding points, while the RBF interpolation still led to the preservation of major electronic patterns. No false points were removed before conducting RBF interpolation of electronic features in the present studies. Importantly, the interpolation suggests that the underlying dimensionality of electronic attributes on MEMS is much smaller than that of MEMS itself (as a matrix or RGB image) and likely at the same order of the number of atoms. This is not intuitively surprising, as electronic features on a molecular surface spread over domains comparable with the size of an atom.

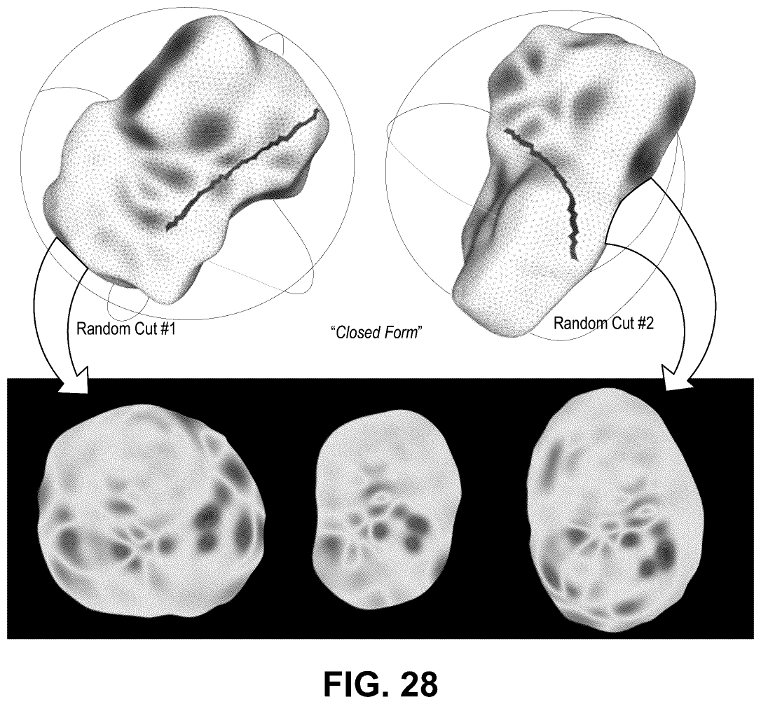

Nonetheless, to minimize the information loss due to false positives and negatives, an attempt was made to cut a molecular surface by removing the connectivity between surface vertices along the geodesic between two vertices on the surface. The vertices along the cutting line are forced to become the boundary points on the embedding. FIG. 28 shows MEMS of the same Hirshfeld surface (F2) of tolfenamic acid that is randomly cut; both cut MEMS have no embedding point in a wrong neighborhood (with respect to the cut surfaces). In comparison, the MEMS of the uncut surface (“closed”) has about 40% false points. Nonetheless, false negativity of the points along the cutting line is entailed. It is thus posited that two or more cut MEMS be combined to mitigate the ambiguity and truthfully represent a molecule. Note that for the molecules in the solubility prediction, cutting was done not randomly but between two surface points intercepted by principal axes of mesh points. The matrix or image format of MEMS may be directly utilized in a machine learning model, e.g., by convolutional NNs (CNNs). However, the true dimensionality of a MEMS is much smaller than that of the embedding itself, as implied by the Figures. While MEMS is translation- and rotation-invariant, its orientation when placed on 2-D is random but should be orientation-invariant. It became desirable to further featurize and reduce the dimensionality of MEMS, among which the shape context used in computer vision was adopted and discussed herein.

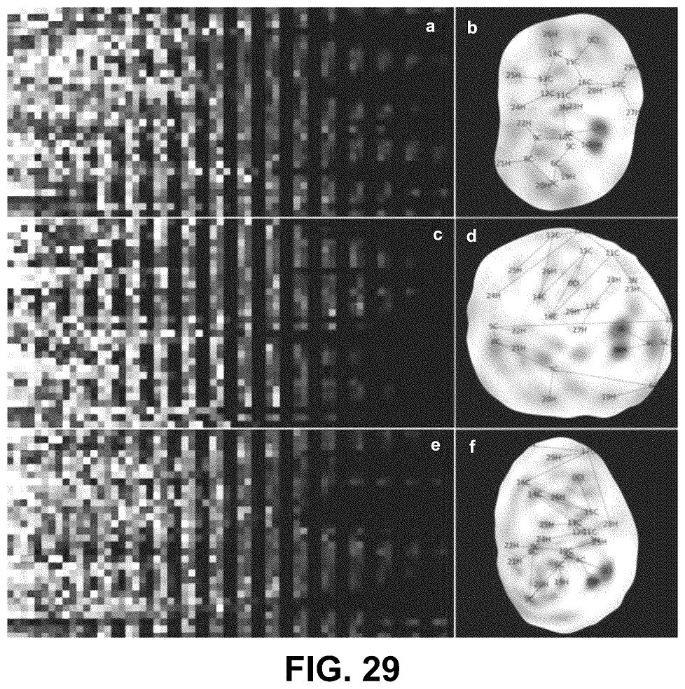

Shown in FIG. 29, a shape-context matrix consists of rows of key points, which are chosen as the closest surface vertices to the respective atoms of the molecule. In FIG. 23, shape-context matrices of MEMS (ƒ⁻) derived from the same surface manifold: closed (23b) and randomly cut (23d and 23f). Key points are marked on the MEMS. The color scheme of the matrix plots varies from blue to green and to red as bin value increases. When used in calculation, the absolute values of the respective electronic properties of the bins are processed. The three MEMS (F⁻) and corresponding shape-context matrices in FIG. 29 are derived from the same molecular surface (FIG. 28; with a different electronic property). Despite the MEMS derived differently, their shape-context matrices appear highly similar. The similarity distances calculated by the Earth Mover's Distance (EMD) algorithm are 1.44 (between FIGS. 23a and 23c), 1.18 (between 23a and 23e), and 1.35 (between 23c and 23e). In comparison, the EMD between FIGS. 21c and 21g is 1.70. The similarity suggests that shape context saliently captures the spatial distribution of electronic attributes on a molecular surface.

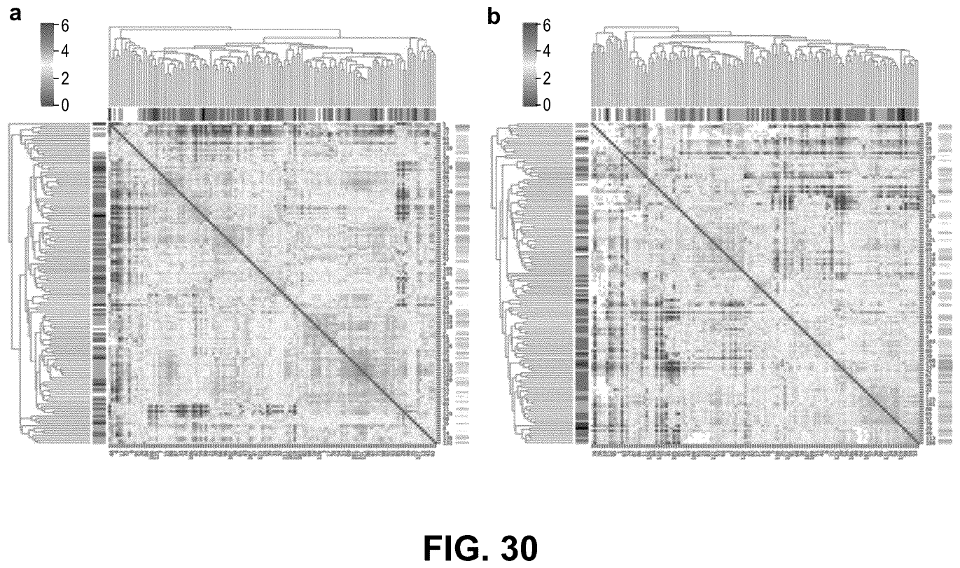

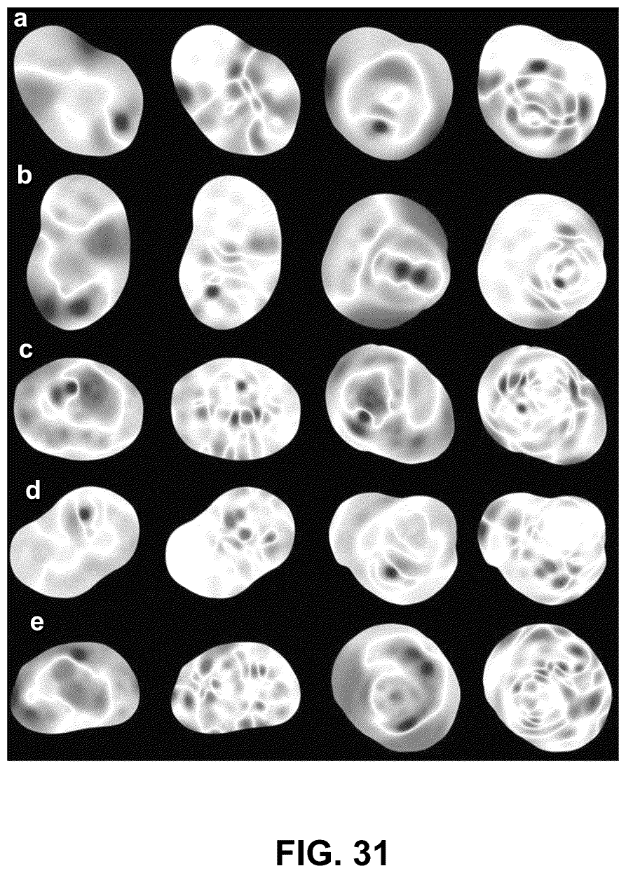

To further examine manifold cutting on shape-context featurization, similarities between the feature matrices of Hirshfeld surfaces of the 133 molecules used in the solubility prediction were calculated. FIG. 30 shows two comparing heatmaps of EMD values along with clustering dendrograms of the similarities. Heatmap and cluster analysis of EMD values between shape-context matrices of the negative ESP on closed [FIG. 24 (a)] or cut MEMS [FIG. 30b)] are calculated of Hirshfeld surfaces of 133 molecules utilized in the deep-learning prediction of water solubility. Evenly indexed molecules are marked on the right and oddly indexed ones are marked on the bottom. The indices can be found in Table 3. The gray bar under each dendrogram is of solubility with white, light gray, mid-gray, dark gray, and black marking Log S>−2.0, >−4.0 and <=−2.0, >−6.0 and <=−4.0, >−8.0 and <=−6.0, and <−8.0, respectively. The color bar on the far right is of the averaged EMD of each molecule among close and its four cut MEMS, corresponding to the respective molecules by the rows.

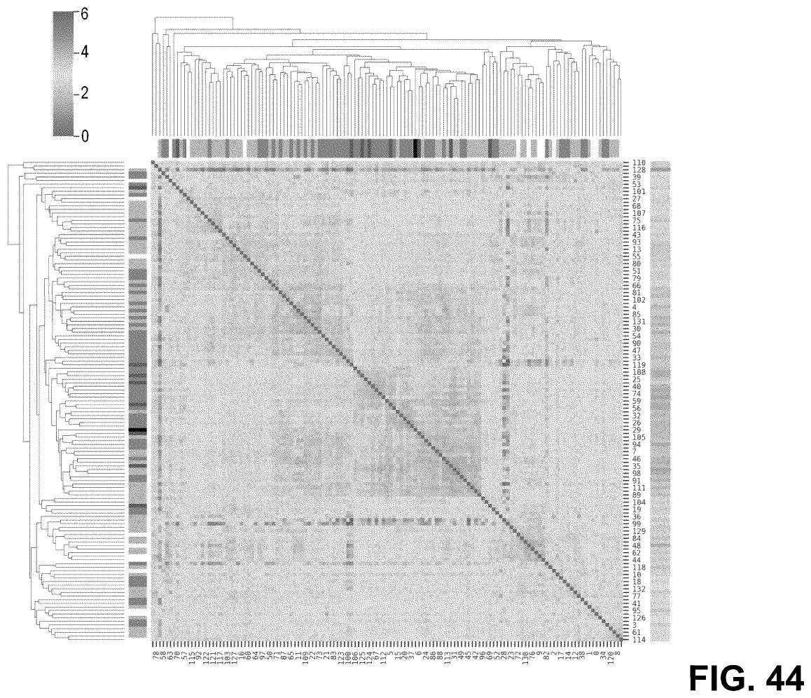

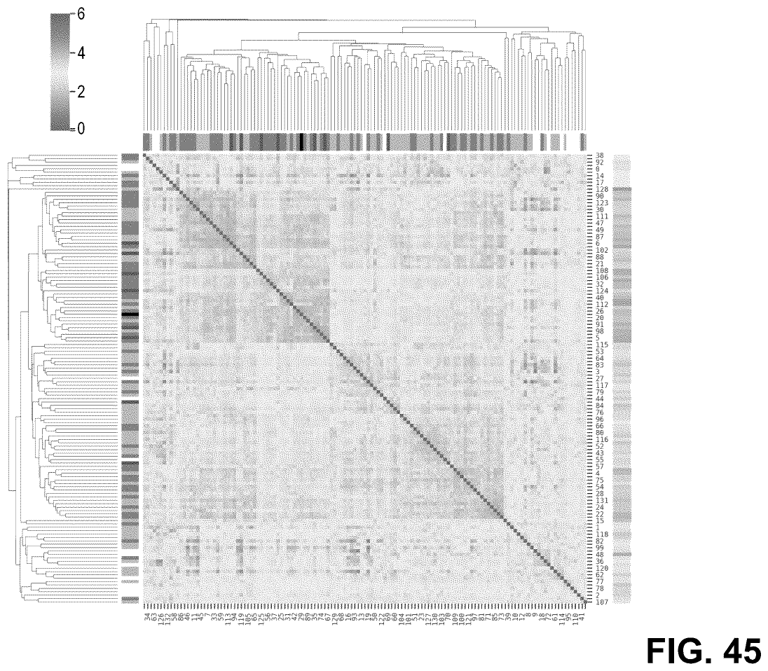

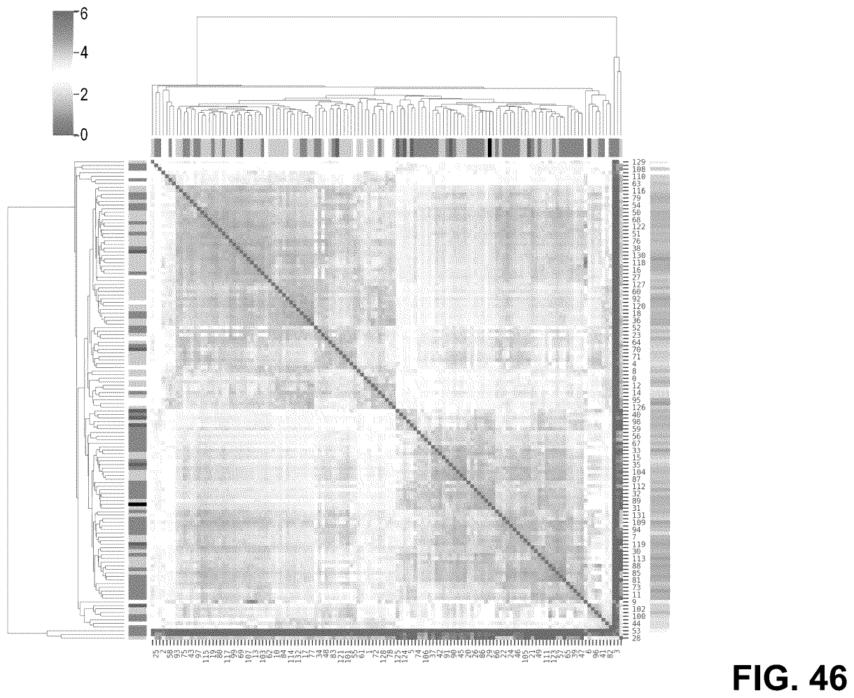

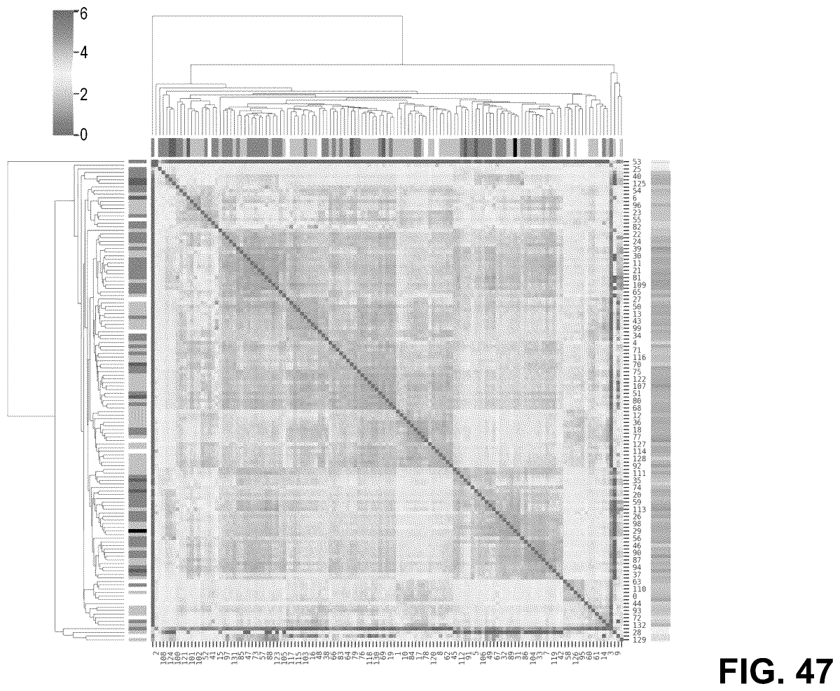

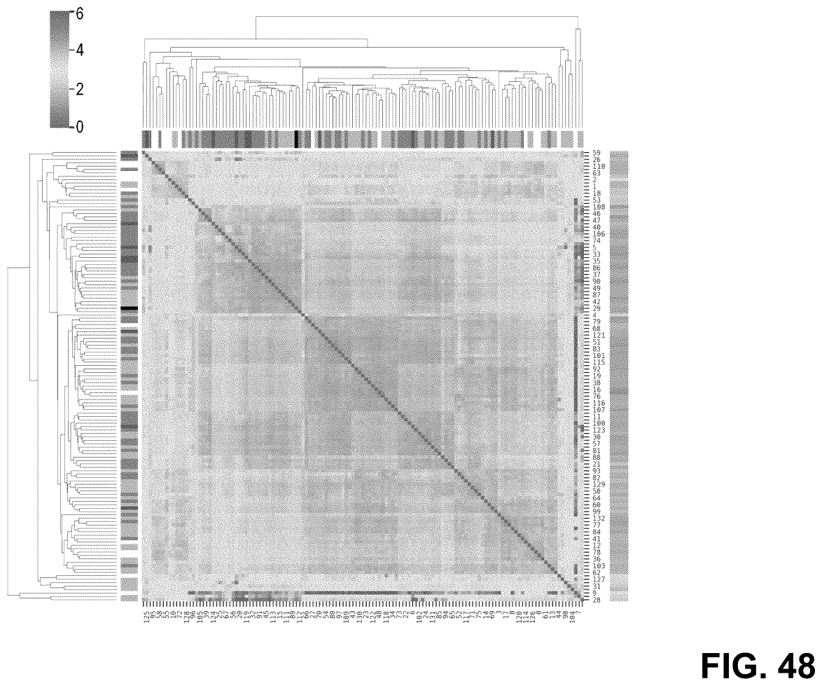

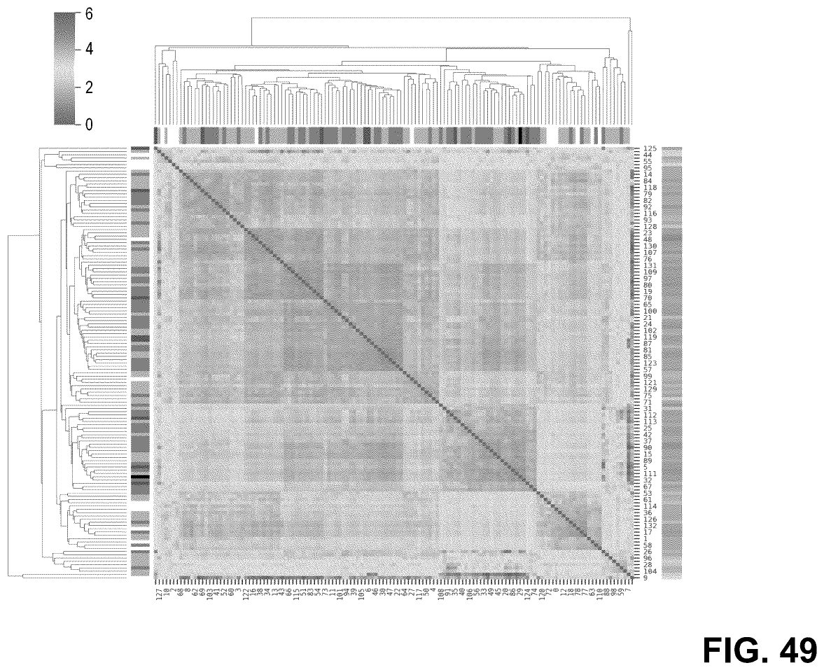

FIGS. 44-49 provide more detailed heatmap analysis. FIG. 44 shows heatmap and cluster analysis of EMD values between shape-context matrices of the positive electrostatic potentials on closed MEMS calculated of Hirshfeld surfaces of 133 molecules utilized in the deep-learning prediction of water solubility. Evenly indexed molecules are marked on the right and oddly indexed ones are marked on the bottom. The indices may be found in Table 3 below. The gray bar under each dendrogram is of solubility with white, light gray, mid-gray, dark gray, and black marking Log S>−2.0, >−4.0 and <=−2.0, >−6.0 and <=−4.0, >−8.0 and <=−6.0, and <−8.0, respectively. The color bar on the far right is of the averaged EMD of each molecule among close and its four cut MEMS, corresponding to the respective molecules by the rows. FIG. 45 shows heatmap and cluster analysis of EMD values between shape-context matrices of the positive electrostatic potentials on cut MEMS calculated of Hirshfeld surfaces of 133 molecules utilized in the deep-learning prediction of water solubility, similar to FIG. 44. FIG. 46 shows heatmap and cluster analysis of EMD values between shape-context matrices of the nucleophilic Fukui functions on close MEMS calculated of Hirshfeld surfaces of 133 molecules utilized in the deep-learning prediction of water solubility, similar to FIG. 44. FIG. 47 shows heatmap and cluster analysis of EMD values between shape-context matrices of the nucleophilic Fukui functions on cut MEMS calculated of Hirshfeld surfaces of 133 molecules utilized in the deep-learning prediction of water solubility, marked as FIG. 44. FIG. 48 shows heatmap and cluster analysis of EMD values between shape-context matrices of the electrophilic Fukui functions on close MEMS calculated of Hirshfeld surfaces of 133 molecules utilized in the deep-learning prediction of water solubility, denoted as FIG. 44. FIG. 49 also shows heatmap and cluster analysis of EMD values between shape-context matrices of the electrophilic Fukui functions on cut MEMS calculated of Hirshfeld surfaces of 133 molecules utilized in the deep-learning prediction of water solubility, in keeping with FIG. 44.

Several observations may be made by perusing the similarity maps. The averaged EMD values between the closed and four cuts of the same molecules are generally smaller than the values between molecules. The two heatmaps share similar patterns in general, suggesting that manifold cutting does not alter the overall differences among the molecules or introduce significant falsehood (i.e., false negatives on MEMS). Similarity values of Fukui functions are smaller than those of ESP, indicating that MEMS of Fukui functions are less dissimilar. However, given the much localized and richer patterns of Fukui functions as compared to ESP, the setup to calculate EMD with 4 angular bins (and 12 radial ones) might not be discerning enough for Fukui functions, warranting further studies. As the molecules are largely differentiated and consistently distributed on the maps, the featurization scheme by shape context seems capable of retaining the electronic properties on the molecular surfaces. Interestingly, the clustering apparently bears no correlation with the respective solubility values. This may not be surprising as the EMD or similarity values can be regarded as low-dimensional (non-linear) projections of the MEMS. The full shape-context features need to be considered to predict the solubility.

Solubility is one of the essential physicochemical properties of molecules. Being a grand challenge in chemistry, predicting a molecule's solubility has been attempted in many studies, ranging from empirical and data-driven models to thermodynamic evaluations and to computer simulations. Solubility is a property of solid state, determined by intermolecular interactions among the solute and solvent. Two solubility challenges were recently held with enthusiastic participations. Various degrees of performance were achieved, but the space to improve still remains widely open. Considering experimental errors in obtaining solubility data, one log unit between experimental and predictive values has been a widely-regarded bar of evaluation. Still, larger experimental errors and inter-laboratory variabilities are expected, compounding the difficulties in solubility prediction. Taking advantage of the four well-curated datasets from the two challenge calls, a deep-learning framework was developed to test the applicability of MEMS and shape-context matrices for solubility prediction. As solubility is a property of crystal, the initial analysis was based on the manifold embeddings that were calculated of the drug crystals (i.e., Hirshfeld surfaces). Crystal structures of 133 molecules were obtained from the datasets and used in deep learning. Several representative MEMS are shown in FIG. 31.

The calculated MEMS (ESP and F²) shown in FIG. 31 corroborate that the embeddings preserve the essential electronic information, both spatial distributions and numerical scales, on a particular molecular surface. FIG. 31 shows ESP and ƒ² MEMS of four selected molecules used in the deep-learning prediction, acetaminophen 25(a), benzocaine 25(b), carbamazepine 25(c), flufenamic acid 25(d), and sulfisoxazole 25(e). Of each molecule, the first two are of closed (ESP and ƒ²) and the other two of cut MEMS. The color scheme is the same as that in earlier Figures. Compared with the closed counterparts, the cut MEMS retain extra details of electronic values from a molecular surface but still share the major graphical features. The red spots on the ESP MEMS mark electron-concentrated and the blue indicate electron-deprived regions. Additionally, the red spots on the ƒ² MEMS are electron-hungry and the blue electron-donating. A local region with larger ESP or ƒ², either red or blue, is associated with greater contributions to intermolecular interactions, including hydrogen bonding and aromatic stacking. A hotspot of ESP is typically echoed by larger values of Fukui functions, which are more localized and featured. The electronic attributes on a molecular surface and thus MEMS not only determine the strength of interacting with another molecule, but the local, spatial variations also control the locality of molecular interactions. From the machine-learning perspective, the distinctive graphical patterns of the MEMS, especially with combination of both ESP and Fukui functions, can greatly enhance the discerning power to represent different molecules.

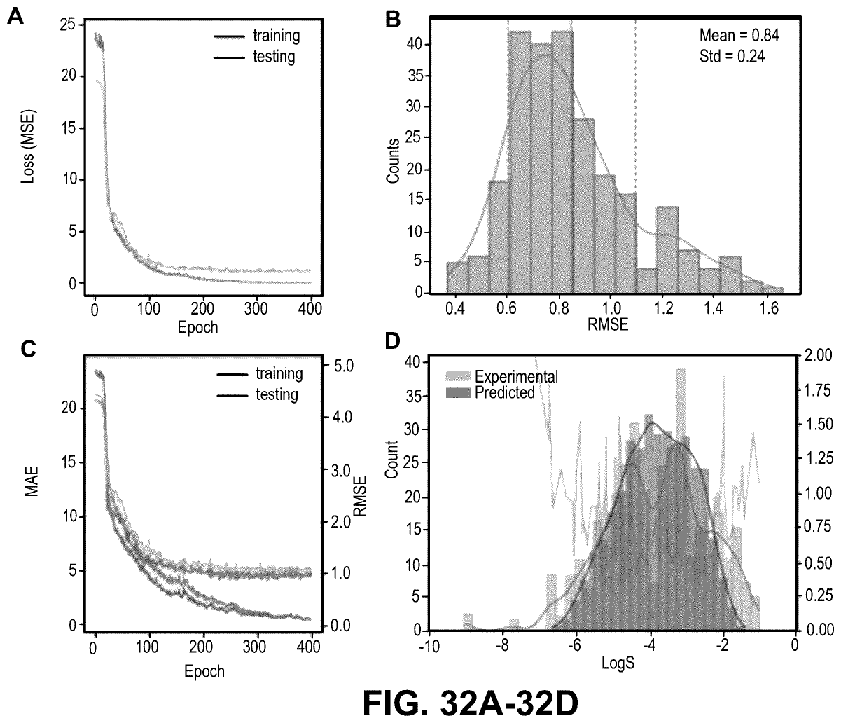

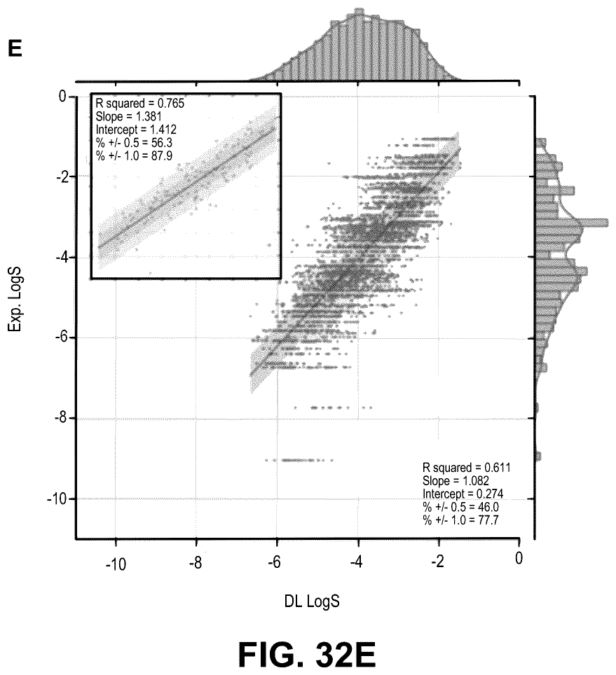

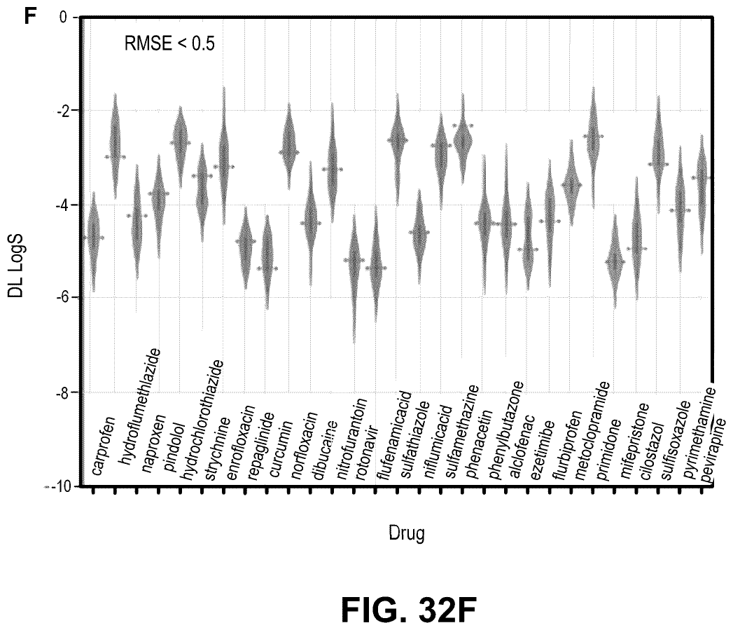

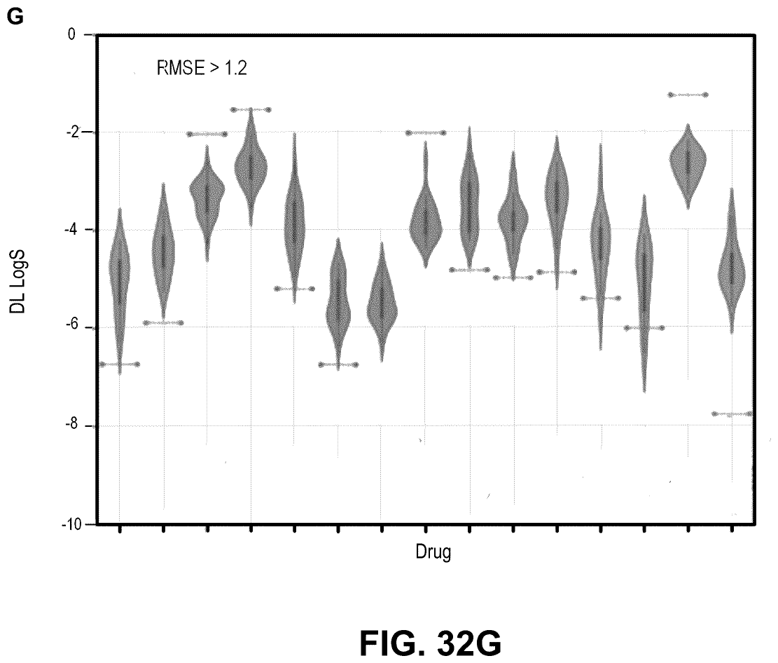

With shape-context matrices of four cut MEMS of each molecule, one set of deep-learning results by 9:1 splitting of the 133 molecules as training and testing datasets is shown in FIGS. 32A-G, showing prediction results of 256 cross validations (CVs), including prediction MSE (mean-squared error), MAE (mean absolute error), and RMSE (root-mean-squared error) between the predicted and experimental values of a representative learning run (32A and 32B), RMSE distribution of CVs (32C), distributions of predicted and experimental values and RMSE values of each molecule (32D), linear fitting of predicted vs experimental values along with their respective distributions and (32E) and average predicted vs experimental values (32e; inset), best-predicted molecules (RMSE<0.5) with violin plots of predicted values and red dots marking corresponding experimental values (32F), and worst-predicted molecules (RMSE>1.2). For each of 133 molecules, four cut MEMS derived from its Hirshfeld surface were utilized. Solubility is in logarithm unit in this report unless otherwise noted. The training and testing losses of a representative run (32A and 32B) illustrate a typical deep learning process seen among other runs in this study. Out of the 256 CVs, each molecule was predicted at least 16 and at most 44 times. The R-squared value (R²) is 0.61 and about 46.0% and 77.7% of the predicted values fall within half and one logarithm unit from their respective experimental values. Note that when the predicted values from the CVs are averaged of each molecule and plotted against the experimental data (FIG. 32E; inset), R² becomes 0.76; similar trends of large increase in R² can also be seen in other runs in this study by fitting with averaged predicted values. Nevertheless, only fittings by individual predicted data were reported thereafter to better understand the model performance. The distribution of RMSE by the CVs is shown in FIG. 26c with the average of 0.84 between two extremes around 0.4 and 1.6. RMSE of each molecule predicted out of the CVs is shown in FIG. 26d, superimposed with the distributions of the predicted and experimental values (which are also showed in FIG. 32E on the top and right axes, respectively). The results demonstrate that the prediction performance depends on which molecules were included in the testing dataset (and, reciprocally, in the training). As shown in FIG. 26f, the “best” molecules typically have their experimental values between −2.0 and −6.0 where, interestingly, most training data points reside (also shown in FIG. 32E). At the two tails of the data distribution (>−2.0 or <−6.0), fewer experimental points were available; only two molecules have solubility smaller than −7, clofazimine (−9.05) and terfenadine (−7.74). These two molecules were poorly predicted. Among the 15 “worst” molecules, 10 seem to have their solubility values outside or bordering the −2.0 and −6.0 range. It is worth pointing out that there are two datasets in the 2019 Solubility Challenge and the second one has much larger experimental uncertainties (>0.6 as compared to <0.2 of the first dataset). Exemplary trials had 16 molecules taken from the second dataset and 4 of them had poor predictability (FIG. 32G), including clofazimine, terfenadine, chlorprothixene (−5.99), and telmisartan (−6.73), which all reside at the insoluble end of the solubility distribution.

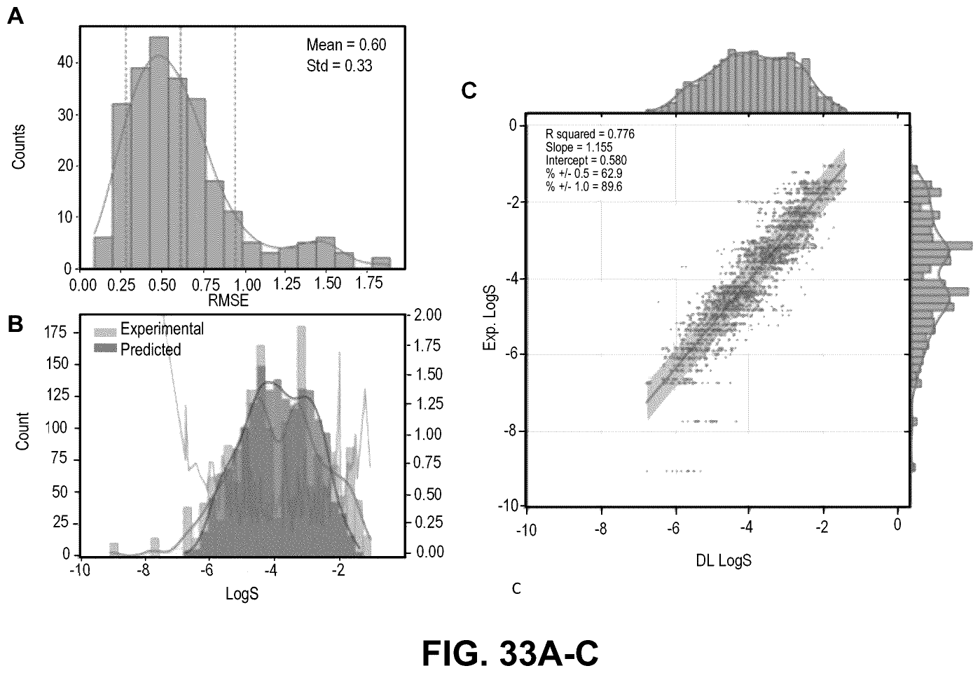

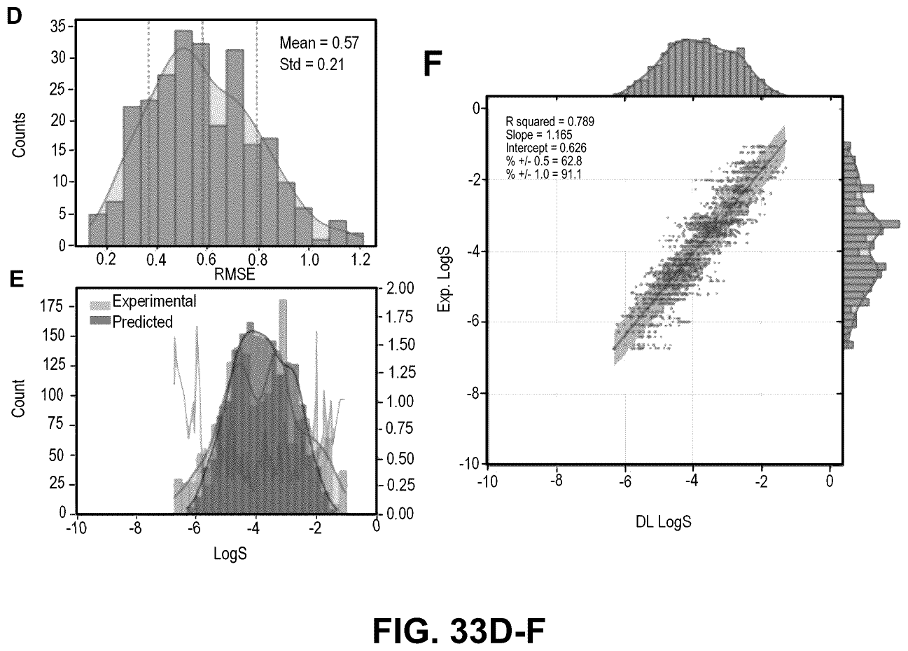

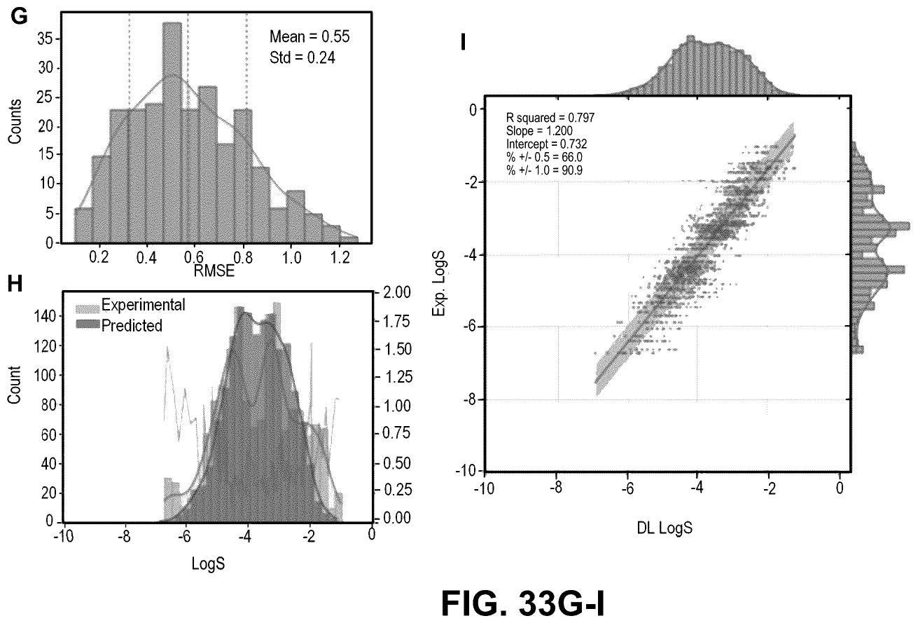

The relatively wide distributions of predictive RMSE values imply the sensitive nature of the deep learning model to the small dataset. At the 9:1 ratio, there were 119 and 14 molecules in the training and testing datasets; the RMSE of 14 randomly assigned molecules in a CV ranges from 0.4 to 1.6 (32C). 95% or 126 of 133 molecules were taken as the training set and conducted deep learning. The numbers of DeepSets layers and features remained the same. The predicted results are highlighted in FIGS. 33A-C. The average RMSE of the CVs decreased from 0.84 to 0.60; R² the linear correlation increased from 0.61 to 0.78 with 62.9% of the molecules within half logarithmic unit. In addition, the RMSE values of the molecules (33B) show similar connections with the distribution of the training data. Smaller values are seen in the middle where more data points are available, while larger values appear at both tails. Again, the two most insoluble molecules, clofazimine and terfenadine, had the worst predicted RMSE (>2.0). As such, another attempt was taken with the same configuration but removed clofazimine and terfenadine from the whole dataset. FIGS. 33D-F show the predicted results, which are clearly improved. The average RMSE from the CVs decreased to 0.57 and R² of the linear correlation increased to 0.79. Furthermore, all 16 molecules that were taken from the second set in the 2019 Solubility Challenge were excluded, which have experimental uncertainties of 0.6 or greater, and utilized 95% of the 117 molecules for deep learning. The results are summarized in FIGS. 27g-i. The averaged RMSE of predicted values from the CVs is improved to 0.55 and R² becomes 0.80 with 66.0% of molecules within half logarithmic unit respectively from their experimental values. Lastly, a test was performed using the 117 molecules as the training and the 16 molecules as the testing dataset. The linear correlation is poor between the predicted and experimental values with R² of 0.04; RMSE of 7 molecules are greater than 1.0 with the average RMSE of the 16 molecules being 1.92. The two most insoluble molecules have the largest prediction errors. The results suggest that prediction performance by deep learning is greatly affected by the distribution of the training data. In the present case, there was no molecule with solubility <−7.0 in the training set. The large experimental uncertainties (>0.6) of the 16 molecules also make the prediction difficult to evaluate.

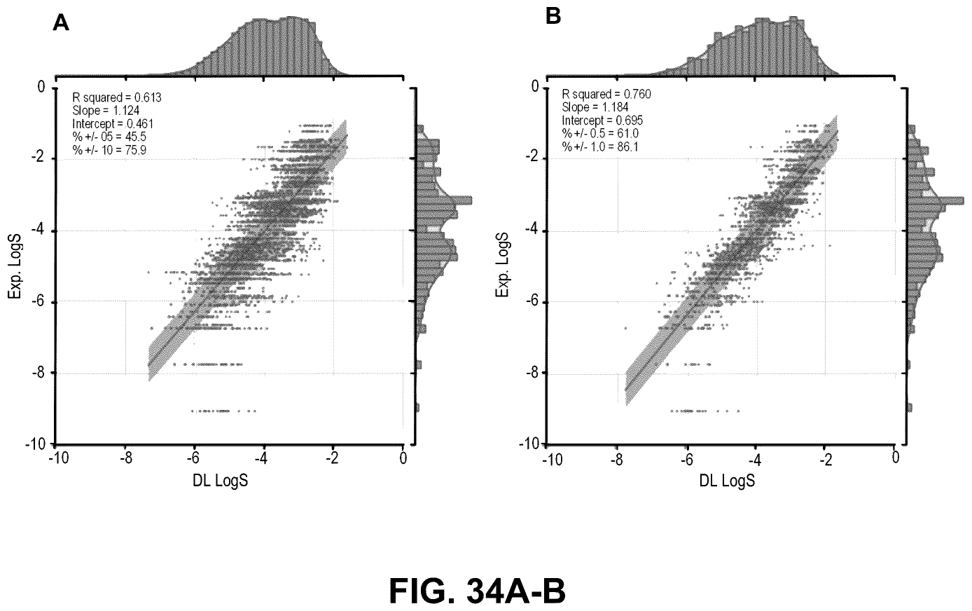

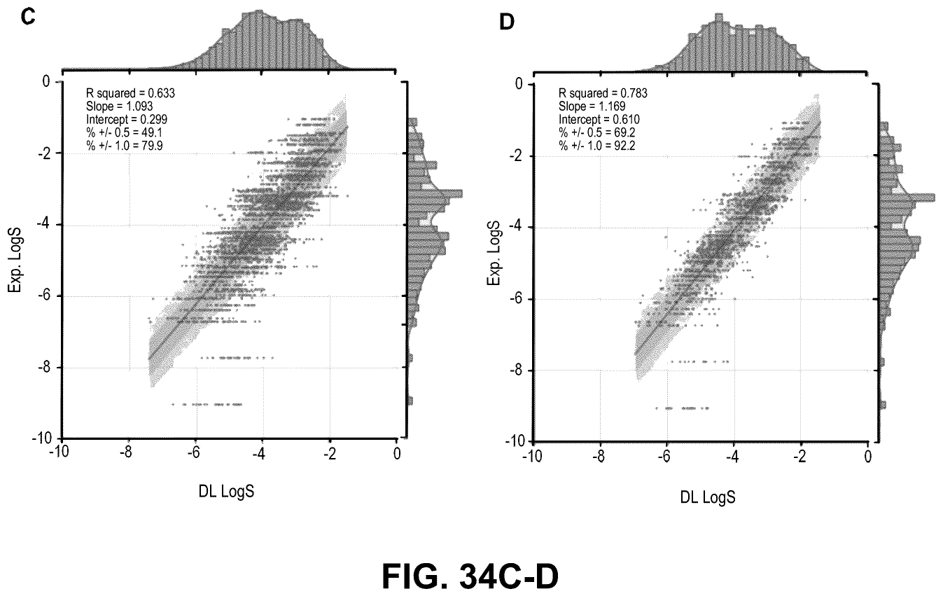

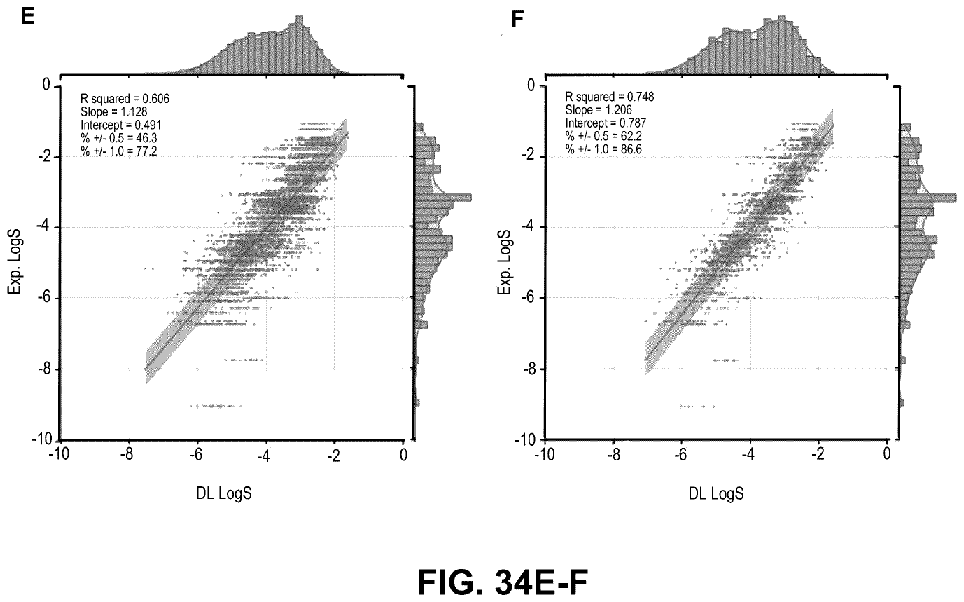

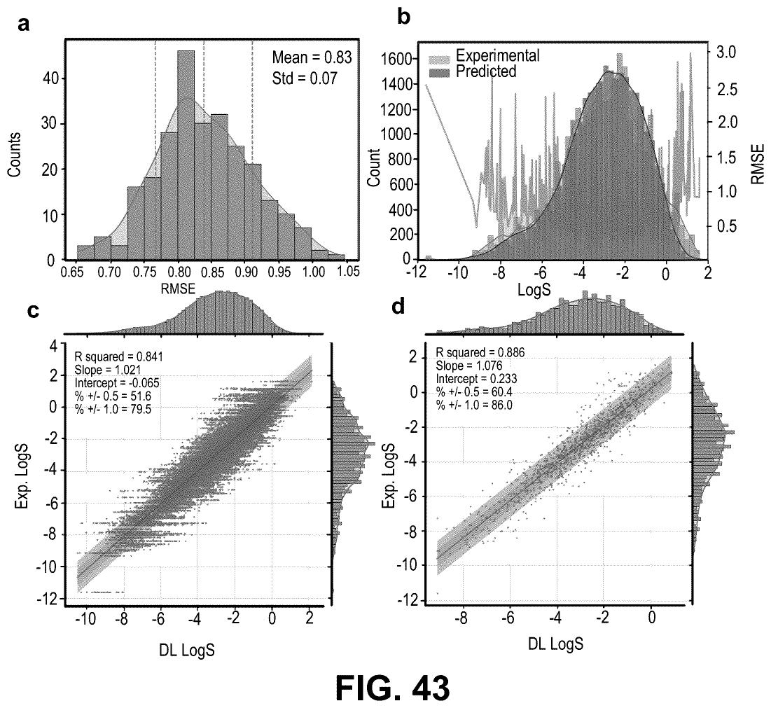

Using the closed MEMS with the same deep learning model, the number of input features per atom is ¼ of that when using four cut MEMS. The prediction results (FIG. 43a) indicate inferior performance, with R2 of 0.41 (vs. 0.61 in FIG. 32E). The numbers of molecules with half and 1 logarithmic unit are modestly fewer (39.2% and 67.5% vs. 46.0% and 77.7%), suggesting that the close MEMS could still carry sufficient chemical information of the molecules for deep learning. Serendipitously, when the same configuration of DeepSets layers were used as one used in handling four cut MEMS of each molecule, the model achieved comparable performance with the four-cut model (FIG. 34B vs. FIG. 32E). The R² value went from 0.41 to 0.61 (FIGS. 34A and 34B), almost identical to that by using four cut MEMS. The numbers of molecules with half and 1 logarithmic unit are similar as well (45.5% and 75.9%). The finding suggests that a larger neural network may help recover some of the information lost during the manifold embedding process of a closed molecular surface.

In the above exercises, Hirshfeld surface was used as molecular manifold, which is generated from the partitioning of electron densities in the crystal structure of a molecule. Further exploration used the electron-density iso-surface of a single molecule to compute MEMS and derive shape-context matrices for solubility prediction. For each molecule in the electronic calculation, the conformation remained the same as extracted from the respective crystal structure (with only the positions of hydrogen atoms optimized). The iso-surface MEMS of the same molecules from FIG. 31 show great similarities with the Hirshfeld surface counterparts. The predicted outcomes are as good as the results of using Hirshfeld surfaces. When using four cut MEMS derived from the iso-surface of each molecule, R² is 0.63 with 49.1% and 79.9% of the molecules within half and one logarithmic unit of experimental values (FIG. 33C), slightly better than the Hirshfeld model (FIG. 32E). When the data splitting went from 90:10 to 95:5 between training and testing, the predicting performance increased as well. The R² value is 0.78 with 69.2% and 92.2% of the molecules within half and one logarithmic unit of experimental values (FIG. 34D), similar to that by Hirshfeld surfaces (FIG. 27f). In addition, MEMS of closed iso-surfaces were utilized in deep learning and similar solubility prediction results were obtained (FIGS. 34E and 34F), as observed by using closed Hirshfeld surfaces (FIGS. 33A and 33B).

Lastly, fully optimized single molecules were tested for generating iso-surface MEMS and deep learning of solubility. Prediction results of the fully optimized 133 molecules resemble those by Hirshfeld surfaces or electron-density iso-surfaces that are generated from the conformations extracted from respective crystal structures. Overall, whether it was Hirshfeld surface or iso-surface, whether the surface was cut or closed for yielding MEMS, whether the molecules were fully, or partial optimized, equivalent results of solubility prediction were obtained. Moreover, 200 fully optimized molecules combined out of the two Solubility Challenges were used and utilized using their iso-surfaces for deep learning. Interestingly, compared with learning the smaller dataset of the 133 molecules, the prediction was slightly weakened. For instance, R² by utilizing four cut MEMS with 90% or 95% of molecules used in training is 0.56 or 0.72 (FIGS. 34A and 34B); the corresponding values by the 133 molecules are 0.61 and 0.77 (FIGS. 33B and 33B). Based on the distributions of experimental values, the larger dataset seems to disproportionately have a few more data points at the poor solubility tail, which may contribute to the slight decrease in prediction performance.

Chemical deep learning requires several traits to forge a robust prediction model that can approximate the latent function intended to capture. Ideally, the number of data points in the training set is sufficiently large to cover the sub-chemical space where the function resides. The quality of the experimental data used in training should be sound and well curated. The architecture of the deep learning model needs to be cleverly designed to weed out noises and uncover the salient connections among input features, facilitated by the cost function and backpropagation. More importantly, the input features that describe or represent a molecule should carry expressive and discerning information that utilizes the output data to guide the approximation of the latent function.

Many of the current schemes of molecular descriptors and fingerprints are developed from the conventional ball-and-stick notion of a molecule. In principle, if such a description scheme can fully differentiate each molecule (by uniquely projecting in an orthogonal hyperspace), the latent function between the input features and intended property should exist and the one-to-one relationship may be inferred by fitting training data. Given the underpinning nature of molecular interactions that is governed by the electronic structures of interacting molecules, nonetheless, using conventional molecular features in a deep learning exercise result in a causal function that may be too complex to develop with a suitable machine learning model, as well as too high-dimensional to fit. Such a latent function not only needs to establish the relationship(s) between the molecular representation and electronic structures but should also connect the latent electronic attributes with the molecular property of interest. The COD could be exacerbated, and model over-fitting likely occurs. Remediation requires multiple dimensionality reductions steps, explicitly or implicitly, through the machine learning process, 48 which nonetheless coarse-grains the molecular input and downgrades the model to discern molecules for prediction.

Facing the challenges, one feasible solution is to utilize the quantum chemical information of a molecule as input in the deep learning of molecular interactions and pertinent properties. Ostensibly, this could ease the aforementioned complexity due to molecular descriptors being employed. As the electronic structure and attributes of a molecule are well-defined and readily computable at various accuracy levels by quantum mechanical methods, it is viable to develop electronic features as the sole representation of a molecule for deep learning. Yet, it is difficult to directly employ electron densities and associated quantities as they are dispersed, un-structured, and dependent on rotation and translation of the molecule. Most of current efforts center on augmenting molecular graphs with electronic quantities partitioned to individual atoms or chemical bonds, or both. Graph neural networks and variants are often utilized as the deep learning architecture to numerically infer the connection between an input graph and the conforming value of property.

Taking a different route, MEMS aims to capture quantum mechanical information on a molecular surface as the molecular representation in chemical deep learning. It is conceptualized to describe a molecule by its inherent electronic attributes that govern the strength and locality of intermolecular interactions it forms. Because electron densities and associated quantities are locally distributed around the nuclei of a molecule, the electronic properties on a molecular surface or manifold are routinely utilized in understanding molecular interactions, including the local hardness and softness concepts within the framework of CDFT. To reduce the dimensionality of the electronic attributes on a surface and, equally important, to eliminate the degrees of freedom due to the positioning of the surface manifold, manifold learning was utilized and the stochastic neighbor embedding method was applied to preserve the electronic quantities in a lower dimension.

Chemical deep learning requires several traits to forge a robust prediction model that can approximate the latent function intended to capture. Ideally, the number of data points in the training set is sufficiently large to cover the sub-chemical space where the function resides. The quality of the experimental data used in training should be sound and well curated. The architecture of the deep learning model needs to be cleverly designed to weed out noises and uncover the salient connections among input features, facilitated by the cost function and backpropagation. More importantly, the input features that describe or represent a molecule should carry expressive and discerning information that utilizes the output data to guide the approximation of the latent function.

Many of the current schemes of molecular descriptors and fingerprints are developed from the conventional ball-and-stick notion of a molecule. In principle, if such a description scheme can fully differentiate each molecule (by uniquely projecting in an orthogonal hyperspace), the latent function between the input features and intended property should exist and the one-to-one relationship may be inferred by fitting training data. Given the underpinning nature of molecular interactions that is governed by the electronic structures of interacting molecules, nonetheless, using conventional molecular features in a deep learning exercise result in a causal function that may be too complex to develop with a suitable machine learning model, as well as too high-dimensional to fit. Such a latent function not only needs to establish the relationship(s) between the molecular representation and electronic structures but should also connect the latent electronic attributes with the molecular property of interest. The COD could be exacerbated, and model over-fitting likely occurs. Remediation requires multiple dimensionality reductions steps, explicitly or implicitly, through the machine learning process,48 which nonetheless coarse-grains the molecular input and downgrades the model to discern molecules for prediction.

Facing the challenges, one feasible solution is to utilize the quantum chemical information of a molecule as input in the deep learning of molecular interactions and pertinent properties. Ostensibly, this could ease the aforementioned complexity due to molecular descriptors being employed. As the electronic structure and attributes of a molecule are well-defined and readily computable at various accuracy levels by quantum mechanical methods, it is viable to develop electronic features as the sole representation of a molecule for deep learning. Yet, it is difficult to directly employ electron densities and associated quantities as they are dispersed, un-structured, and dependent on rotation and translation of the molecule. Most of current efforts center on augmenting molecular graphs with electronic quantities partitioned to individual atoms or chemical bonds, or both. Graph neural networks and variants are often utilized as the deep learning architecture to numerically infer the connection between an input graph and the conforming value of property.

Taking a different route, MEMS aims to capture quantum mechanical information on a molecular surface as the molecular representation in chemical deep learning. It is conceptualized to describe a molecule by its inherent electronic attributes that govern the strength and locality of intermolecular interactions it forms. Because electron densities and associated quantities are locally distributed around the nuclei of a molecule, the electronic properties on a molecular surface or manifold are routinely utilized in understanding molecular interactions, including the local hardness and softness concepts within the framework of CDFT. To reduce the dimensionality of the electronic attributes on a surface and, equally important, to eliminate the degrees of freedom due to the positioning of the surface manifold, manifold learning was utilized the stochastic neighbor embedding method was applied to preserve the electronic quantities in a lower dimension.

As illustrated in FIGS. 27 and 32, the MEMS of electronic attributes maintain both values and their original spatial relationships on the molecular surfaces. The same can be said about the iso-surface MEMS. The 2-D embeddings are visually vivid and authentic; they carry the totality of the quantum chemical information of a molecule pertinent to its interactions. As the eyes are more perceptive to 2-D imageries, MEMS provides readily understandable cues of the electronic features surrounding the whole molecule. Critically, MEMS is independent of the orientation or rotation of a molecule; the invariance results from the distance-based neighborhood embedding of the surface manifold. Nonetheless, because a molecular surface is enclosed, a number of false positives and negatives with regard to the manifold neighborhood are generated on the resultant MEMS. To mitigate the loss of information, manifold cutting was attempted, leading to MEMS that have no false information in light of the cut manifold, as demonstrated in FIG. 10. While the manually introduced boundary along the cutting line is correctly reproduced on the cut MEMS, false negativity is inherited from the artificial boundary points of the embedding. This may be exemplified when the Earth globe is cut and projected onto a World map—the “Far East” on one map becomes the “Central Kingdom” on another. It was posited that by using two or more cut MEMS of the same molecular surface, the inherent electron information of a molecule can be largely recovered especially by deep learning.

Electronic features such as ESP and Fukui functions bear distinctive distribution patterns on molecular surfaces and their domain sizes are commensurate with the size of an atom. ESP generally shows wider and more global features than those by Fukui functions. The true dimensionality of the electronic attributes on MEMS is thus much smaller than that of the manifold embedding (when presented as image), comparable to the number of atoms (of a molecule). The current attempt to seek the true dimensionality and thus featurize MEMS was enabled by the numerical shape context algorithm that is routinely used in computer vision. A 4×16 scheme is demonstrated in FIG. 30, where 4 angular and 16 radial bins are evaluated around a key point. The number of key points equates to the number of atoms and positioning of the key points is assigned by the closest surface points to the respective nuclei. A 1×32 scheme is also illustrated in FIG. 28. Compared to the dimensionality of an image (width×height×pixel depth), the dimensionality of a shape-context feature matrix (e.g., FIG. 11a) is significantly reduced and in line with the number of atoms. Additionally, a feature matrix is independent of positioning of MEMS (see Methods), further ensuring the electron information captured by the manifold embedding invariant of a molecule's position or rotation degrees of freedom.

The 4×16 shape-context matrices of a small but well-curated set of 133 molecules was applied in a deep-learning model of solubility prediction. DeepSets was chosen as the architecture to enable the permutation invariance of an input matrix. The MEMS of each molecule was utilized to embed several electronic properties, including ESP and Fukui functions. The prediction results support the feasibility of using MEMS and embedded electronic attributes to capture and represent a molecule in deep learning. The prediction accuracy (e.g., RMSE of each CV or RMSE of each molecule) was determined by the data distribution of training molecules. Most of the 133 molecules have logarithmic solubility values between −2.0 and −6.0, which yielded the smallest RMSE. There are only two insolubility molecules (clofazimine and terfenadine) of solubility <−7.0. Their predicted values showed the largest errors. The quality of experimental values also affected the prediction performance, demonstrated by the prediction of the 16 molecules with experimental uncertainties >0.6. While the dataset used in this study is relatively small, the close matching between the distributions of experimental data and prediction accuracy, which is seen in every deep learning exercise conducted in this study, indicates the data-fitting nature of machine learning. More crucially, the sensible robustness of the prediction accuracy to the data distribution most likely results from the inherent, quantitative connection by MEMS to solubility. The observation echoes the non-parametric nature of deep learning, which might be analogous to Gaussian Process (GP). The variance of testing data by GPs is governed by not only the variance of training data but also the covariance between the testing and training data. This might explain the significant improvement in prediction when the relative portion of testing data became smaller (i.e., 95:5 vs 90:10 of data splitting).

The prediction results even with such a small set of training data seem to support the aforesaid argument of the “domain distance” between a molecular representation and the property of interest. Such distance would be significant when a molecule is represented by a set of conventional descriptors, a graph, or a fingerprint in a model, raising the perspective of COD and requiring considerable steps of dimensionality reduction which demote the model's power to discern molecules and potentially lead to model over-fitting. In contrast, because MEMS retains the local electronic values of a molecule and their spatial relationship, the causal function between MEMS and solubility is assumed straightforward to infer by deep learning. Comparable prediction outcomes were achieved between MEMS generated from the Hirshfeld surfaces or electron-density iso-surfaces of the molecules. This suggests that a particular form of molecular surface may be irrelevant; it is the local electronic values and their spatial distribution uniquely defined by a molecule that matter. Again, if a representation scheme can differentiate molecules, it bears a latent function with the intended output, which may be approximated by a capable learning model and a set of authentic training data. This argument is echoed by the similar prediction results when the fully optimized molecules were used to generate iso-surface MEMS for the solubility prediction. Note that the insensitivity of the conformational variations (i.e., partially vs fully optimized) to solubility prediction does not suggest that the property is independent of conformation but rather due to the lack of such training data (in the present study).

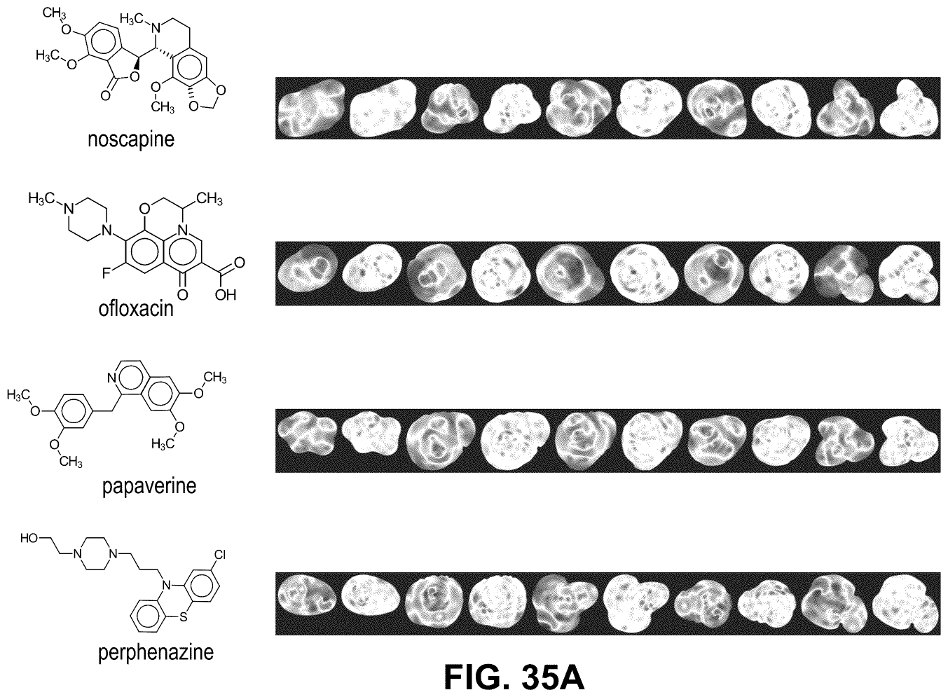

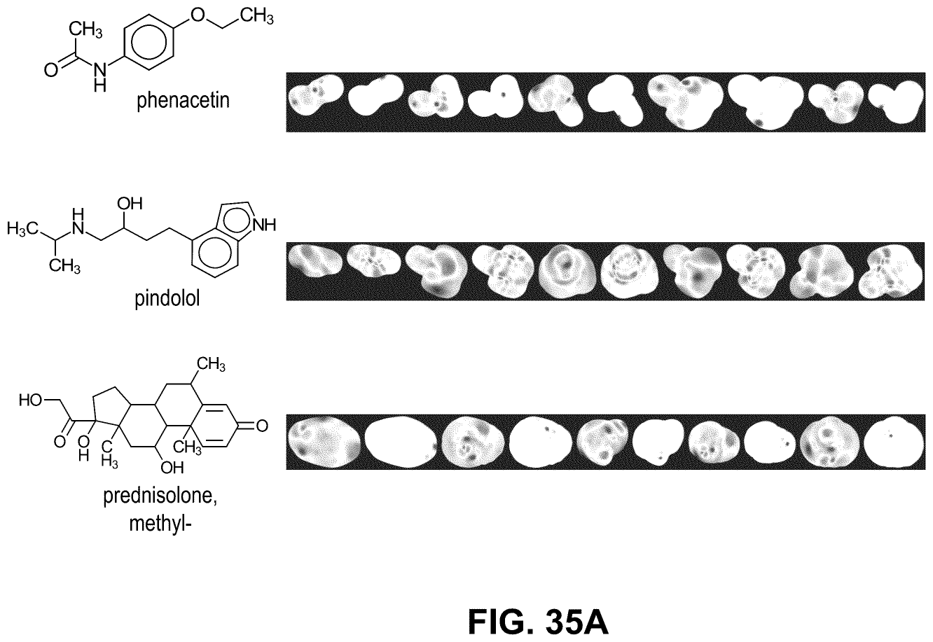

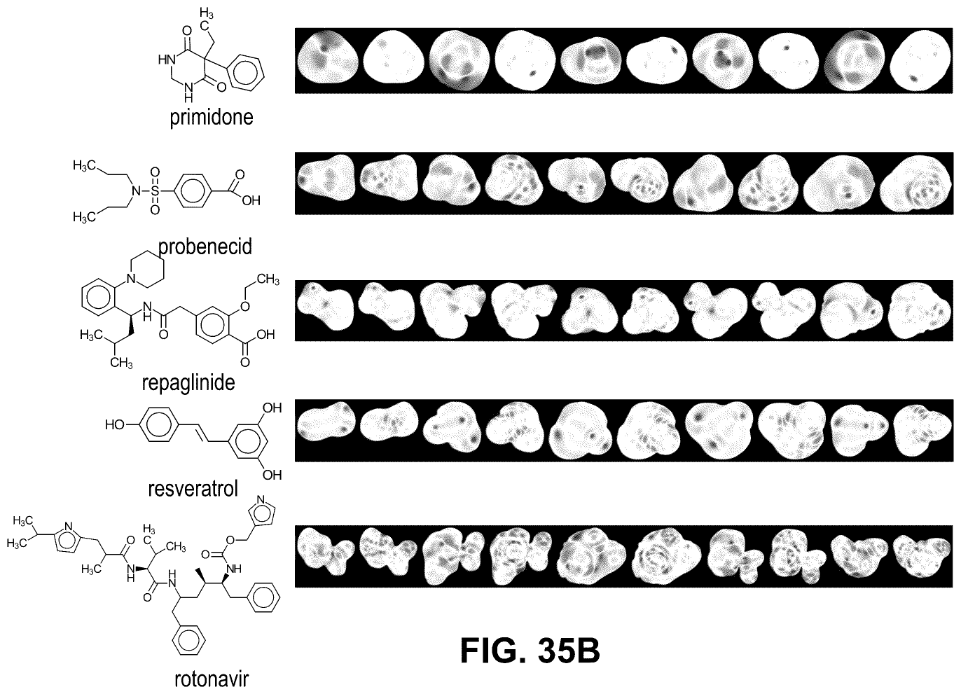

In addition, the deep-learning model consisted of multiple DeepSets layers, which implement the self-attention mechanism to derive salient features from MSMS and tie with solubility. The layers also served to implicitly reduce the dimensionalities of the learned features under the guidance of training data. Interestingly, when the width of neural networks was increased, the prediction results of using one closed MEMS to the same accuracies of using four cut MEMS of a molecule could be significantly improved (FIG. 34A vs. FIG. 34B, and FIG. 34E vs. FIG. 34F). It is noted that further improvement was insignificant when the neural network width was increased to learn the cut MEMS (data not shown), likely due to the prediction ceiling already reached (given the quality and amount of training data). The results imply that the closed MEMS could sufficiently differentiate the molecules in the dataset but bore larger uncertainties of local electronic values-compared with the joint representations by four cut MEMS of each molecule—that stemmed from the manifold embedding process. Notwithstanding, the ambiguity due to embedding electronic quantities from a manifold could be mostly overcome by additional interactions of input features, facilitated by the CONV1D operation in the deep learning model (FIGS. 35A and B).

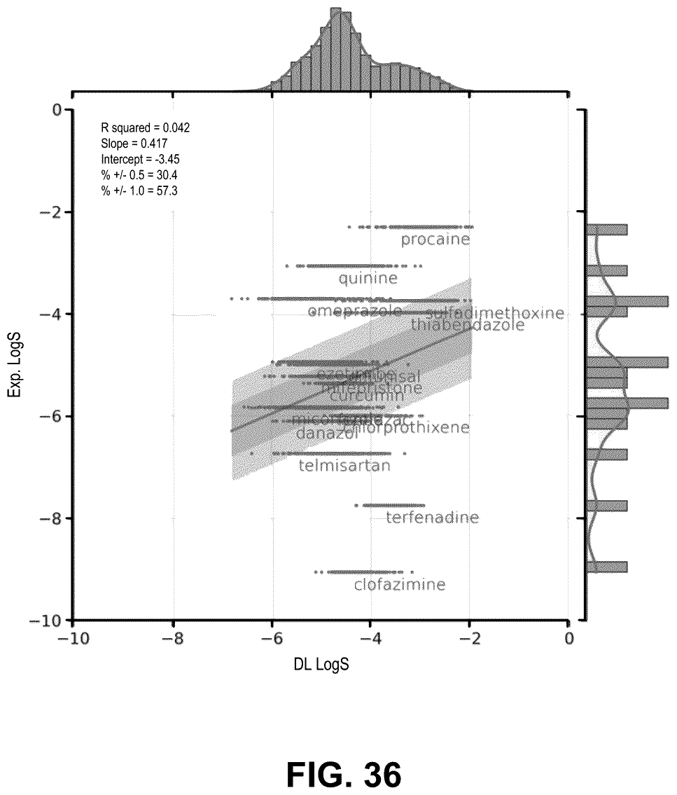

With suitable coverage and resolution, a larger training set is expected to improve prediction accuracy. However, the improvement was not observed when 200 fully optimized molecules were utilized, compared with the trials of the 133 molecules (FIGS. 33A-H and FIGS. 34A-F). The slight decrease in prediction outcome could result from a heavier tail on the poor solubility side of the experimental data distribution (FIGS. 35A and B). Additionally, the quality of experimental data can dramatically affect the discerning capabilities and prediction performance, as illustrated by the prediction of 16 molecules with larger experimental errors (FIG. 36). Clearly, a much larger dataset is needed to reduce the large distribution in prediction accuracy, which is indicated by RMSE of CVs and molecules, and to widen the prediction range.

Although direct comparison may not be objective due to different methods and training datasets being used, the predicted results seem to outperform the three dozen attempts reported in the two Solubility Challenges. Various machine learning models were employed, including Random Forest, Supporting Vector Machine, Gaussian Process, and neural networks. The size of training datasets ranged from 81 to 10,237 molecules. One top performer trained with more than 2,000 molecules by RBF (radial basis function) had the best RMSE of 0.78 and R2 of 0.62 with 54% of molecules within half logarithmic unit from respective experimental values. If the average predicted values is used instead of individual ones to fit against experimental data, the best R² would be 0.90 in FIG. 27i (with 81.1 and 98.2% of molecules within half and 1 logarithmic unit). While this comparison is superficial, the molecular representations used by the contestants were mostly based on structural features of molecules, which might contribute to the broad variations in prediction performance and difficulties of developing effective machine learning models, as alluded to earlier.

Lastly, a benchmarking study of solubility prediction was conducted using the ESOL dataset that has 1128 molecules. The results show that the deep-learning approach could achieve competitive performance. The best RMSE value averaged over 256 CVs is 0.73 and 0.64 over individual molecules. The R² of the predicted versus experimental values is 0.88 and that by the averaged predicted values is 0.91. Conversely, MoleculeNet reports several deep learning models on ESOL and the best RMSE value of 0.58, which is produced by a graph convolution model, MPNN (message passing neural network).53 A more recent study that benchmarks molecular representations, including fingerprints, descriptors, and graph neural networks, on several datasets of molecular properties achieved a better RMSE, 0.56, on ESOL by D-MPNN (directed MPNN).4 It is however indicated by this study that the impressive RMSE might result from model over-fitting because of the random split of the dataset used for training and testing. As graph convolution tends to extract local features of a molecule, such a deep learning model may memorize molecular scaffolds shared between training and testing data rather than generalize the chemistry from the data. It is further suggested that scaffold split of the dataset is a more robust measure for performance evaluation (of a graph convolution model), which led to the best RMSE of 0.99.4 Another study that also utilized scaffold split and a geometry-based GNN model resulted in its best value of 0.80.54 Noted in these GNN studies is the dataset typically split just three times, either randomly or based on scaffold. In the present approach, MEMS captures both global and local features of electronic attributes that directly determine molecular interactions and running multiple cross validations should provide a thorough assessment of prediction performance, especially from comparing the distributions of predicted and experimental values.

The RMSE of individual molecules in ESOL prediction remain largely invariant to the distribution of experimental data, much different from what is illustrated in the Solubility Challenge predictions (e.g., FIGS. 9b, e, and h), where more precise prediction is enabled around the peak of the data distribution. Under the lens of Gaussian Process, the RMSE vs. data distribution suggests that the solubility data in ESOL bears much larger variations or experimental errors than the data in Solubility Challenges. One possible cause could be the solubility values of many weak acids or bases in ESOL not corrected for pH.55 Another interesting finding from the ESOL prediction is that the slopes of the prediction versus experimental values are close to 1.0, suggesting that the deep learning model works equally well along the solubility range, albeit limited by the quality of the experimental data. The same finding can also be made from the Solubility Challenge predictions (e.g., FIG. 15).

A new concept of molecular representation was thus developed to preserve quantum information of electronic attributes in a lower-dimensional embedding. The idea originated from the earlier studies of evaluating molecular interactions with local hardness and softness quantities within the CDFT framework. The electronic features extracted from MEMS seem to capture the totality of a molecule's inherent capability to interact with another molecule, both the strength and locality. Based on solubility prediction, it appears MEMS uniquely represents a molecule (or a conformer, to be specific) and, more importantly, enables the development of robust deep learning models that sensibly connect with molecular properties. By utilizing much more training data in future studies, it was expected that the MEMS approach will lead to generalized, practical models. Because MEMS carries no direct information of the underlying molecular structure (elemental composition and chemical bonding) but local electronic quantities at the boundary of a molecule, using the concept in deep learning could overcome the so-called issue of activity cliffs, where a minor structural change results in a significant difference in the activity of interest that is likely reflective of substantial variations in electronic attributes on molecular surface. As it undertakes a mathematical formality of manifold learning and the chemical foundation of molecular interactions by the HSAB principle and CDFT, MEMS seems to ease the development of deep learning architectures and lessen the challenges due to the COD in data-driven chemical learning.

It is worth noting that several efforts have been reported to capture or featurize electronic attributes or chemical interaction quantities on a molecular surface for predicting molecular similarities or properties. One earlier study was the development of self-organizing maps (SOMs) of molecules, where surface points are mapped to a regularly spaced 2-D grid based on neighborhood probabilities, which are shown similarly defined to those used in SNE. Spatial autocorrelation of electronic properties on a molecular surface was attempted, leading to a number of autocorrelation coefficients to be utilized in QSAR studies. In the COSMO-RS approach, which is widely utilized in predicting a small molecule's solubility in another solvent, the screening charge densities on a molecular surface are partitioned as a probability distribution profile and employed in the prediction. More recently, electronic attributes and several other chemical and geometric properties on a protein surface were directly featurized by a geodesic CNN approach and used in deep learning of protein interactions. Specifically, a patch of neighboring points on triangulated mesh is aggregated for a surface vertex by applying a Gaussian kernel with trainable parameters (mean and variance) defined by local geodesic and polar coordinates. For each vertex, multiple Gaussian kernels may be applied for convoluting surface properties, leading to a multi-dimensional, trainable fingerprint used in deep learning. A similar effort circumvented the mesh triangulation step and directly conducted geometric convolution on the point cloud of a protein surface. It seems, however, in these geometric deep learning efforts that rotational invariance is numerically handled by using multiple orientations of a surface in training. It would be interesting to see how geometric CNN performs on small molecules. Compared with these efforts, especially the last one, ours may be regarded as unsupervised learning by manifold embedding and shape-context featurization, followed by supervised deep learning.

Manifold Embedding of Molecular Surface (MEMS) is one way to represent electronic attributes of a molecule. Broadly speaking, the electronic densities and associated properties (such as electrostatic potential, ESP, and Fukui functions) of a molecule are un-structured and thus not suitable for machine learning. This is the result of at least two factual traits: (1) the amount of data with associated dimensions is enormous; and (2) the representation of these data may be rotation- and translation-dependent. MEMS attempts to capture quantum information on a molecular surface, which is extended by learning kernel representations of manifold embeddings via Gaussian Process. Preliminary results suggest that MEMS kernels are more expressive than shape-context matrices in recovering the chemical information of the “underlying” molecule. This also allows the use of MEMS kernels for solubility and other chemical property predictions.

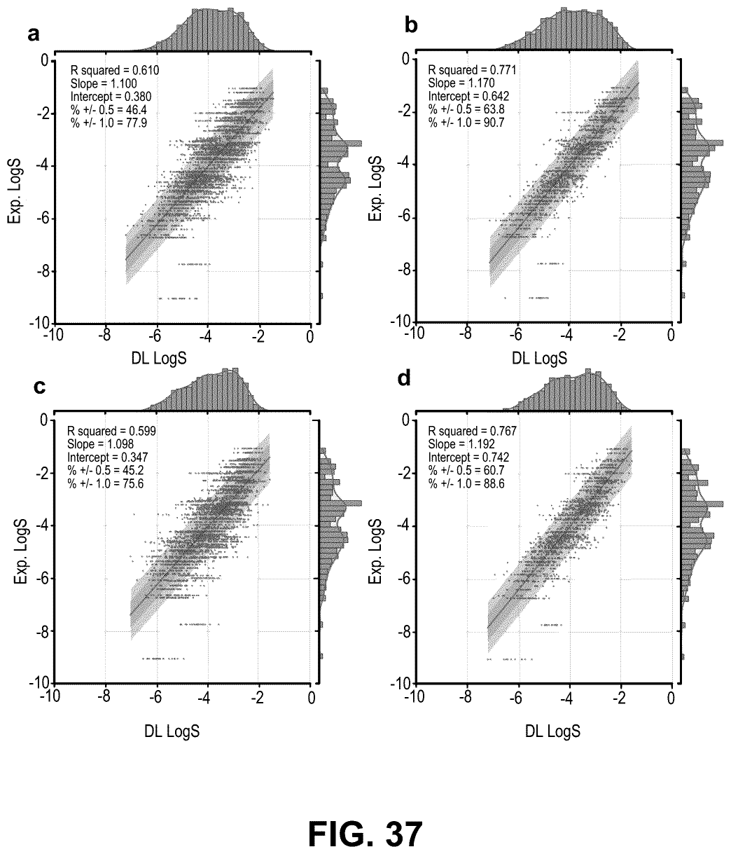

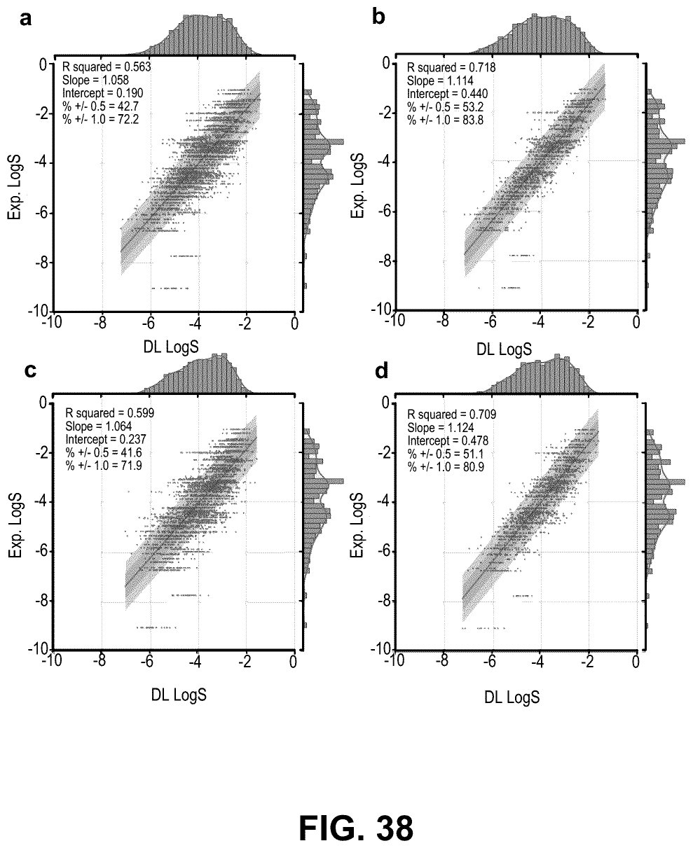

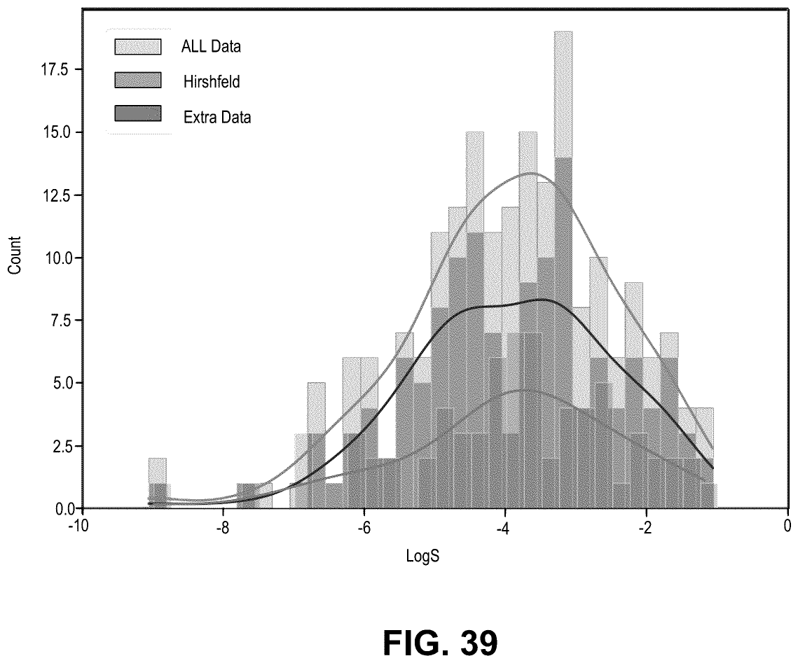

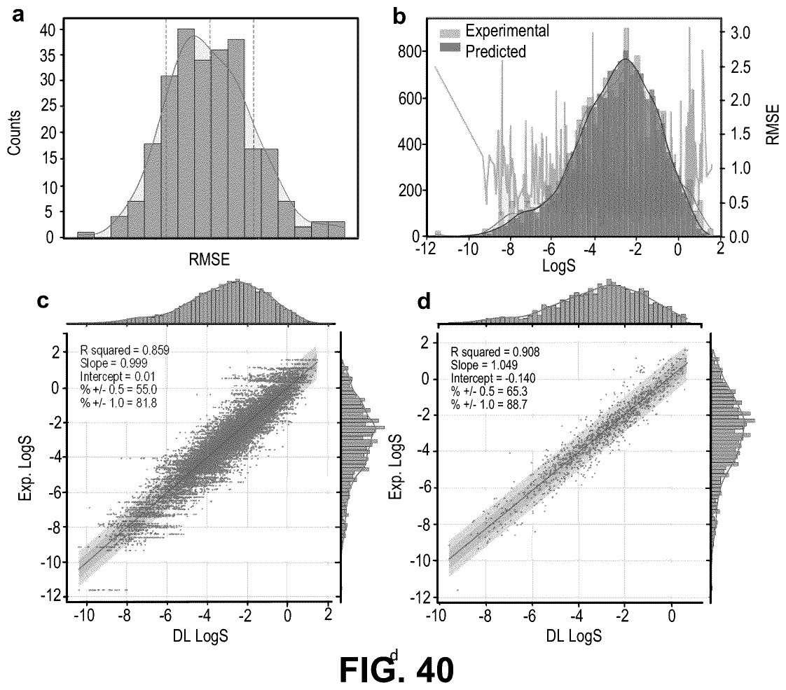

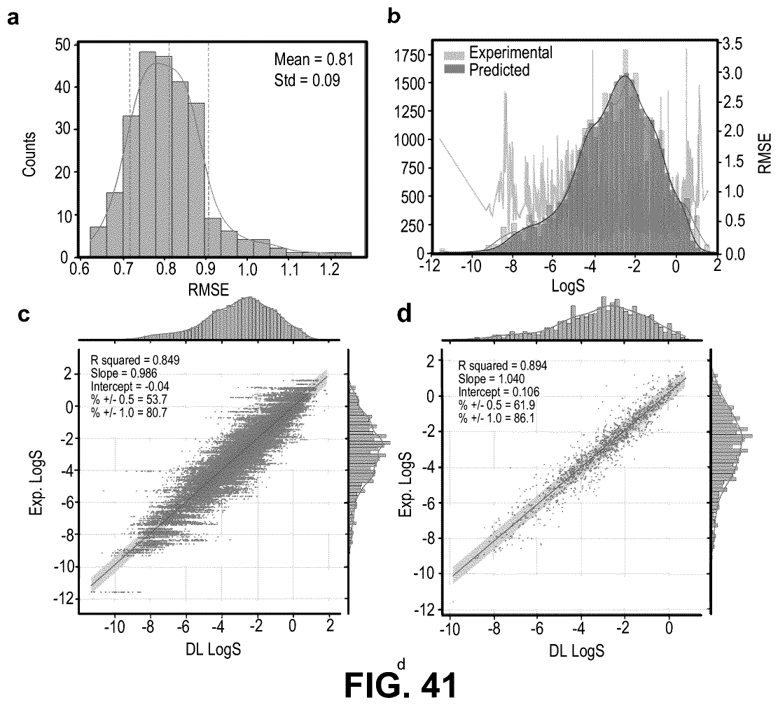

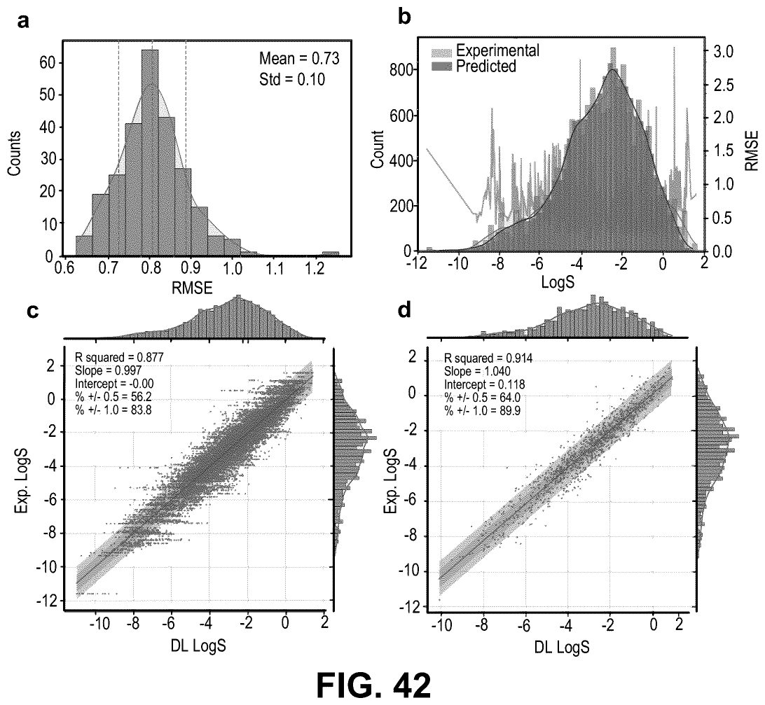

For example, FIG. 36 shows predicted vs. experimental solubility logarithm values of the 16 molecules in the second set of the second Solubility Challenge. Training was conducted with the remaining 117 molecules and repeated 64 times to keep the prediction results that had the smallest MAE. Shape-context matrices of four cut MEMS were utilized. FIG. 37 and FIG. 38 show prediction versus experimental values by iso-surface MEMS of 133 fully optimized single molecules: four cut MEMS of each molecule (37a and 38a), four cut trained with 95% of molecules (37b and 38b), closed MEMS (37c and 38c), and closed MEMS trained with larger DeepSets layers (31d and 32d). FIG. 39 shows data distributions of experimental values of solubility of all 200 molecules, 133 molecules with crystal structures and the extra 67 molecules without a proper crystal structure. FIG. 40 has four charts of Prediction results of ESOL by four cut iso-surface MEMS at 90:10 split of the dataset for training and testing, RMSE of cross validations (40a), overlapped distributions of the predicted (blue) and experimental values (red) of Log S with individual RMSE values of the molecules plotted as green line (40b), predicted versus experimental values of individual molecules (40c), and mean predicted versus experimental values of individual molecules (40d). Note that the same computation and deep learning procedures as for the Solubility Challenge dataset were applied to ESOL. Of the 1128 molecules, 20 were excluded due to quantum mechanical computation difficulties by the basis set of molecule optimization. The 20 molecules include 16 with iodine and 4 with sulfur atom. Cross validations (256) were conducted and each random split of the dataset was repeated 12 times by re-initializing the weights of the deep learning model. FIG. 41 has a further four charts of prediction results of ESOL by four cut iso-surface MEMS at 95:5 split of the dataset for training and testing, RMSE of cross validations (41a), overlapped distributions of the predicted (blue) and experimental values (red) of Log S with individual RMSE values of the molecules plotted as green line (41b), predicted versus experimental values of individual molecules (41c), and mean predicted versus experimental values of individual molecules (41d). FIG. 42 has prediction results of ESOL by closed iso-surface MEMS at 90:10 split of the dataset for training and testing, RMSE of cross validations (42a), overlapped distributions of the predicted (blue) and experimental values (red) of Log S with individual RMSE values of the molecules plotted as green line (42b), predicted versus experimental values of individual molecules (42c), and mean predicted versus experimental values of individual molecules (42d). FIG. 43 has prediction results of ESOL by closed iso-surface MEMS at 95:5 split of the dataset for training and testing, RMSE of cross validations (43a), overlapped distributions of the predicted (blue) and experimental values (red) of Log S with individual RMSE values of the molecules plotted as green line (43b), predicted versus experimental values of individual molecules (43c), and mean predicted versus experimental values of individual molecules (43d).

In developing the instant disclosure, the following compounds of Table 3 were used:

TABLE 3
Molecules Used in the Deep Learning of Drug Solubilities to
Generate Hirshfeld Surfaces and Electron-density Iso-surfaces.

| Compound | Ref Code | Data Source | LogS | Index |
|---|---|---|---|---|
| acetaminophen | HXACAN04 | 2008 Set1 | −1.06 | 0 |
| atropine | BIHWEX | 2008 Set1 | −2 | 7 |
| azathioprine | CIPWUT01 | 2008 Set1 | −3.21 | 8 |
| benzylimidazole | EYIRIN01 | 2008 Set1 | −2.26 | 12 |
| bromogramine | EVIDIY | 2008 Set1 | −4.06 | 14 |
| bupivacaine | KEFFUZ | 2008 Set1 | −3.22 | 15 |
| carprofen | UQOBIM | 2008 Set1 | −4.7 | 19 |
| carvedilol | GIVJUQ | 2008 Set1 | −4.26 | 20 |
| chlorpromazine | CPROMZ | 2008 Set1 | −5.07 | 22 |
| diphenylhydantoin | PHYDAN03 | 2008 Set1 | −3.86 | 43 |
| enrofloxacin | XICPAC | 2008 Set1 | −3.18 | 45 |
| famotidine | FOGVIG01 | 2008 Set1 | −2.65 | 50 |
| flufenamic acid | FPAMCA11 | 2008 Set1 | −5.35 | 52 |
| guanine | KEMDOW | 2008 Set1 | −4.43 | 58 |
| hexobarbital | DMCYBA | 2008 Set1 | −2.67 | 60 |
| hydroflumethiazide | EWUHAF | 2008 Set1 | −2.97 | 62 |
| hydroxybenzoic acid | JOZZIH01 | 2008 Set1 | −1.46 | 63 |
| ibuprofen | IBPRAC | 2008 Set1 | −3.59 | 64 |
| mefenamic acid | XYANAC | 2008 Set1 | −6.74 | 70 |
| metoclopramide | METPRA | 2008 Set1 | −3.57 | 71 |
| metronidazole | MNIMET | 2008 Set1 | −1.22 | 72 |
| nalidixic acid | NALIDX | 2008 Set1 | −3.61 | 76 |
| naphthol | NAPHOL01 | 2008 Set1 | −1.98 | 78 |
| niflumic acid | NIFLUM11 | 2008 Set1 | −4.58 | 82 |
| nitrofurantoin | LABJON | 2008 Set1 | −3.24 | 84 |
| oxytetracycline | OXYTET | 2008 Set1 | −3.09 | 89 |
| phenobarbital | PHBARB | 2008 Set1 | −2.29 | 93 |
| phenylbutazone | JAJLIQ | 2008 Set1 | −4.39 | 94 |
| phthalic acid | PHTHAC06 | 2008 Set1 | −1.61 | 95 |
| piroxicam | BIYSEH | 2008 Set1 | −4.8 | 97 |
| propranolol | PROPRA10 | 2008 Set1 | −3.49 | 102 |
| ranitidine | POPFAC | 2008 Set1 | −2.5 | 105 |
| sparfloxacin | JEKMOB | 2008 Set1 | −3.37 | 111 |
| sulfacetamide | SLFNMG01 | 2008 Set1 | −1.52 | 114 |
| sulfamethazine | SLFNMD11 | 2008 Set1 | −2.73 | 117 |
| thymol | IPMEPL | 2008 Set1 | −2.19 | 128 |
| trichloromethiazide | KIKCUD | 2008 Set1 | −3.53 | 130 |
| trimethoprim | AMXBPM12 | 2008 Set1 | −2.95 | 131 |
| tryptamine | XUDTOF | 2008 Set1 | −3.3 | 132 |
| benzocaine | QQQAXG02 | 2008 Set2 | −2.33 | 10 |
| clozapine | NDNHCL10 | 2008 Set2 | −3.24 | 30 |
| dibucaine | WADTIG | 2008 Set2 | −4.39 | 37 |
| diethylstilbestrol | ESTILO02 | 2008 Set2 | −4.43 | 39 |
| furosemide | FURSEM14 | 2008 Set2 | −4.23 | 54 |
| hydrochlorothiazide | HCSBTZ | 2008 Set2 | −2.68 | 61 |
| ketoprofen | KEMRUP | 2008 Set2 | −3.21 | 68 |
| meclofenamic acid | MECLOF12 | 2008 Set2 | −6.27 | 69 |
| naphthoic acid | NAPHAC | 2008 Set2 | −3.77 | 77 |
| pyrimethamine | MUFMAB01 | 2008 Set2 | −4.11 | 103 |
| salicylic acid | SALIAC | 2008 Set2 | −1.93 | 110 |
| sulfamerazine | SLFNMA01 | 2008 Set2 | −3.12 | 116 |
| sulfamethizole | AMUTOT | 2008 Set2 | −2.78 | 118 |
| tolbutamide | ZZZPUS02 | 2008 Set2 | 3.46 | 129 |
| acetazolamide | ATDZSA | 2019 Set1 | −2.38 | 1 |
| acetylsalicylic acid | ACSALA | 2019 Set1 | −1.67 | 2 |
| alclofenac | FICJAC | 2019 Set1 | −4.4 | 3 |
| ambroxol | FIYBEV01 | 2019 Set1 | −3.87 | 4 |
| aripiprazole | MELFIT01 | 2019 Set1 | −6.64 | 5 |
| atovaquone | UMOMAL02 | 2019 Set1 | −6.07 | 6 |
| baclofen | AQEKUE | 2019 Set1 | −1.78 | 9 |
| benzthiazide | AMEVAP | 2019 Set1 | −4.84 | 11 |
| bromazepam | CAGWOW | 2019 Set1 | −3.39 | 13 |
| carbamazepine | CBMZPN10 | 2019 Set1 | −3.22 | 16 |
| carbazole | CRBZOL01 | 2019 Set1 | −5.19 | 17 |
| carbendazim | SEDZUW01 | 2019 Set1 | −4.56 | 18 |
| celecoxib | DIBBUL | 2019 Set1 | −5.89 | 21 |
| chlorpropamide | BEDMIG | 2019 Set1 | −3.17 | 23 |
| cholic acid | EDEBOE | 2019 Set1 | −4.62 | 25 |
| cilostazol | XOSGUH | 2019 Set1 | −4.93 | 26 |
| cimetidine | CIMETD | 2019 Set1 | −1.52 | 27 |
| ciprofloxacin | UHITOV | 2019 Set1 | −3.57 | 28 |
| corticosterone | CORTIC | 2019 Set1 | −3.29 | 31 |
| cortisoneacetate | ACPRET | 2019 Set1 | −4.22 | 32 |
| daidzein | XEKCUO | 2019 Set1 | −5.23 | 34 |
| diazoxide | DIAZOX | 2019 Set1 | −3.43 | 36 |
| diclofenac | SIKLIH | 2019 Set1 | −5.34 | 38 |
| diflorasone diacetate | IHAPOX | 2019 Set1 | −4.82 | 40 |
| diltiazem | GAJKAG | 2019 Set1 | −3.02 | 42 |
| DOPA | LDOPAS11 | 2019 Set1 | −1.76 | 44 |
| estradiol | HXESDO | 2019 Set1 | −5 | 46 |
| estrone | ESTRON10 | 2019 Set1 | −5.38 | 47 |
| eucalyptol | MOFPAY | 2019 Set1 | −1.66 | 48 |
| flurbiprofen | FLUBIP | 2019 Set1 | −4.34 | 53 |
| ganciclovir | UGIVAI | 2019 Set1 | −1.78 | 55 |
| glipizide | SAXFED | 2019 Set1 | −5.61 | 56 |
| griseofulvin | GRISFL | 2019 Set1 | −4.52 | 57 |
| haloperidol | HALDOL01 | 2019 Set1 | −5.71 | 59 |
| indomethacin | INDMET | 2019 Set1 | −5.48 | 65 |
| indoprofen | LEKMET | 2019 Set1 | −4.65 | 66 |
| ketoconazole | KCONAZ | 2019 Set1 | −5.47 | 67 |
| nabumetone | XOCXUI02 | 2019 Set1 | −4.4 | 75 |
| naproxen | COYRUD | 2019 Set1 | −4.23 | 79 |
| nevirapine | PABHIJ | 2019 Set1 | −3.41 | 80 |
| nifedipine | BICCIZ | 2019 Set1 | −4.71 | 81 |
| nimesulide | WINWUL | 2019 Set1 | −4.74 | 83 |
| norfloxacin | VETVOG | 2019 Set1 | −2.88 | 85 |
| noscapine | BOVPUA | 2019 Set1 | −4.48 | 86 |
| ofloxacin | CUYCEF | 2019 Set1 | −2.03 | 87 |
| papaverine | MVERIQ01 | 2019 Set1 | −4.33 | 90 |
| perphenazine | PERPAZ | 2019 Set1 | −4.48 | 91 |
| phenacetin | PYRAZB21 | 2019 Set1 | −2.3 | 92 |
| pindolol | PINDOL | 2019 Set1 | −3.75 | 96 |
| prednisolone, methyl- | MTHPRG | 2019 Set1 | −3.33 | 98 |
| primidone | EPHPMO | 2019 Set1 | −2.53 | 99 |
| probenecid | QQQBSS03 | 2019 Set1 | −4.83 | 100 |
| repaglinide | JOHKUM | 2019 Set1 | −4.77 | 106 |
| resveratrol | DALGON | 2019 Set1 | −3.75 | 107 |
| ritonavir | YIGPIO03 | 2019 Set1 | −5.17 | 108 |
| rofecoxib | CAXMUJ | 2019 Set1 | −4.61 | 109 |
| spironolactone | ATPRCL10 | 2019 Set1 | −4.21 | 112 |
| strychnine | ZZZUEE01 | 2019 Set1 | −3.38 | 113 |
| sulfasalazine | QIJZOY | 2019 Set1 | −6.41 | 119 |
| sulfathiazole | SUTHAZ | 2019 Set1 | −2.62 | 120 |
| sulfisomidine | SLFSMD | 2019 Set1 | −2.16 | 121 |
| sulfisoxazole | SLFSXZ10 | 2019 Set1 | −3.13 | 122 |
| sulindac | DOHREX | 2019 Set1 | −4.96 | 123 |
| thiacetazone | GANGEH | 2019 Set1 | −3.5 | 127 |
| chlorprothixene | CMAPTX | 2019 Set2 | −5.99 | 24 |
| clofazimine | DAKXUI | 2019 Set2 | −9.05 | 29 |
| curcumin | BINMEQ02 | 2019 Set2 | −5.36 | 33 |
| danazol | YAPZEU01 | 2019 Set2 | −6.1 | 35 |
| diflunisal | FAFWIS01 | 2019 Set2 | −4.99 | 41 |
| ezetimibe | QUWYIR | 2019 Set2 | −4.94 | 49 |
| fentiazac | PCPTZA | 2019 Set2 | −5.84 | 51 |
| miconazole | PAVPIP | 2019 Set2 | −5.82 | 73 |
| mifepristone | ZIDLED | 2019 Set2 | −5.22 | 74 |
| omeprazole | VAYXOI | 2019 Set2 | −3.7 | 88 |
| procaine | BEWYIL01 | 2019 Set2 | −2.3 | 101 |
| quinine | BOMDUC | 2019 Set2 | −3.06 | 104 |
| sulfadimethoxine | SFDMOX | 2019 Set2 | −3.74 | 115 |
| telmisartan | XUYHOO | 2019 Set2 | −6.73 | 124 |
| terfenadine | EWEMIF | 2019 Set2 | −7.74 | 125 |
| thiabendazole | THBDAZ10 | 2019 Set2 | −3.97 | 126 |

In addition to the information listed in the above table, FIG. 29 provides the MEMS representation of the first 13 listed compounds above.

In contrast to MEMS, the present MKMS process directly kernelizes the electronic attributes on a molecular surface without going through the embedding process, thereby avoiding information loss due that can result from embedding. The manifold kernels are thus more accurate and salient in retaining the electronic information of a molecule, especially in generative deep learning for de novo design of molecules. In that regard, MKMS offers (1) information completeness, i.e., no information loss; (2) robustness against dimensionality reduction; and (3) mathematically differentiable, which can be important for generative AI prediction of molecular structures.

The nature of deep learning lies in generalization out of the training data. However, a model may not generalize outside of the chemistry within its training data. This is because the weights in a neural network are deduced from optimization of the loss function by the training data. Altogether, the tight-knit connections among molecular description, model architecture, quality and quantity of training data play collective roles in determining the prediction capability of a chemical learning effort.

The following references were used in the development of the present invention, and the disclosures of which are explicitly incorporated by reference herein:
1. Weininger D. Smiles, a Chemical Language and Information-System.1. Introduction to Methodology and Encoding Rules. J Chem Inf Comput Sci 28, 31-36 (1988).
2. Rogers D, Hahn M. Extended-Connectivity Fingerprints. J Chem Inf Model 50, 742-754 (2010).
3. Jiang D, Wu Z, Hsieh C Y, Chen G, Liao B, Wang Z, Shen C, Cao D, Wu J, Hou T. Could Graph Neural Networks Learn Better Molecular Representation for Drug Discovery? A Comparison Study of Descriptor-Based and Graph-Based Models. J Cheminform 13, 12 (2021).
4. Yang K, Swanson K, Jin W G, Coley C, Eiden P, Gao H, Guzman-Perez A, Hopper T, Kelley B, Mathea M, Palmer A, Settels V, Jaakkola T, Jensen K, Barzilay R. Analyzing Learned Molecular Representations for Property Prediction. J Chem Inf Model 59, 3370-3388 (2019).
5. Bellman R E. Adaptive Control Processes. Princeton University Press (1961).
6. Hughes G F. On Mean Accuracy of Statistical Pattern Recognizers. IEEE Trans Inf Theory 14, 55-63 (1968).
7. Randic M. Orthogonal Molecular Descriptors. New J Chem 15, 517-525 (1991).
8. Racz A, Bajusz D, Heberger K. Intercorrelation Limits in Molecular Descriptor Preselection for QSAR/QSPR. Mol Inform 38, (2019).
9. Thanikaivelan P, Subramanian V, Rao J R, Nair B U. Application of Quantum Chemical Descriptor in Quantitative Structure Activity and Structure Property Relationship. Chem Phys Lett 323, 59-70 (2000).
10. Rupp M, Tkatchenko A, Muller K R, von Lilienfeld O A. Fast and Accurate Modeling of Molecular Atomization Energies with Machine Learning. Phys Rev Lett 108, (2012).
11. Matta C F. Molecules as Networks: A Localization-Delocalization Matrices Approach. Comput Theor Chem 1124, 1-14 (2018).
12. Bader R F W. Atoms in Molecules. Acc Chem Res 18, 9-15 (1985).
13. Bader R F W. A Bond Path: A Universal Indicator of Bonded Interactions. J Phys Chem A 102, 7314-7323 (1998).
14. Kneiding H, Lukin R, Lang L, Reine S, Pedersen T B, De Bin R, Balcells D. Deep Learning Metal Complex Properties with Natural Quantum Graphs. Digital Discovery 2, 618-633 (2023).
15. Glendening E D, Landis C R, Weinhold F. Natural Bond Orbital Methods. Wiley Interdiscip Rev Comput Mol Sci 2, 1-42 (2012).
16. Qiao Z, Christensen A S, Welborn M, Manby F R, Anandkumar A, Miller T F, 3rd. Informing Geometric Deep Learning with Electronic Interactions to Accelerate Quantum Chemistry. Proc Natl Acad Sci USA 119, e2205221119 (2022).
17. Wu Z H, Pan S R, Chen F W, Long G D, Zhang C Q, Yu P S. A Comprehensive Survey on Graph Neural Networks. IEEE Trans Neural Netw Learn Syst 32, 4-24 (2021).
18. Gavezzotti A. Calculation of Lattice Energies of Organic Crystals: The Pixel Integration Method in Comparison with More Traditional Methods. Z Kristall 220, 499-510 (2005).
19. Cramer R D, Patterson D E, Bunce J D. Comparative Molecular-Field Analysis (COMFA).1. Effect of Shape on Binding of Steroids to Carrier Proteins. J Am Chem Soc 110, 5959-5967 (1988).
20. Li T L, Liu S B, Feng S X, Aubrey C E. Face-Integrated Fukui Function: Understanding Wettability Anisotropy of Molecular Crystals from Density Functional Theory. J Am Chem Soc 127, 1364-1365 (2005).
21. Li T L. Understanding the Large Librational Motion of the Methyl Group in Aspirin and Acetaminophen Crystals: Insights from Density Functional Theory. Cryst Growth Des 6, 2000-2003 (2006).
22. Li T L, Ayers P W, Liu S B, Swadley M J, Aubrey-Medendorp C. Crystallization Force-a Density Functional Theory Concept for Revealing Intermolecular Interactions and Molecular Packing in Organic Crystals. Chem Eur J 15, 361-371 (2009).
23. Mattei A, Li T L. Interplay between Molecular Conformation and Intermolecular Interactions in Conformational Polymorphism: A Molecular Perspective from Electronic Calculations of Tolfenamic Acid. Int J Pharm 418, 179-186 (2011).
24. Zhou P P, Ayers P W, Liu S B, Li T L. Natural Orbital Fukui Function and Application in Understanding Cycloaddition Reaction Mechanisms. Phys Chem Chem Phys 14, 9890-9896 (2012).
25. Zhang M T, Li T L. Intermolecular Interactions in Organic Crystals: Gaining Insight from Electronic Structure Analysis by Density Functional Theory. Crystengcomm 16, 7162-7171 (2014).
26. Bhattacharjee R, Verma K, Zhang M, Li T L. Locality and Strength of Intermolecular Interactions in Organic Crystals: Using Conceptual Density Functional Theory (CDFT) to Characterize a Highly Polymorphic System. Theor Chem Acc 138, 121 (2019).
27. Pearson R G. Hard and Soft Acids and Bases. J Am Chem Soc 85, 3533-3539 (1963).
28. Pearson R G. Acids and Bases. Science 151, 172-177 (1966).
29. Parr R G, Donnelly R A, Levy M, Palke W E. Electronegativity-Density Functional Viewpoint. J Chem Phys 68, 3801-3807 (1978).
30. Chattaraj P K, Lee H, Parr R G. Hsab Principle. J Am Chem Soc 113, 1855-1856 (1991).
31. Geerlings P, De Proft F, Langenaeker W. Conceptual Density Functional Theory. Chem Rev 103, 1793-1873 (2003).
32. Ayers P W, Liu S B, Li T L. Chargephilicity and Chargephobicity: Two New Reactivity Indicators for External Potential Changes from Density Functional Reactivity Theory. Chem Phys Lett 480, 318-321 (2009).
33. Liu S B, Li T L, Ayers P W. Potentialphilicity and Potentialphobicity: Reactivity Indicators for External Potential Changes from Density Functional Reactivity Theory. J Chem Phys 131, 114106 (2009).
34. Tenenbaum J B, de Silva V, Langford J C. A Global Geometric Framework for Nonlinear Dimensionality Reduction. Science 290, 2319-2323 (2000).
35. Law M H C, Jain A K. Incremental Nonlinear Dimensionality Reduction by Manifold Learning. IEEE PAMI 28, 377-391 (2006).
36. Lin T, Zha H B. Riemannian Manifold Learning. IEEE PAMI 30, 796-809 (2008).
37. van der Maaten L, Hinton G. Visualizing Data Using T-SNE. J Mach Learn Res 9, 2579-2605 (2008).
38. Venna J, Peltonen J, Nybo K, Aidos H, Kaski S. Information Retrieval Perspective to Nonlinear Dimensionality Reduction for Data Visualization. J Mach Learn Res 11, 451-490 (2010).
39. Spackman M A, Jayatilaka D. Hirshfeld Surface Analysis. Crystengcomm 11, 19-32 (2009).
40. Richards F M. Areas, Volumes, Packing, and Protein-Structure. Annu Rev Biophys Bioeng 6, 151-176 (1977).
41. Mattei A, Li T. Nucleation of Conformational Polymorphs: A Computational Study of Tolfenamic Acid by Explicit Solvation. Cryst Growth Des 14, 2709-2713 (2014).
42. Belongie S, Mori G, Malik J. Matching with Shape Contexts. Stat Anal Shapes, 81-105 (2006).
43. Rubner Y, Tomasi C, Guibas L J. The Earth Mover's Distance as a Metric for Image Retrieval. Int J Comput Vis 40, 99-121 (2000).
44. Hopfinger A J, Esposito E X, Llinas A, Glen R C, Goodman J M. Findings of the Challenge to Predict Aqueous Solubility. J Chem Inf Model 49, 1-5 (2009).
45. Llinas A, Oprisiu I, Avdeef A. Findings of the Second Challenge to Predict Aqueous Solubility. J Chem Inf Model 60, 4791-4803 (2020).
46. Llinas A, Avdeef A. Solubility Challenge Revisited after Ten Years, with Multilab Shake-Flask Data, Using Tight (SD Similar to 0.17 Log) and Loose (SD Similar to 0.62 Log) Test Sets. J Chem Inf Model 59, 3036-3040 (2019).
47. Llinas A, Glen R C, Goodman J M. Solubility Challenge: Can You Predict Solubilities of 32 Molecules Using a Database of 100 Reliable Measurements? J Chem Inf Model 48, 1289-1303 (2008).
48. Hinton G E, Salakhutdinov R R. Reducing the Dimensionality of Data with Neural Networks. Science 313, 504-507 (2006).
49. Zaheer M, Kottur S, Ravanbhakhsh S, Poczos B, Salakhutdinov R, Smola A J. Deep Sets. In: NIPS′17: Proceedings of the 31st International Conference on Neural Information Processing Systems) (2017).
50. Lee J, Bahri Y, Novak R, Schoenholz S S, Pennington J, Sohl-Dickstein J. Deep Neural Networks as Gaussian Processes. In: International Conference on Learning Representations) (2018).
51. Williams C K I, Rasmussen C E. Gaussian Processes for Regression. In: NIPS'95: Proceedings of the 9th Annual Conference on Neural Information Processing Systems) (1995).
52. Delaney J S. Esol: Estimating Aqueous Solubility Directly from Molecular Structure. J Chem Inf Comput Sci 44, 1000-1005 (2004).
53. Wu Z, Ramsundar B, Feinberg E N, Gomes J, Geniesse C, Pappu A S, Leswing K, Pande V. Moleculenet: A Benchmark for Molecular Machine Learning. Chem Sci 9, 513-530 (2018).
54. Fang X, Liu L, Lei J, He D, Zhang S, Zhou J, Wang F, Wu H, Wang H. Geometry-Enhanced Molecular Representation Learning for Property Prediction. Nature Machine Intelligence 4, 127-134 (2022).
55. Abraham M H, Le J. The Correlation and Prediction of the Solubility of Compounds in Water Using an Amended Solvation Energy Relationship. J Pharm Sci 88, 868-880 (1999).
56. van Tilborg D, Alenicheva A, Grisoni F. Exposing the Limitations of Molecular Machine Learning with Activity Cliffs. J Chem Inf Model 62, 5938-5951 (2022).
57. Barlow T W. Self-Organizing Maps and Molecular Similarity. J Mol Graph 13, 24-27 (1995).
58. Kulak T, Fillion A, Blayo F. A Unified View on Self-Organizing Maps (SOMEs) and Stochastic Neighbor Embedding (SNE). In: 31st International Conference on Artificial Neural Networks (eds Pimenidis E, Angelov P, Jayne C, Papaleonidas A, Aydin M). Springer (2022).
59. Wagener M, Sadowski J, Gasteiger J. Autocorrelation of Molecular Surface Properties for Modeling Corticosteroid Binding Globulin and Cytosolic Ah Receptor Activity by Neural Networks. J Am Chem Soc 117, 7769-7775 (1995).
60. Klamt A. Conductor-Like Screening Model for Real Solvents: A New Approach to the Quantitative Calculation of Solvation Phenomena. J Phys Chem 99, 2224-2235 (1995).
61. Gainza P, Sverrisson F, Monti F, Rodola E, Boscaini D, Bronstein M M, Correia B E. Deciphering Interaction Fingerprints from Protein Molecular Surfaces Using Geometric Deep Learning. Nat Methods 17, 184-192 (2020).
62. Sverrisson F, Feydy J, Correia B E, Bronstein M M. Fast End-to-End Learning on Protein Surfaces. In: IEEE/CVF Conference on Computer Vision and Pattern Recognition (CVPR)). IEEE (2021).
63. Jayatilaka D, Grimwood D J. Tonto: A Fortran Based Object-Oriented System for Quantum Chemistry and Crystallography. In: Computational Science-ICCS 2003, Pt Iv, Proceedings (eds Sloot P M A, Abramson D, Bogdanov A V, Dongarra J J, Zomaya A Y, Gorbachev Y E) (2003).
64. Cignoni P, Callieri M, Corsini M, Dellepiane M, Ganovelli F, Ranzuglia G. Meshlab: An Open-Source Mesh Processing Tool. In: Sixth Eurographics Italian Chapter Conference) (2008).
65. Xiao C, Hong S, Huang W D. Optimizing Graph Layout by T-SNE Perplexity Estimation. Int J Data Sci Anal.
66. Walt Svd, Schönberger J L, Nunez-Iglesias J, Boulogne F, Warner J D, Yager N, Gouillart E, Yu T. Scikit-Image: Image Processing in Python. PeerJ 2, e453 (2014).
67. Vaswani A, Shazeer N, Parmar N, Uszkoreit J, Jones L, Gomez A N, Kaiser L, Polosukhin I. Attention Is All You Need. In: NIPS'17: Proceedings of the 31st International Conference on Neural Information Processing Systems) (2017).

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A non-transitory computer-readable medium (CRM) comprising instructions that, when executed by at least one processor, cause the at least one processor to:
(a) receive a dataset of electronic quantities across a manifold topology of a molecule of interest;
(b) perform manifold regression on the electronic quantities to encode quantum information of the molecule in a covariant matrix (kernel), wherein the covariant matrix captures mutual relationships among the electronic quantities and the manifold topology; and
(c) export the covariance matrix as a symmetric semi-positive definite matrix for use as an input of molecular representation to a deep learning model configured to utilize the matrix for predicting molecular properties.

2. The CRM of claim 1, wherein the dataset of electronic quantities includes ESP and/or Fukui function quantities.

3. The CRM of claim 2, wherein the dataset of electronic quantities is transformed into a kernel matrix using Sparse Gaussian Process (SGP) with Spectral Mixture (SM) as the kernel function.

4. The CRM of claim 3, wherein the SGP utilizes a set of inducing points chosen as the closest vertices to the respective atoms of a molecule, resulting in the kernel matrix being n\hat{ }2, wherein n is the number of atoms in the molecule.

5. The CRM of claim 3, wherein the kernel function is a covariance function of the spectral solution of a manifold identified through an eigen decomposition of the Graph Laplacian of the molecular surface.

6. The CRM of claim 3 wherein the SGP utilizes a set of hyperparameters with the means and variances of the Gaussian functions set to 1.

7. The CRM of claim 4, wherein the dataset of electronic quantities is transformed into a combination of two or more kernel matrices, wherein the combination of resulting kernel matrices, derived from different electronic properties of the same molecule, is configured to predict the molecule's chemical properties.

8. The CRM of claim 1 wherein the deep learning mode is trained to receive a single positive definite (SPD) matrix as input to predict a molecule's chemical properties.

9. The CRM of claim 8, wherein the model is a SPDNet Attention model.

10. The CRM of claim 9, wherein the chemical properties are one or more of solubility, physicochemistry, drug target-binding efficiency/specificity, toxicity, ADME, drug developability, protein binding, or any molecular attribute that is determined by a molecule's electronic structure and quantities.

11. A method of generating unique descriptors of intermolecular interactions, the method comprising:
receiving a dataset of electronic quantities across a manifold topology of a molecule of interest;
performing manifold regression on the electronic quantities to encode quantum information of the molecule in a covariant matrix (kernel), wherein the covariant matrix captures mutual relationships among the electronic quantities and the manifold topology; and
exporting the covariance matrix as a symmetric semi-positive definite matrix for use as an input to a deep learning model configured to utilize the matrix for predicting a molecule's chemical properties.

12. The method of claim 11, wherein electronic quantities include ESP and/or Fukui function quantities of the molecule of interest, which is calculated by first principles.

13. The method of claim 12, wherein the electronic quantities are transformed into a kernel matrix using SGP with a kernel function such as SM.

14. The method of claim 13, wherein the SGP utilizes a set of inducing points chosen as the closest vertices to the respective atoms of a molecule, resulting in the kernel matrix being n\hat{ }2, wherein n is the number of atoms in the molecule.

15. The method of claim 13, wherein the kernel function is a covariance function of the spectral solution of a manifold identified through an eigen decomposition of the Graph Laplacian of the molecular surface.

16. The method of claim 13 wherein the SGP utilizes a set of hyperparameters with the means and variances of the Gaussian functions set to 1.

17. The method of claim 13, wherein the electronic quantities are transformed into a combination of two or more kernel matrices, wherein combination of resulting kernel matrices, derived from different electronic properties of the same molecule, is configured to predict the molecule's chemical properties.

18. The method of claim 11, wherein the deep learning model is trained to receive a single positive definite (SPD) matrix as input to predict the molecule's chemical properties.

19. The method of claim 18, wherein the deep learning model is a trained SPDNet Attention model.

20. The method of claim 18, wherein the chemical properties are one or more of solubility, developability, permeability, and absorption, distribution, metabolism, excretion, and/or toxicity (ADMET).
